(19) United States
(12) Patent Application Publication
(10) Pub. No.: US 2023/0381134 A1
(43) Pub. Date: Nov. 30, 2023

(54) COCRYSTALS OF CANNABINOIDS

(71) Applicant: PURISYS, LLC, ATHENS, GA (US)

(72) Inventors: Gnel Mkrtchyan, Morristown, NJ (US); JOSHUA K. HOERNER, WATKINSVILLE, GA (US); RICKY W. COUCH, DURHAM, NC (US); JOANNA A. BIS, CARY, NC (US); STEPHEN A.R. CARINO, RALEIGH, NC (US)

(21) Appl. No.: 17/758,151

(22) PCT Filed: Dec. 31, 2020
(86) PCT No.: PCT/US2020/067741

§ 371 Date: Jun. 29, 2022

Related U.S. Application Data
(60) Provisional application No. 62/957,023, filed on Jan. 3, 2020.

(51) Int. Cl. A61K 31/352; A61K 47/22

(52) U.S. Cl. CPC A61K 31/352; A61K 47/22

(57) ABSTRACT

Described herein are cocrystals comprising a coformer and a cannabinoid selected from the group consisting of cannabinol and tetrahydrocannabinol. In certain embodiments, the coformer is tetramethylpyrazine, L-proline, or D-proline. Further described are methods of preparing the cocrystals starting from a cannabinoid oil and pharmaceutical compositions comprising the cocrystals.

### CROSS REFERENCE TO RELATED APPLICATIONS

This application claims the benefit of priority to U.S. Provisional Patent Application No. 62/957,023, filed on Jan. 3, 2020, the contents of which are incorporated by reference herein in their entirety for all purposes.

### FIELD

The subject matter described herein relates to novel solid forms comprising a cannabinoid and methods for the preparation of the solid forms starting from a cannabinoid oil.

### BACKGROUND

The characterization and selection of a solid compound for use in a pharmaceutical composition is a complex process, as even the slightest modifications in the solid's form may affect the compound's physical and chemical properties. These properties may offer potential drawbacks or advantages affecting the composition's pharmaceutical characteristics, such as processing, formulation, stability, bioavailability, storage, and handling. Crystalline solids and amorphous solids are used as solids in pharmaceutical compositions, with the product type and mode of administration often influencing the choice of solid material. Crystalline solids are characterized by structural periodicity, while amorphous solids lack long-range structural order. The selection of a particular solid may depend on the specific application; amorphous solids are sometimes selected on the basis of, e.g., an enhanced dissolution profile, while crystalline solids may be desirable for properties such as, e.g., physical or chemical stability (see, e.g., S. R. Vippagunta et al., Adv. Drug. Deliv. Rev., (2001) 48:3-26; L. Yu, Adv. Drug. Deliv. Rev., (2001) 48:27-42).

Regardless of whether the solid material is crystalline or amorphous, a pharmaceutical composition comprising a solid form may contain single-component and multiple-component solids. Single-component solids consist essentially of the pharmaceutical compound or active pharmaceutical ingredient without any other compounds. Variability among single-component crystalline materials could potentially develop as a result of polymorphism, a phenomenon characterized by the existence of several three-dimensional crystalline arrangements for a single pharmaceutical compound (see, e.g., S. R. Byrn et al., Solid State Chemistry of Drugs, (1999) SSCI, West Lafayette). Several factors to consider in the design of a crystalline form of a therapeutic agent is that it retains its polymorphic and chemical stability, solubility, and other physiochemical properties over time and among various manufactured batches of the agent. If the physiochemical properties vary with time and among batches, the administration of a therapeutically effective dose becomes problematic and may lead to toxic side effects or to ineffective therapy, particularly if a given polymorph decomposes prior to use, to a less active, or toxic compound. The importance of identifying polymorphs in pharmaceutical compositions was highlighted in the case of Ritonavir™, an HIV protease inhibitor formulated as soft gelatin capsules. Roughly two years after the product launched, a new, less soluble polymorph had been discovered in the formulation, which required the withdrawal of the original capsules from the market and reformulation of the product (see S. R. Chemburkar et al., Org. Process Res. Dev., (2000) 4:413-417).

The possibility of multi-component solids may provide additional diversity among the potential solid forms of a pharmaceutical compound. Crystalline solids comprising two or more ionic species are typically referred to as salts (see, e.g., Handbook of Pharmaceutical Salts: Properties, Selection and Use, P. H. Stahl and C. G. Wermuth, Eds., (2002), Wiley, Weinheim). Other types of multiple-component solids that could influence the properties of a pharmaceutical compound or salt thereof include, e.g., hydrates, solvates, cocrystals and clathrates, among others (see, e.g., S. R. Byrn et al., Solid State Chemistry of Drugs, (1999) SSCI, West Lafayette). Furthermore, multiple-component crystal forms may undergo polymorphism, wherein a given multiple-component composition may exist in more than one three-dimensional crystalline arrangement.

Cocrystals are crystalline molecular complexes comprising two or more non-volatile compounds bound together by non-ionic interactions in a crystal lattice. Pharmaceutical cocrystals are cocrystals of a therapeutic compound, e.g., an active pharmaceutical ingredient (API), and one or more non-volatile compound(s) (referred to herein as a coformer or cocrystal former). A coformer in a pharmaceutical cocrystal is typically a non-toxic pharmaceutically acceptable molecule. Non-limiting examples of coformers include food additives, preservatives, pharmaceutical excipients, or other APIs. A cocrystal comprising an API and one or more coformers is a distinct chemical composition. As such, the cocrystal generally exemplifies distinct crystallographic and spectroscopic properties when compared to its individual API and coformer components. In the past several years, pharmaceutical cocrystals have emerged as a possible alternative approach to enhance physicochemical properties of drug products. For example, in comparison to the API, a cocrystal may offer attractive dissolution and/or solubility properties, storage stability, compressibility and density (useful in formulation and product manufacturing), permeability, and hydrophilic or lipophilic character.

It is worthwhile to point out that it is not possible to predict a priori if crystalline forms of a compound even exist, let alone how to successfully prepare them (see, e.g., Braga and Grepioni, 2005, “Making crystals from crystals: a green route to crystal engineering and polymorphism,” Chem. Commun.: 3635-3645 (with respect to crystal engineering, if instructions are not very precise and/or if other external factors affect the process, the result can be unpredictable); Jones et al., 2006, Pharmaceutical Cocrystals: An Emerging Approach to Physical Property Enhancement,” MRS Bulletin 31:875-879 (At present it is not generally possible to computationally predict the number of observable polymorphs of even the simplest molecules); Price, 2004, “The computational prediction of pharmaceutical crystal structures and polymorphism,” Advanced Drug Delivery Reviews 56:301-319 (“Price”); and Bernstein, 2004, “Crystal Structure Prediction and Polymorphism,” ACA Transactions 39:14-23 (a great deal still needs to be learned and done before one can state with any degree of confidence the ability to predict a crystal structure, much less polymorphic forms)).

The existence of many types of solid forms allows for diversity in evaluating the physical properties of a pharmaceutical compound. As such, the discovery and choice of solid compounds are of great importance in the development of an effective, stable, and marketable pharmaceutical product.

The cannabis plant has many naturally occurring substances. Many substances are available primarily as oils. What is needed, therefore, is solid, crystalline forms that can have advantageous properties, including those described herein.

### BRIEF SUMMARY

In certain aspects, the subject matter described herein is directed to a solid form comprising a coformer and a cannabinoid selected from the group consisting of cannabinol and tetrahydrocannabinol.

In certain aspects, the subject matter described herein is directed to a cocrystal comprising cannabinol and tetramethylpyrazine.

In certain aspects, the subject matter described herein is directed to a cocrystal comprising cannabinol and L-proline.

In certain aspects, the subject matter described herein is directed to a cocrystal comprising cannabinol and D-proline.

In certain aspects, the subject matter described herein is directed to a method of preparing a solid form comprising a coformer and a cannabinoid, starting from a cannabinoid oil.

These and other aspects are described fully herein.

### BRIEF DESCRIPTION OF THE FIGURES

FIG. 1 depicts a Fourier Transform-Raman spectrum of the CBN starting material.

FIG. 2 depicts a ¹H NMR spectrum of the CBN starting material.

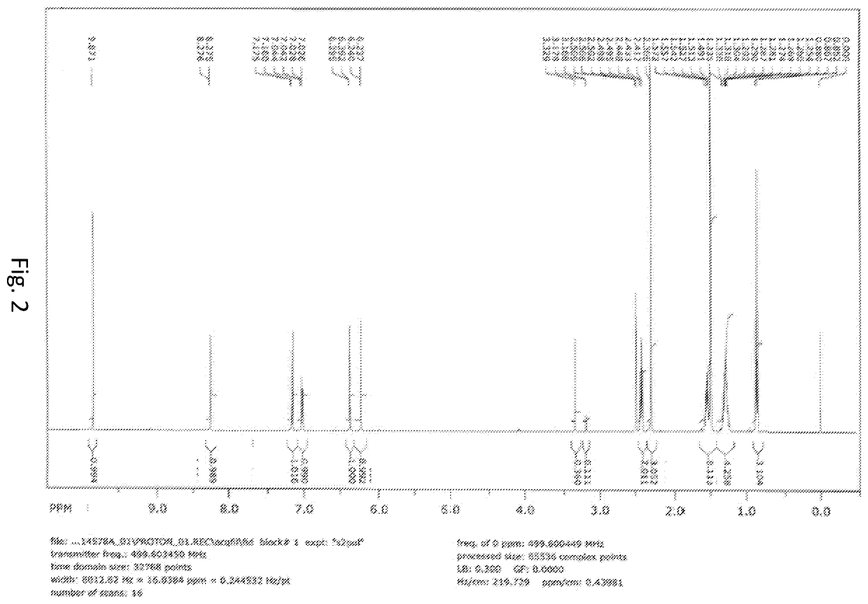

FIG. 3 depicts a thermogravimetric analysis interfaced with infrared spectrophotometer analysis of the CBN starting material.

FIG. 4 depicts a PLM (polarized light microscope) image of the CBN starting material.

FIG. 5 depicts a Fourier Transform-Raman spectrum of Hemi-TMP Cocrystal Group A (Non-Solvated).

FIG. 6 depicts a powder X-ray diffraction pattern of Hemi-TMP Cocrystal Group A (Non-Solvated).

FIG. 7 depicts a differential scanning calorimetry/thermogravimetric plot of Hemi-TMP Cocrystal Group A (Non-Solvated).

FIG. 8 depicts a ¹H NMR spectrum of Hemi-TMP Cocrystal Group A (Non-Solvated).

FIG. 9 depicts a Fourier Transform-Raman spectrum of Mono-TMP Cocrystal Group B (Non-Solvated).

FIG. 10 depicts a powder X-ray diffraction pattern of Mono-TMP Cocrystal Group B (Non-Solvated).

FIG. 11 depicts a differential scanning calorimetry/thermogravimetric plot of Mono-TMP Cocrystal Group B (Non-Solvated).

FIG. 12 depicts a PLM (polarized light microscope) image of Mono-TMP Cocrystal Group B (Non-Solvated).

FIG. 13 depicts a ¹H NMR spectrum of Mono-TMP Cocrystal Group B (Non-Solvated).

FIG. 14 depicts a Fourier Transform-Raman spectrum of Mono-L-Proline Cocrystal Group C (Non-Solvated).

FIG. 15 depicts a powder X-ray diffraction pattern of Mono-L-Proline Cocrystal Group C (Non-Solvated).

FIG. 16 depicts a differential scanning calorimetry/thermogravimetric plot of Mono-L-Proline Cocrystal Group C (Non-Solvated).

FIG. 17 depicts a PLM (polarized light microscope) image of Mono-L-Proline Cocrystal Group C (Non-Solvated).

FIG. 18 depicts a ¹H NMR spectrum of Mono-L-Proline Cocrystal Group C (Non-Solvated).

FIG. 19 depicts a Fourier Transform-Raman spectrum of Mono-L-Proline Cocrystal Group B (Isooctane Solvate).

FIG. 20 depicts a powder X-ray diffraction pattern of Mono-L-Proline Cocrystal Group B (Isooctane Solvate).

FIG. 21 depicts a differential scanning calorimetry/thermogravimetric plot of Mono-L-Proline Cocrystal Group B (Isooctane Solvate).

FIG. 22 depicts a PLM (polarized light microscope) image of Mono-L-Proline Cocrystal Group B (Isooctane Solvate).

FIG. 23 depicts a ¹H NMR spectrum of Mono-L-Proline Cocrystal Group B (Isooctane Solvate).

FIG. 24 depicts a Fourier Transform-Raman spectrum of L-Proline Cocrystal Group B (Petroleum Ether Solvate).

FIG. 25 depicts a powder X-ray diffraction pattern of L-Proline Cocrystal Group B (Petroleum Ether Solvate).

FIG. 26 depicts a differential scanning calorimetry/thermogravimetric plot of L-Proline Cocrystal Group B (Petroleum Ether Solvate).

FIG. 27 depicts a PLM (polarized light microscope) image of L-Proline Cocrystal Group B (Petroleum Ether Solvate).

FIG. 28 depicts a Fourier Transform-Raman spectrum of Mono-D-Proline Cocrystal Group C (Non-Solvated).

FIG. 29 depicts a powder X-ray diffraction pattern of Mono-D-Proline Cocrystal Group C (Non-Solvated).

FIG. 30 depicts a differential scanning calorimetry/thermogravimetric plot of Mono-D-Proline Cocrystal Group C (Non-Solvated).

FIG. 31 depicts a PLM (polarized light microscope) image of Mono-D-Proline Cocrystal Group C (Non-Solvated).

FIG. 32 depicts a ¹H NMR spectrum of Mono-D-Proline Cocrystal Group C (Non-Solvated).

FIG. 33 depicts a Fourier Transform-Raman spectrum of Mono-D-Proline Cocrystal Group B (Isooctane Solvate).

FIG. 34 depicts a powder X-ray diffraction pattern of Mono-D-Proline Cocrystal Group B (Isooctane Solvate).

FIG. 35 depicts a differential scanning calorimetry/thermogravimetric plot of Mono-D-Proline Cocrystal Group B (Isooctane Solvate).

FIG. 36 depicts a PLM (polarized light microscope) image of Mono-D-Proline Cocrystal Group B (Isooctane Solvate).

FIG. 37 depicts a ¹H NMR spectrum of Mono-D-Proline Cocrystal Group B (Isooctane Solvate).

FIG. 38 depicts a Fourier Transform-Raman spectrum of D-Proline Cocrystal Group B (Petroleum Ether Solvate).

FIG. 39 depicts a powder X-ray diffraction pattern of D-Proline Cocrystal Group B (Petroleum Ether Solvate).

FIG. 40 depicts a differential scanning calorimetry/thermogravimetric plot of D-Proline Cocrystal Group B (Petroleum Ether Solvate).

FIG. 41 depicts a PLM (polarized light microscope) image of D-Proline Cocrystal Group B (Petroleum Ether Solvate).

FIG. 42 shows an overlay of powder X-ray diffraction patterns for Groups A and B of the Tetramethylpyrazine cocrystal.

FIG. 43 shows an overlay of powder X-ray diffraction patterns for Groups A, B, and C of the L-Proline cocrystal.

FIG. 44 shows an overlay of powder X-ray diffraction patterns for L-Proline Cocrystal Group B After 18 Hours and 14 Days at Ambient and the CCF.

FIG. 45 shows an overlay of powder X-ray diffraction patterns for Groups A, B, and C of the D-Proline cocrystal.

FIG. 46 shows an overlay of powder X-ray diffraction patterns for potential nicotinamide cocrystals.

FIG. 47 shows DSC Cycling data for the starting CBN starting material.

FIG. 48 shows an overlay of powder X-ray diffraction patterns for the TMP cocrystals (Hemi-TMP Cocrystal Group A and Mono-TMP Cocrystal Group B) in comparison with the tetramethylpyrazine coformer.

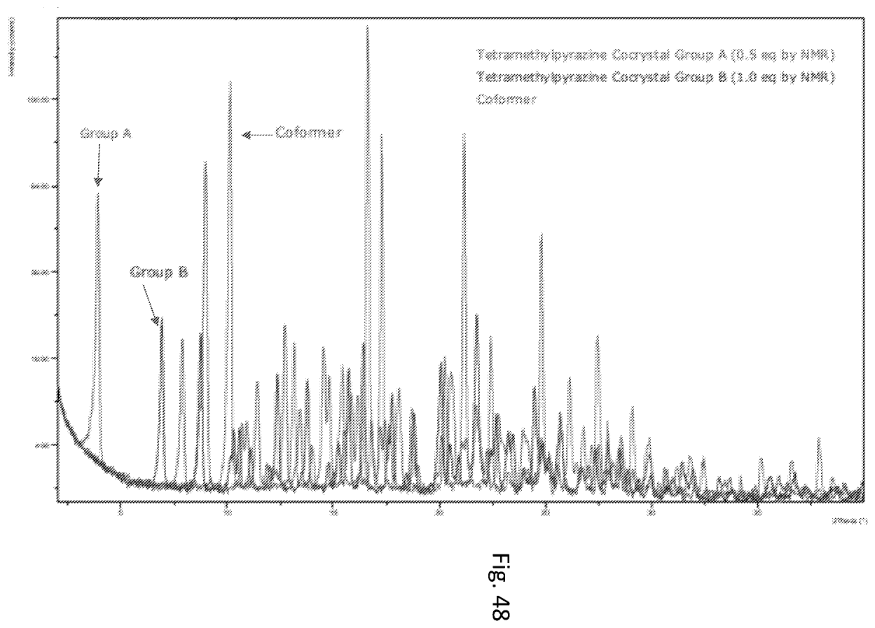

FIG. 49 shows an overlay of powder X-ray diffraction patterns for the L-Proline cocrystals (Group B and Group C) in comparison with the L-Proline coformer.

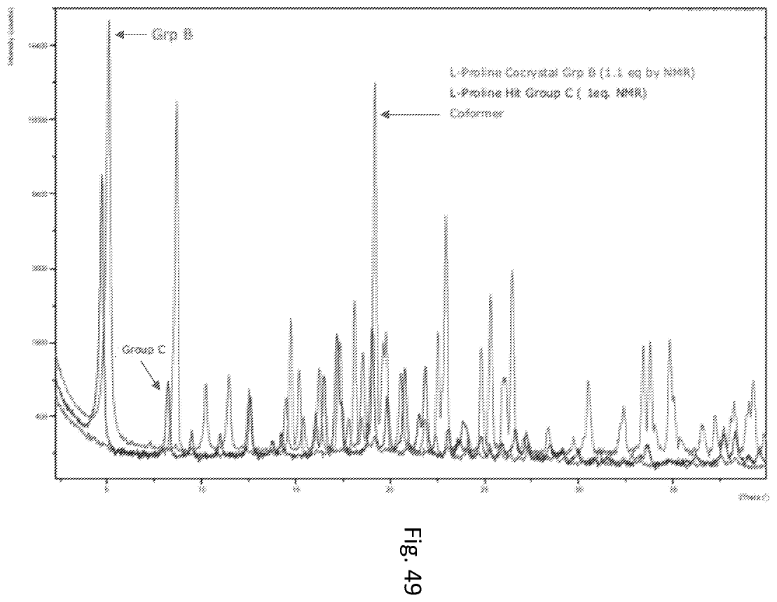

FIG. 50 shows an overlay of powder X-ray diffraction patterns for the D-Proline cocrystals (Group B and Group C) in comparison with the D-Proline coformer.

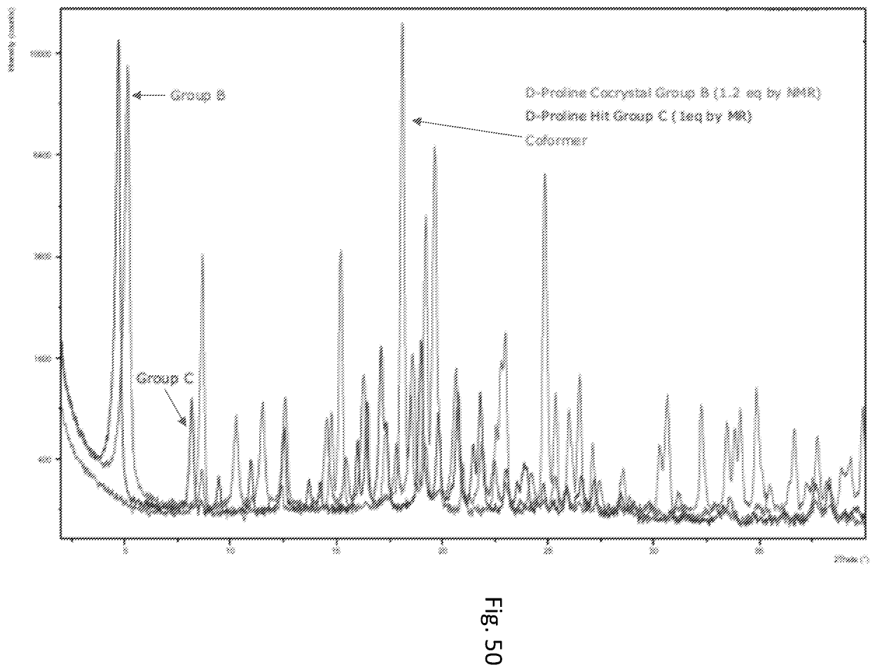

FIG. 51 shows an overlay of powder X-ray diffraction patterns for the Hemi-TMP Cocrystal Group A before and after 3 days, along with the TMP coformer profile for comparison.

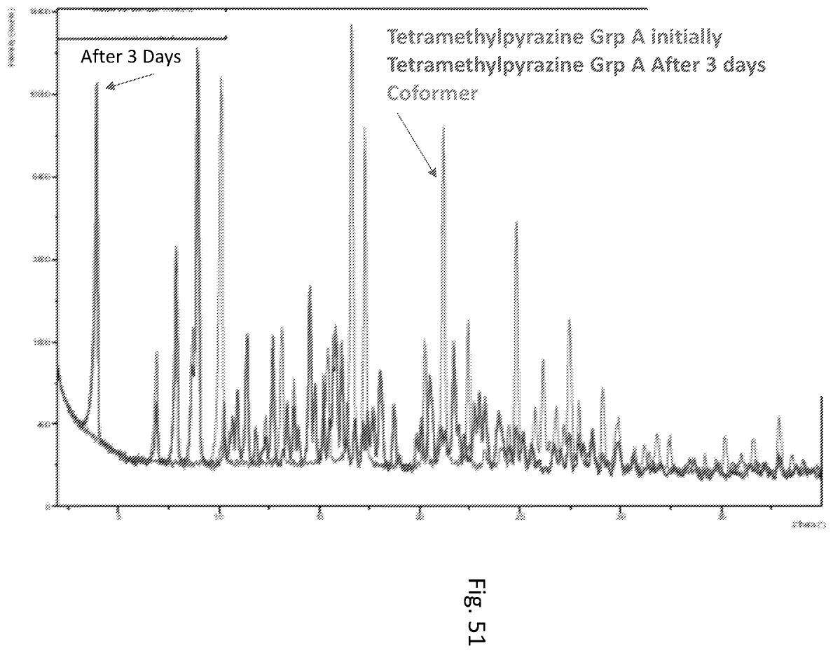

### DETAILED DESCRIPTION

Disclosed herein are novel solid forms comprising a cannabinoid and methods for the preparation of the solid forms starting from a cannabinoid oil. Cannabinoids, such as cannabinol (CBN) or delta-9-tetrahydrocannabinol (THC), may be isolated by extraction or cold pressing from cannabis plants or prepared synthetically. Other examples of cannabinoids include cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol monomethylether (CBDM), cannabidiol-C.sub.4 (CBD-C.sub.4), cannabidivarinic acid (CBDVA), cannabidivarin (CBDV), cannabidiorcol (CBD-C.sub.1), delta-9-tetrahydrocannabinolic acid A (THCA-A), delta-9-tetrahydrocannabinolic acid B (THCA-B), delta-9-tetrahydrocannabinolic acid-C.sub.4 (THCA-C.sub.4), delta-9-tetrahydrocannabinol-C.sub.4 (THC-C.sub.4), delta-9-tetrahydrocannabivarinic acid (THCVA), delta-9-tetrahydrocannabivarin (THCV), delta-9-tetrahydrocannabiorcolic acid (THCA-C.sub.1), delta-9-tetrahydrocannabiorcol (THC-C.sub.1), delta-7-cis-iso-tetrahydrocannabivarin, delta-8-tetrahydrocannabinolic acid (.DELTA.sup.8-THCA), delta-8-tetrahydrocannabinol (.DELTA.sup.8-THC), cannabicyclolic acid (CBLA), cannabicyclol (CBL), cannabicyclovarin (CBLV), cannnabielsoic acid A (CBEA-A), cannabielsoic acid B (CBEA-B), cannabielsoin (CBE), cannabinolic acid (CBNA), cannabinol (CBN), cannabinol methylether (CBNM), cannabinol-C.sub.4 (CBN-C.sub.4), cannabivarin (CBV), cannabinol-C.sub.2 (CBN-C.sub.2), cannabiorcol (CBN-C.sub.1), cannabinodiol (CBND), cannabinodivarin (CBVD), cannabitriol (CBT), 10-ethoxy-9-hydroxy-delta-6a-tetrahydrocannabinol, 8,9-dihydroxy-delta-6a-tetrahydrocannabinol, cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2,6-metha-no-2H-1-benzoxocin-5-methanol (OH-iso-HHCV), cannabiripsol (CBR) and trihydroxy-delta-9-tetrahydrocannabinol (triOH-THC). Cannabinoids have been investigated for possible treatment of seizures, nausea, vomiting, lack of appetite, pain, arthritis, inflammation, and other conditions.

The IUPAC nomenclature for cannabinol is 6,6,9-Trimethyl-3-pentyl-benzo[c]chromen-1-ol. Its chemical structure is presented below:

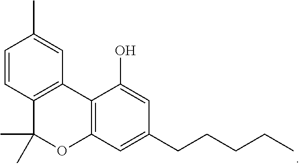 [SMILES: CCCCCc1cc(O)c2c(c1)OC(C)(C)c1ccc(C)cc1-2]

Cannabinol is a mildly psychoactive cannabinoid found only in trace amounts in cannabis. In comparison, the most notable cannabinoid found in cannabis is tetrahydrocannabinol, the primary psychoactive compound in cannabis. The IUPAC nomenclature for tetrahydrocannabinol is (−)-(6aR,10aR)-6,6,9-trimethyl-3-pentyl-6a,7,8,10a-tetrahydro-6H-benzo[c]chromen-1-ol. Its chemical structure is presented below:

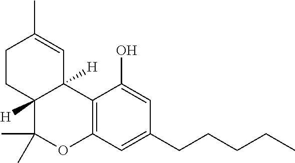 [SMILES: CCCCCc1cc(O)c2c(c1)OC(C)(C)[C@@H]1CCC(C)=C[C@@H]21]

It has been reported that stored, degraded or oxidized cannabis products, such as low-quality baled cannabis and traditionally produced hashish, are higher in CBN. By way of example, when cannabis is exposed to air or ultraviolet light (for example, in sunlight) for a prolonged period, tetrahydrocannabinolic acid (THCA) can convert to cannabinolic acid (CBNA). CBN is then formed by the decarboxylation of CBNA. CBN is also formed as a metabolite of THC.

CBN has potential immunosuppressive and anti-inflammatory activities. Cannabinol preferentially binds to the cannabinoid G-protein coupled receptor CB2, which is mainly expressed on a variety of immune cells, such as T-cells, B-cells, macrophages and dendritic cells. Stimulation of CB2 receptors by cannabinol may both trigger apoptosis in these cells and inhibit the production of a variety of cytokines. Cannabinol exerts minimal affinity for CB1 and has a weak effect on the central nervous system. Relative to THC, CBN has lower affinities for each receptor, about 7- to 8-fold lower for CB1 receptor and about 3-fold lower for CB2 receptor.

The potential therapeutic value of both CBN and THC in the treatment of diseases has stimulated research and development in formulating the cannabinoids for use in pharmaceutical compositions. Cannabinoids are generally highly lipophilic molecules (log P 6-7) with very low aqueous solubility (2-10 μg/mL), that are susceptible to degradation, especially in solution, via the action of light and temperature as well as via auto-oxidation (Grotenhermen F. Clin. Pharmacokinet. 2003; 42:327-360; Pacifici R. et al. Clin. Chem. Lab. Med. 2018; 56:e94-e96; Fairbairn J. W. et al. J. Pharm. Pharmacol. 1976; 28:1-7). Formulation can thus play a crucial role in increasing the solubility and physicochemical stability of the drugs. In particular, CBN and THC are typically found as oils, whether derived from plants or synthetically prepared. Likewise, other cannabinoids can be useful but are limited because they are oils. Solid forms of CBN, THC, and other cannabinoids, such as crystalline compositions, could be more advantageous in one or more respects compared to other compositions of matter comprising the cannabinoids, for example, in terms of chemical and physical stability, storage, processing, compatibility, and hygroscopicity. It is also possible that crystalline compositions could offer easier, quicker, and more extensive dissolution into solvents and more rapid bioavailability when compared to other forms of the cannabinoids.

Disclosed herein are novel solid cannabinoid compositions and methods for their preparation. The present methods allow for the preparation of solid cannabinoid forms from the oils of lipophilic cannabinoids. The cannabinoid oil starting materials can be obtained as extracts from the cannabis plant or prepared synthetically in the lab. In certain embodiments, the solid cannabinoid forms are cocrystals, comprising a cannabinoid and a coformer.

The crystallization of lipophilic materials is a complex process. Problems, such as polymorphic transitions, oil migration, slow crystallization, and the formation of crystalline aggregates are often encountered. However, it has been found that oily cannabinoids, such as cannabinol, can crystallize into cocrystal compositions with a coformer. As described herein, combining the oily cannabinoid with an appropriate coformer and solvent with stirring and temperature cycling can produce cocrystalline cannabinoid compositions in high yields. Without wishing to be bound by theory, it is understood that temperature-cycling facilitates dissolution and recrystallization in the cannabinoid crystal suspension. During crystallization of the cocrystal, the fine crystals of one species are expected to dissolve out completely during the heating period, and the remaining crystals or dominant species continuously grow or nucleate during the cooling period. This, in part, provides cannabinoid cocrystals with exceptional crystallinity, stability, and purity.

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. In the event that one or more of the incorporated literature, patents, and similar materials differs from or contradicts this application, including but not limited to defined terms, term usage, described techniques, or the like, this application controls. Unless otherwise defined, all technical and scientific terms used herein have the same meaning as commonly understood by one of ordinary skill in this field. All publications, patent applications, patents, and other references mentioned herein are incorporated by reference in their entirety.

### I. Definitions

As used herein, the “CBN” refers to cannabinol, “CBD” refers to cannabidiol, and “THC” refers to tetrahydrocannabinol.

As used herein, “CCF” refers to cocrystal former or coformer.

As used herein, “FT-Raman” refers to Fourier-transform Raman spectroscopy.

As used herein, “PXRD” refers to powder X-ray diffraction.

As used herein, “DSC” refers to differential scanning calorimetry.

As used herein, “TGA-IR” refers to thermogravimetric analysis interfaced with infrared spectrometry.

As used herein, “TMP” refers to tetramethylpyrazine.

As used herein, the term “non-solid” refers to a liquid or semi-liquid (viscous) preparations, such as an oil. An oil can be an extract or a synthetic preparation.

As used herein, “stable” is intended to mean that the cocrystal composition maintains its crystallinity, as monitored by PXRD, and is not readily decomposing to its individual coformer or cannabinoid components.

As used herein, the term “contacting” refers to allowing two or more reagents to contact each other. The contact may or may not be facilitated by mixing, agitating, stirring, and the like.

As used herein, “API” refers to Active Pharmaceutical Ingredient.

As used herein, “substantially free of solvent” refers to a composition that is essentially non-solvated.

As used herein, and unless otherwise indicated, a chemical compound, solid form, or composition that is “substantially free” of another chemical compound, solid form, or composition means that the compound, solid form, or composition contains, in certain embodiments, less than about 50%, 45%, 40%, 35%, 30%, 25%, 20%, 15%, 10%, 9%, 8%, 7%, 6%, 5%, 4%, 3%, 2%, 1%, 0.5%, 0.4%, 0.3%, 0.2% 0.1%, 0.05%, or 0.01% by weight of the other compound, solid form, or composition. As used herein, “essentially free” refers to levels that are below trace. In certain embodiments, essentially free refers to amounts not detectable by standard techniques.

As used herein, the term “crystalline” and related terms used, when used to describe a substance, component, product, or form, mean that the substance, component, product, or form is substantially crystalline, for example, as determined by X-ray diffraction. (see, e.g., Remington's Pharmaceutical Sciences, 20^{th} ed., Lippincott Williams & Wilkins, Philadelphia Pa., 173 (2000); The United States Pharmacopeia, 37^{th} ed., 503-509 (2014)).

As used herein, and unless otherwise specified, a solid form that is “substantially chemically pure” is substantially free from other chemical compounds (i.e., chemical impurities). In certain embodiments, a solid form that is substantially chemically pure contains less than about 50%, 45%, 40%, 35%, 30%, 25%, 20%, 15%, 10%, 9%, 8%, 7%, 6%, 5%, 4%, 3%, 2%, 1%, 0.5%, 0.4%, 0.3%, 0.2%, 0.1%, 0.05%, or 0.01% of one or more other chemical compounds on a weight basis. The detection of other chemical compounds can be accomplished by any method apparent to a person of ordinary skill in the art, including, but not limited to, methods of chemical analysis, such as, e.g., mass spectrometry analysis, spectroscopic analysis, thermal analysis, elemental combustion analysis and/or chromatographic analysis.

As used herein, and unless otherwise specified, a solid form that is “substantially physically pure” is substantially free from other solid forms. In certain embodiments, a crystal form that is substantially physically pure contains less than about 50%, 45%, 40%, 35%, 30%, 25%, 20%, 15%, 10%, 9%, 8%, 7%, 6%, 5%, 4%, 3%, 2%, 1%, 0.5%, 0.4%, 0.3%, 0.2%, 0.1%, 0.05%, or 0.01% of one or more other solid forms on a weight basis. The detection of other solid forms can be accomplished by any method apparent to a person of ordinary skill in the art, including, but not limited to, diffraction analysis, thermal analysis, elemental combustion analysis and/or spectroscopic analysis.

Unless otherwise specified, the term “composition” as used herein is intended to encompass a product comprising the specified ingredient(s) (and in the specified amount(s), if indicated), as well as any product which results, directly or indirectly, from combination of the specified ingredient(s) in the specified amount(s). Additionally, the term “composition” refers to a mixture of compounds.

Unless otherwise specified, to the extent that there is a discrepancy between a depicted chemical structure of a compound provided herein and a chemical name of a compound provided herein, the chemical structure shall control.

As used herein, and unless otherwise specified, the terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more prophylactic or therapeutic agents to a subject with such a disease or disorder. In some embodiments, the terms refer to the administration of a compound provided herein, with or without other additional active agent, after the onset of symptoms of a particular disease.

A “patient” or “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the patient, individual, or subject is a human.

As used herein, the term “therapeutic amount” refers to an amount of a therapeutic agent, compound, formulation, material, or composition, as described herein effective to achieve a particular biological result. Such results may include, but are not limited to, the inhibition of a disease as determined by any means suitable in the art.

As used herein, the term “pharmaceutically acceptable excipient” refers to an ingredient in a pharmaceutical formulation, other than an active ingredient, which is nontoxic to a subject. A pharmaceutically acceptable excipient includes, but is not limited to, a buffer, carrier, stabilizer, or preservative. By “pharmaceutically acceptable,” it is meant a diluent, excipient, or carrier in a formulation must be compatible with the other ingredient(s) of the formulation and not deleterious to the recipient thereof.

As used herein, and unless otherwise specified, the terms “about” and “approximately,” when used in connection with a numeric value or range of values which is provided to characterize a particular solid form, e.g., a specific temperature or temperature range, such as, for example, that describes a melting, dehydration, desolvation, or glass transition temperature; a mass change, such as, for example, a mass change as a function of temperature or humidity; a solvent or water content, in terms of, for example, mass or a percentage; or a peak position, such as, for example, in analysis by, for example, IR or Raman spectroscopy or PXRD; indicate that the value or range of values may deviate to an extent deemed reasonable to one of ordinary skill in the art while still describing the solid form. Techniques for characterizing crystal forms and amorphous forms include, but are not limited to, thermal gravimetric analysis (TGA), differential scanning calorimetry (DSC), powder X-ray powder diffraction (PXRD), single-crystal X-ray diffraction, vibrational spectroscopy, e.g., infrared (IR) and Raman spectroscopy, solid-state and solution nuclear magnetic resonance (NMR) spectroscopy, optical microscopy, hot stage optical microscopy, scanning electron microscopy (SEM), electron crystallography and quantitative analysis, particle size analysis (PSA), surface area analysis, solubility studies, and dissolution studies. In certain embodiments, the terms “about” and “approximately,” when used in this context, indicate that the numeric value or range of values may vary within 30%, 20%, 15%, 10%, 9%, 8%, 7%, 6%, 5%, 4%, 3%, 2%, 1.5%, 1%, 0.5%, or 0.25% of the recited value or range of values. In the context of molar ratios, “about” and “approximately” indicate that the numeric value or range of values may vary within 20%, 15%, 10%, 9%, 8%, 7%, 6%, 5%, 4%, 3%, 2%, 1.5%, 1%, 0.5%, or 0.25% of the recited value or range of values. It should be understood that the numerical values of the peaks of a powder X-ray powder diffraction pattern may vary from one machine to another, or from one sample to another, and so the values quoted are not to be construed as absolute, but with an allowable variability, such as ±0.20 degrees two theta (° 20), or more. For example, in some embodiments, the value of a PXRD peak position may vary by up to ±0.20 degrees 2θ or ±0.2 degrees 2θ while still describing the particular PXRD peak.

Additional definitions are provided below.

### II. Solid Form Compositions

The subject matter described herein is directed to a solid form comprising a coformer and a cannabinoid. The cannabinoid can be one or more of cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol (CBD), cannabidiol monomethylether (CBDM), cannabidiol-C.sub.4 (CBD-C.sub.4), cannabidivarinic acid (CBDVA), cannabidivarin (CBDV), cannabidiorcol (CBD-C.sub.1), delta-9-tetrahydrocannabinolic acid A (THCA-A), delta-9-tetrahydrocannabinolic acid B (THCA-B), delta-9-tetrahydrocannabinol (THC), delta-9-tetrahydrocannabinolic acid-C.sub.4 (THCA-C.sub.4), delta-9-tetrahydrocannabinol-C.sub.4 (THC-C.sub.4), delta-9-tetrahydrocannabivarinic acid (THCVA), delta-9-tetrahydrocannabivarin (THCV), delta-9-tetrahydrocannabiorcolic acid (THCA-C.sub.1), delta-9-tetrahydrocannabiorcol (THC-C.sub.1), delta-7-cis-iso-tetrahydrocannabivarin, delta-8-tetrahydrocannabinolic acid (.DELTA.sup.8-THCA), delta-8-tetrahydrocannabinol (.DELTA.sup.8-THC), cannabicyclolic acid (CBLA), cannabicyclol (CBL), cannabicyclovarin (CBLV), cannnabielsoic acid A (CBEA-A), cannabielsoic acid B (CBEA-B), cannabielsoin (CBE), cannabinolic acid (CBNA), cannabinol (CBN), cannabinol methylether (CBNM), cannabinol-C.sub.4 (CBN-C.sub.4), cannabivarin (CBV), cannabinol-C.sub.2 (CBN-C.sub.2), cannabiorcol (CBN-C.sub.1), cannabinodiol (CBND), cannabinodivarin (CBVD), cannabitriol (CBT), 10-ethoxy-9-hydroxy-delta-6a-tetrahydrocannabinol, 8,9-dihydroxy-delta-6a-tetrahydrocannabinol, cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2,6-metha-no-2H-1-benzoxocin-5-methanol (OH-iso-HHCV), cannabiripsol (CBR) and trihydroxy-delta-9-tetrahydrocannabinol (triOH-THC). In certain embodiments, useful cannabinoids are those that are not solids at ambient conditions (about 25 degrees Celsius and 1 atm). Particularly useful are cannabinoids that are oils, such as CBN or THC.

By “coformer” or “cocrystal former” what is meant is the component of the cocrystal that is not the compound of the cocrystal. The coformer is present in order to form the cocrystal with the compound. Thus, the coformer is part of the crystal lattice. It is contemplated that one or more coformers may be employed in a cocrystal, according to any of the methods described herein.

The coformer may be non-ionized, such as, for example, benzoic acid, succinic acid, and caffeine, or zwitterionic, such as, for example, L-lysine, L-arginine, or L-proline, or may be a salt, such as, for example, sodium benzoate or sodium succinate. Coformers may include, but are not limited to, organic bases, organic salts, alcohols, aldehydes, amino acids, sugars, ionic inorganics, carboxylic acids, amines, flavoring agents, sweeteners, nutraceuticals, aliphatic esters, aliphatic ketones, organic acids, aromatic esters, alkaloids, and aromatic ketones. The coformer may be a carboxylic acid or an alkaloid. Typically, coformers will have the ability to form complementary non-covalent interactions with the compound or its salt, including APIs and salts thereof, for example the ability to form hydrogen bonds with the compound or its salt.

In certain embodiments, the coformer is selected from the group consisting of acetylsalicylic acid, D-glucose, nicotinic acid, aconitic acid, L-glutamic acid, oxalic acid, adipic acid, glutaric acid, L-proline, 4-aminosalicylic acid, glycine, propyl gallate, L-ascorbic acid, glycolic acid, L-pyroglutamic acid, benzoic acid, hippuric acid, saccharin, (+)-camphoric acid, 1-hydroxy-2-naphthoic acid, salicylic acid, capric acid, ketoglutaric acid, sebacic acid, cinnamic acid, L-lysine, sodium lauryl sulfate, citric acid, magnesium bromide, sorbic acid, cyclamic acid, maleic acid, succinic acid, ethyl maltol, L-malic acid, L-tartaric acid, ethyl paraben, malonic acid, urea, D-fructose, maltol, vanillic acid, fumaric acid, D,L-mandelic acid, vanillin, gallic acid, methyl paraben, zinc chloride, gentisic acid, potassium, calcium, carnitine, aspartame, D-proline, betaine, L-Arginine, tetramethylpyrazine, 1H-Imidazole, and nicotinamide.

In certain embodiments, the coformer is selected from the group consisting of potassium, calcium, carnitine, aspartame, L-proline, D-proline, L-Arginine, L-Lysine, betaine, tetramethylpyrazine, 1H-Imidazole, nicotinic acid, saccharin, urea, and nicotinamide.

In certain embodiments of the solid form, the cannabinoid is cannabinol and the coformer is tetramethylpyrazine.

In certain embodiments of the solid form, the molar ratio of cannabinol to tetramethylpyrazine is about 1 to 0.5.

In certain embodiments, the solid form comprising cannabinol and tetramethylpyrazine in a molar ratio of about 1 to 0.5 is characterized by a powder X-ray diffraction pattern comprising at least one peak selected from the group consisting 3.92, 7.89, 8.98, 10.70, 10.94, 11.42, 12.70, 13.42, 13.99, 14.55, 14.81, 15.49, 15.86, 16.14, 16.78, 17.18, 17.41, 17.66, 18.10, 18.71, 20.08, 20.47, 21.05, 21.22, 21.70, 22.23, 22.75, 23.28, 23.91, 24.81, 25.58, 27.04, 27.45, 28.57, 29.10, 29.88, and 30.62 (each degrees 2θ±0.2).

In certain embodiments, the solid form comprising cannabinol and tetramethylpyrazine in a molar ratio of about 1 to 0.5 is characterized by a powder X-ray diffraction pattern comprising at least two peaks selected from the group consisting of 3.92, 7.89, 8.98, 10.70, 10.94, 11.42, 12.70, 13.42, 13.99, 14.55, 14.81, 15.49, 15.86, 16.14, 16.78, 17.18, 17.41, 17.66, 18.10, 18.71, 20.08, 20.47, 21.05, 21.22, 21.70, 22.23, 22.75, 23.28, 23.91, 24.81, 25.58, 27.04, 27.45, 28.57, 29.10, 29.88, and 30.62 (each degrees 2θ±0.2).

In certain embodiments, the solid form comprising cannabinol and tetramethylpyrazine in a molar ratio of about 1 to 0.5 is characterized by a powder X-ray diffraction pattern comprising at least three peaks selected from the group consisting of 3.92, 7.89, 8.98, 10.70, 10.94, 11.42, 12.70, 13.42, 13.99, 14.55, 14.81, 15.49, 15.86, 16.14, 16.78, 17.18, 17.41, 17.66, 18.10, 18.71, 20.08, 20.47, 21.05, 21.22, 21.70, 22.23, 22.75, 23.28, 23.91, 24.81, 25.58, 27.04, 27.45, 28.57, 29.10, 29.88, and 30.62 (each degrees 2θ±0.2).

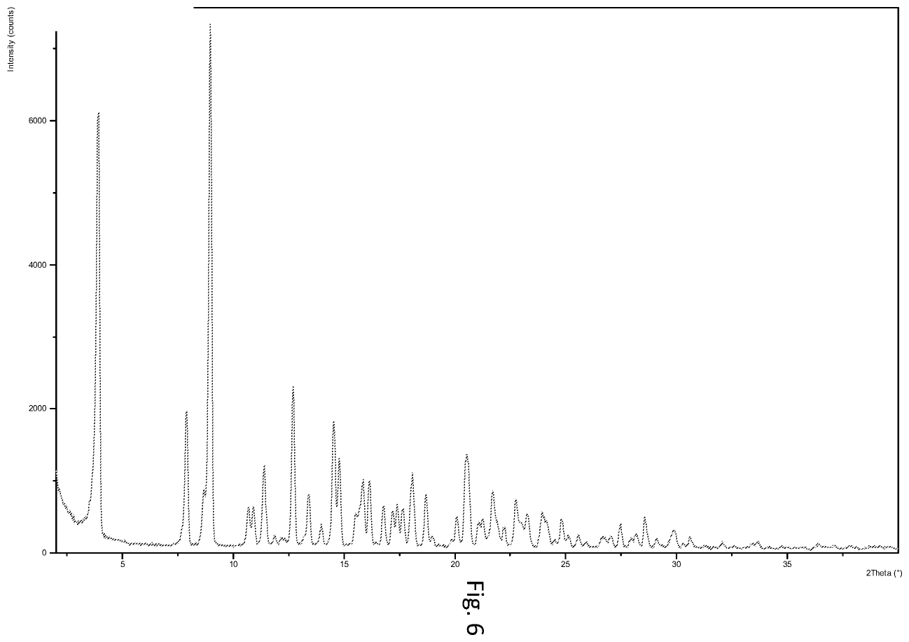

In certain embodiments, the solid form comprising cannabinol and tetramethylpyrazine in a molar ratio of about 1 to 0.5 is characterized by a powder X-ray diffraction pattern substantially as depicted in FIG. 6.

In certain embodiments, the solid form comprising cannabinol and tetramethylpyrazine in a molar ratio of about 1 to 0.5 is characterized by a DSC thermogram with a peak onset at about 72.8° C. and a peak maximum at about 74.3° C.

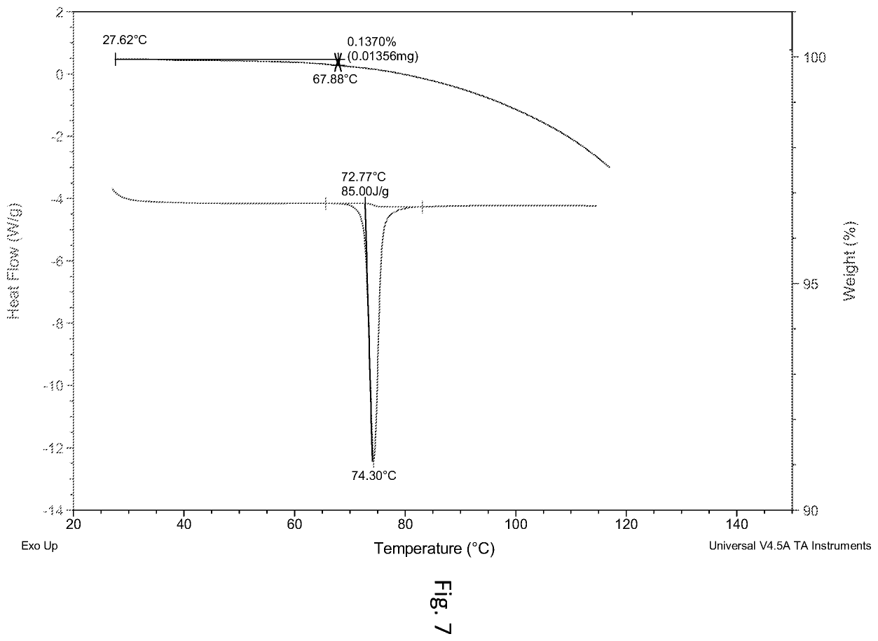

In certain embodiments, the solid form comprising cannabinol and tetramethylpyrazine in a molar ratio of about 1 to 0.5 is characterized by a DSC thermogram substantially as depicted in FIG. 7.

In certain embodiments, the solid form comprising cannabinol and tetramethylpyrazine in a molar ratio of about 1 to 0.5 is characterized by a Raman spectrum comprising at least one peak selected from the group consisting of 169, 228, 320, 365, 379, 408, 477, 509, 536, 549, 564, 581, 608, 664, 693, 714, 732, 774, 863, 883, 998, 1028, 1107, 1156, 1192, 1241, 1284, 1297, 1339, 1375, 1437, 1497, 1513, 1570, 1582, 1617, 2850, 2924, 2979, and 3039 (each cm⁻¹).

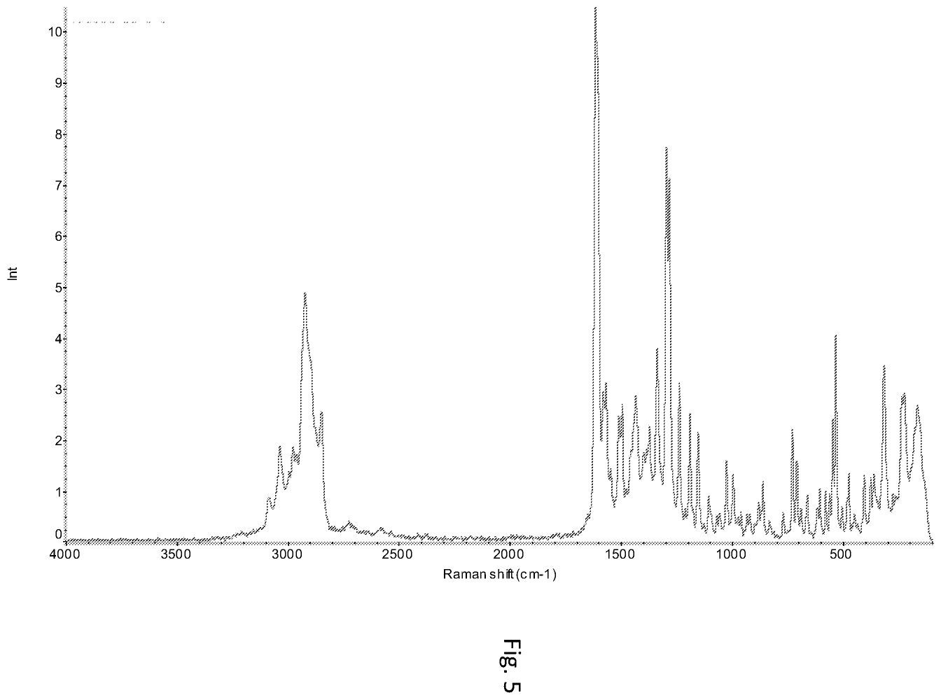

In certain embodiments, the solid form comprising cannabinol and tetramethylpyrazine in a molar ratio of about 1 to 0.5 is characterized by a Raman spectrum substantially as depicted in FIG. 5.

In certain embodiments, the solid form comprising cannabinol and tetramethylpyrazine in a molar ratio of about 1 to 0.5 is crystalline.

In certain embodiments, the solid form comprising cannabinol and tetramethylpyrazine in a molar ratio of about 1 to 0.5 is a cocrystal.

In certain embodiments, the solid form comprising cannabinol and tetramethylpyrazine in a molar ratio of about 1 to 0.5 is substantially free of solvent.

In certain embodiments, the solid form comprising cannabinol and tetramethylpyrazine in a molar ratio of about 1 to 0.5 is stable. In certain embodiments, the solid form comprising cannabinol and tetramethylpyrazine in a molar ratio of about 1 to 0.5 is stable up to 2 hr, 5 hr, 10, hr, 15 hr, 24 hr, 48 hr, 3 days, 5 days, 10 days, 12 days, 13 days, 15 days, 20 days, 30 days, 1 month, 2 months, 3 months, 6 months, 8 months, or 1 year.

In certain embodiments of the solid form, the molar ratio of cannabinol to tetramethylpyrazine is about 1 to 1.

In certain embodiments, the solid form comprising cannabinol and tetramethylpyrazine in a molar ratio of about 1 to 1 is characterized by a powder X-ray diffraction pattern comprising at least one peak selected from the group consisting of 6.91, 8.75, 10.29, 10.56, 11.13, 12.36, 13.76, 14.77, 15.24, 15.72, 16.41, 17.78, 18.80, 20.07, 20.48, 20.91, 21.76, 22.69, 23.24, 23.46, 23.95, 24.46, 24.86, 25.70, 27.32, 27.94, 28.45, 28.90, 31.45, 31.95, 35.57, and 36.71 (each degrees 2θ±0.2).

In certain embodiments, the solid form comprising cannabinol and tetramethylpyrazine in a molar ratio of about 1 to 1 is characterized by a powder X-ray diffraction pattern comprising at least two peaks selected from the group consisting of 6.91, 8.75, 10.29, 10.56, 11.13, 12.36, 13.76, 14.77, 15.24, 15.72, 16.41, 17.78, 18.80, 20.07, 20.48, 20.91, 21.76, 22.69, 23.24, 23.46, 23.95, 24.46, 24.86, 25.70, 27.32, 27.94, 28.45, 28.90, 31.45, 31.95, 35.57, and 36.71 (each degrees 2θ±0.2).

In certain embodiments, the solid form comprising cannabinol and tetramethylpyrazine in a molar ratio of about 1 to 1 is characterized by a powder X-ray diffraction pattern comprising at least three peaks selected from the group consisting of 6.91, 8.75, 10.29, 10.56, 11.13, 12.36, 13.76, 14.77, 15.24, 15.72, 16.41, 17.78, 18.80, 20.07, 20.48, 20.91, 21.76, 22.69, 23.24, 23.46, 23.95, 24.46, 24.86, 25.70, 27.32, 27.94, 28.45, 28.90, 31.45, 31.95, 35.57, and 36.71 (each degrees 2θ±0.2).

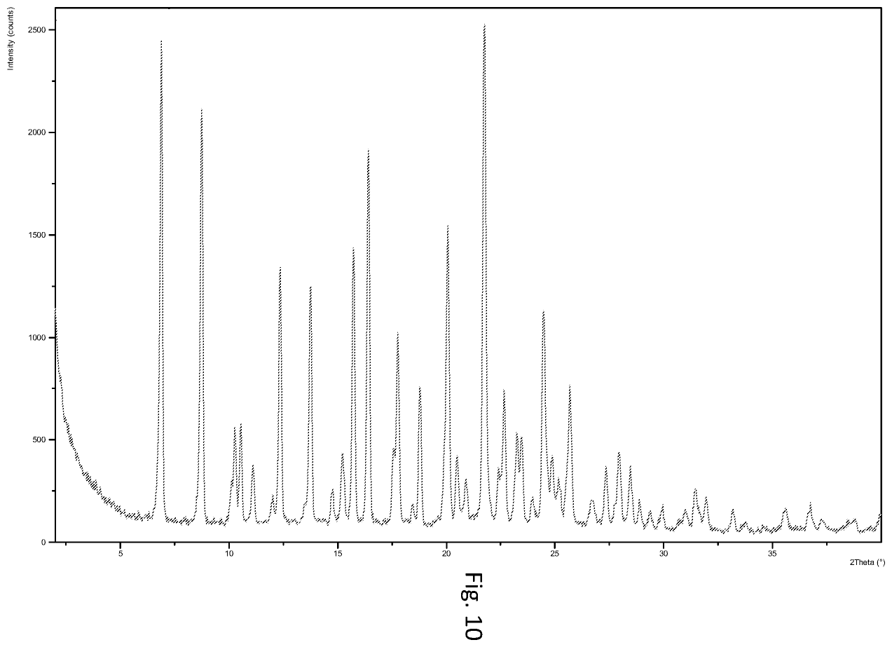

In certain embodiments, the solid form comprising cannabinol and tetramethylpyrazine in a molar ratio of about 1 to 1 is characterized by a powder X-ray diffraction pattern substantially as depicted in FIG. 10.

In certain embodiments, the solid form comprising cannabinol and tetramethylpyrazine in a molar ratio of about 1 to 1 is characterized by a DSC thermogram with a peak onset at about 76.3° C. and a peak maximum at about 77.8° C.

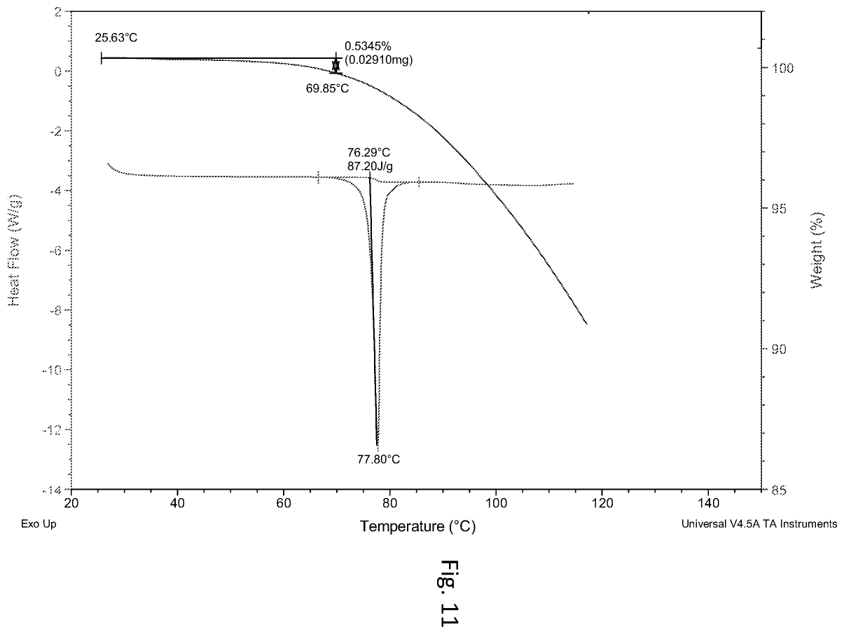

In certain embodiments, the solid form comprising cannabinol and tetramethylpyrazine in a molar ratio of about 1 to 1 is characterized by a DSC thermogram substantially as depicted in FIG. 11.

In certain embodiments, the solid form comprising cannabinol and tetramethylpyrazine in a molar ratio of about 1 to 1 is characterized by a Raman spectrum comprising at least one peak selected from the group consisting of 166, 229, 245, 264, 322, 343, 379, 409, 479, 507, 535, 583, 598, 626, 667, 703, 724, 862, 1027, 1101, 1158, 1191, 1225, 1240, 1284, 1300, 1342, 1385, 1440, 1506, 1549, 1573, 1584, 1607, 1618, 2856, 2925, 2969, 2987, and 3036 (each cm⁻¹).

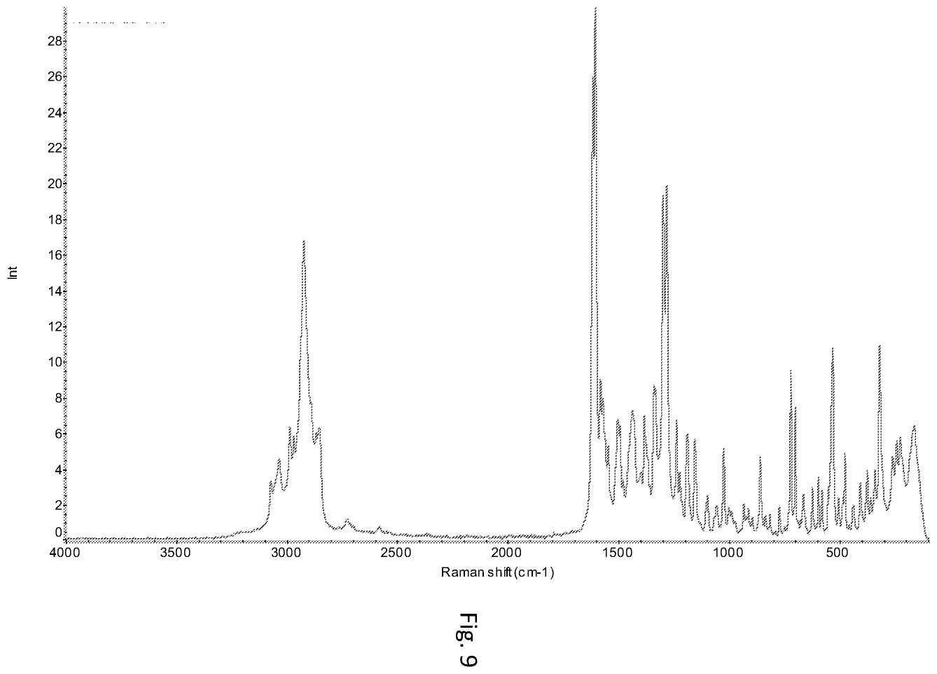

In certain embodiments, the solid form comprising cannabinol and tetramethylpyrazine in a molar ratio of about 1 to 1 is characterized by a Raman spectrum substantially as depicted in FIG. 9.

In certain embodiments, the solid form comprising cannabinol and tetramethylpyrazine in a molar ratio of about 1 to 1 is crystalline.

In certain embodiments, the solid form comprising cannabinol and tetramethylpyrazine in a molar ratio of about 1 to 1 is a cocrystal.

In certain embodiments, the solid form comprising cannabinol and tetramethylpyrazine in a molar ratio of about 1 to 1 is substantially free of solvent.

In certain embodiments, the solid form comprising cannabinol and tetramethylpyrazine in a molar ratio of about 1 to 1 is stable. In certain embodiments, the solid form comprising cannabinol and tetramethylpyrazine in a molar ratio of about 1 to 0.5 is stable up to 2 hr, 5 hr, 10, hr, 15 hr, 24 hr, 48 hr, 3 days, 5 days, 10 days, 12 days, 13 days, 15 days, 20 days, 30 days, 1 month, 2 months, 3 months, 6 months, 8 months, or 1 year.

In certain embodiments of the solid form, the cannabinoid is cannabinol and the coformer is L-proline.

In certain embodiments of the solid form, the molar ratio of cannabinol to L-proline is about 1 to 1.

In certain embodiments, the solid form comprising cannabinol and L-proline in a molar ratio of about 1 to 1 is characterized by a powder X-ray diffraction pattern comprising at least one peak selected from the group consisting of 4.72, 6.19, 8.21, 9.47, 10.98, 12.56, 13.75, 14.25, 16.03, 16.48, 17.15, 19.04, 19.85, 20.77, 21.50, 21.84, 23.09, 23.90, 24.80, 25.91, 26.62, 27.20, 37.69, and 38.26 (each degrees 2θ±0.2).

In certain embodiments, the solid form comprising cannabinol and L-proline in a molar ratio of about 1 to 1 is characterized by a powder X-ray diffraction pattern comprising at least two peaks selected from the group consisting of 4.72, 6.19, 8.21, 9.47, 10.98, 12.56, 13.75, 14.25, 16.03, 16.48, 17.15, 19.04, 19.85, 20.77, 21.50, 21.84, 23.09, 23.90, 24.80, 25.91, 26.62, 27.20, 37.69, and 38.26 (each degrees 2θ±0.2).

In certain embodiments, the solid form comprising cannabinol and L-proline in a molar ratio of about 1 to 1 is characterized by a powder X-ray diffraction pattern comprising at least three peaks selected from the group consisting of 4.72, 6.19, 8.21, 9.47, 10.98, 12.56, 13.75, 14.25, 16.03, 16.48, 17.15, 19.04, 19.85, 20.77, 21.50, 21.84, 23.09, 23.90, 24.80, 25.91, 26.62, 27.20, 37.69, and 38.26 (each degrees 2θ±0.2).

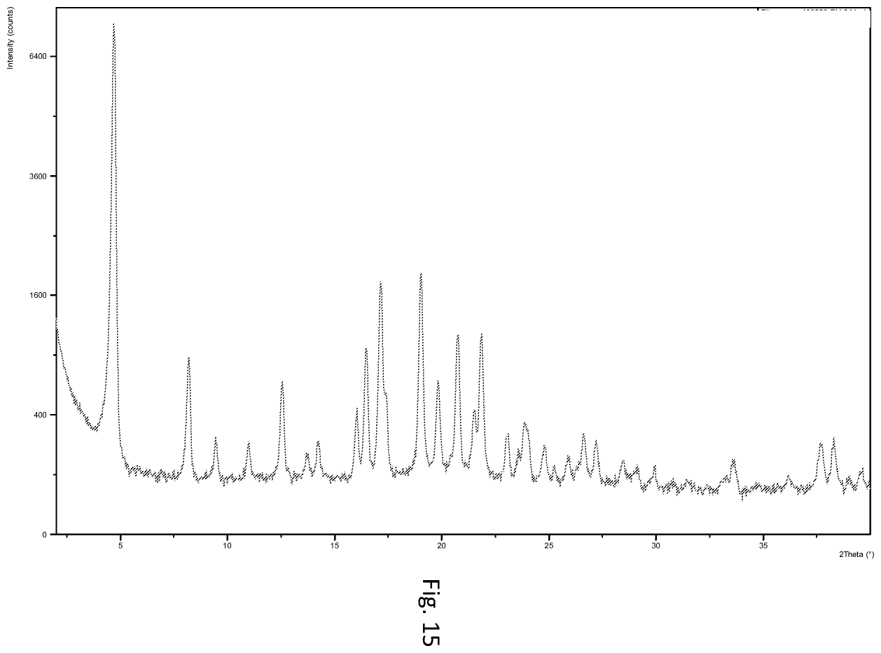

In certain embodiments, the solid form comprising cannabinol and L-proline in a molar ratio of about 1 to 1 is characterized by a powder X-ray diffraction pattern substantially as depicted in FIG. 15.

In certain embodiments, the solid form comprising cannabinol and L-proline in a molar ratio of about 1 to 1 is characterized by a DSC thermogram with a peak onset at about 120.7° C. and a peak maximum at about 124.3° C.

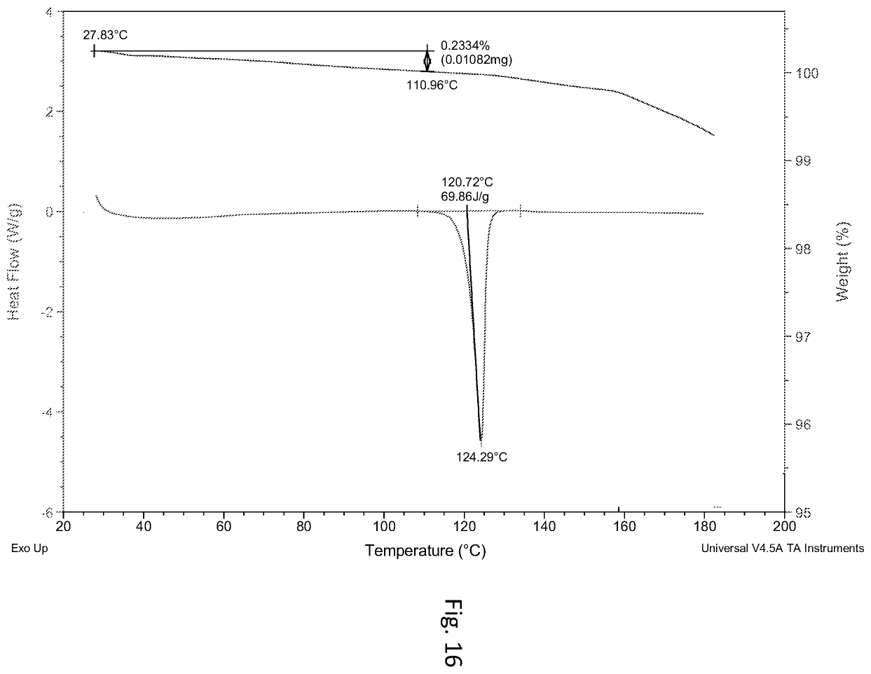

In certain embodiments, the solid form comprising cannabinol and L-proline in a molar ratio of about 1 to 1 is characterized by a DSC thermogram substantially as depicted in FIG. 16.

In certain embodiments, the solid form comprising cannabinol and L-proline in a molar ratio of about 1 to 1 is characterized by a Raman spectrum comprising at least one peak selected from the group consisting of 163, 240, 324, 362, 408, 486, 509, 530, 551, 580, 621, 671, 696, 711, 773, 841, 863, 915, 935, 995, 1027, 1110, 1157, 1194, 1236, 1297, 1330, 1400, 1444, 1496, 1509, 1583, 1615, 2885, 2923, 2972, and 3020 (each cm⁻¹).

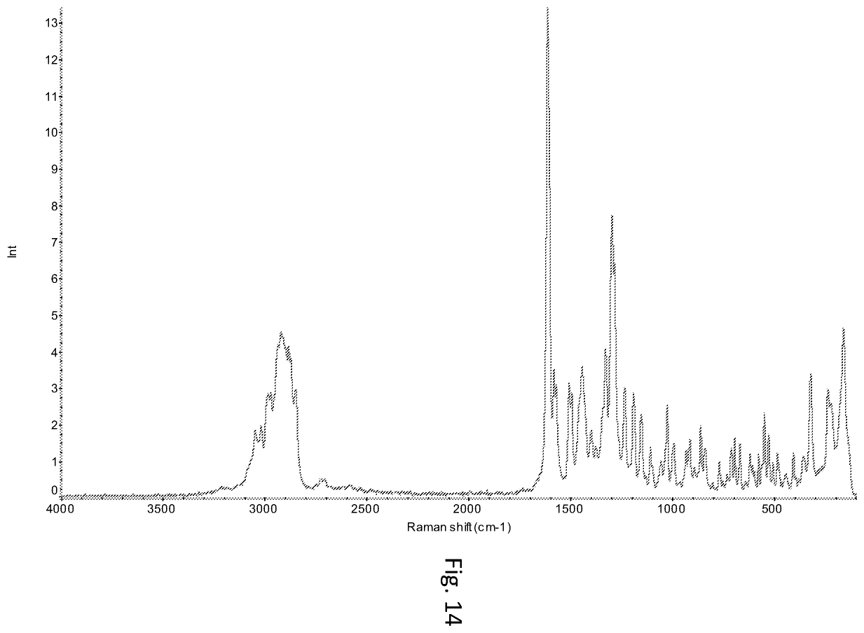

In certain embodiments, the solid form comprising cannabinol and L-proline in a molar ratio of about 1 to 1 is characterized by a Raman spectrum substantially as depicted in FIG. 14.

In certain embodiments, the solid form comprising cannabinol and L-proline in a molar ratio of about 1 to 1 is crystalline.

In certain embodiments, the solid form comprising cannabinol and L-proline in a molar ratio of about 1 to 1 is a cocrystal.

In certain embodiments, the solid form comprising cannabinol and L-proline in a molar ratio of about 1 to 1 is substantially free of solvent.

In certain embodiments, the solid form comprising cannabinol and L-proline in a molar ratio of about 1 to 1 is stable. In certain embodiments, the solid form comprising cannabinol to L-proline in a molar ratio of about 1 to 1 is stable up to 2 hr, 5 hr, 10, hr, 15 hr, 24 hr, 48 hr, 3 days, 5 days, 10 days, 12 days, 13 days, 15 days, 20 days, 30 days, 1 month, 2 months, 3 months, 6 months, 8 months, or 1 year.

In certain embodiments of the solid form, the cannabinoid is cannabinol and the coformer is D-proline.

In certain embodiments of the solid form, the molar ratio of cannabinol to D-proline is about 1 to 1.

In certain embodiments, the solid form comprising cannabinol and D-proline in a molar ratio of about 1 to 1 is characterized by a powder X-ray diffraction pattern comprising at least one peak selected from the group consisting of 4.70, 8.18, 9.44, 10.95, 12.53, 15.99, 16.44, 17.11, 19.01, 19.82, 20.74, 21.46, 21.81, 23.01, 23.93, 24.77, 26.55, 27.14, 37.64, and 38.24 (each degrees 2θ±0.2).

In certain embodiments, the solid form comprising cannabinol and D-proline in a molar ratio of about 1 to 1 is characterized by a powder X-ray diffraction pattern comprising at least two peaks selected from the group consisting of 4.70, 8.18, 9.44, 10.95, 12.53, 15.99, 16.44, 17.11, 19.01, 19.82, 20.74, 21.46, 21.81, 23.01, 23.93, 24.77, 26.55, 27.14, 37.64, and 38.24 (each degrees 2θ±0.2).

In certain embodiments, the solid form comprising cannabinol and D-proline in a molar ratio of about 1 to 1 is characterized by a powder X-ray diffraction pattern comprising at least three peaks selected from the group consisting of 4.70, 8.18, 9.44, 10.95, 12.53, 15.99, 16.44, 17.11, 19.01, 19.82, 20.74, 21.46, 21.81, 23.01, 23.93, 24.77, 26.55, 27.14, 37.64, and 38.24 (each degrees 2θ±0.2).

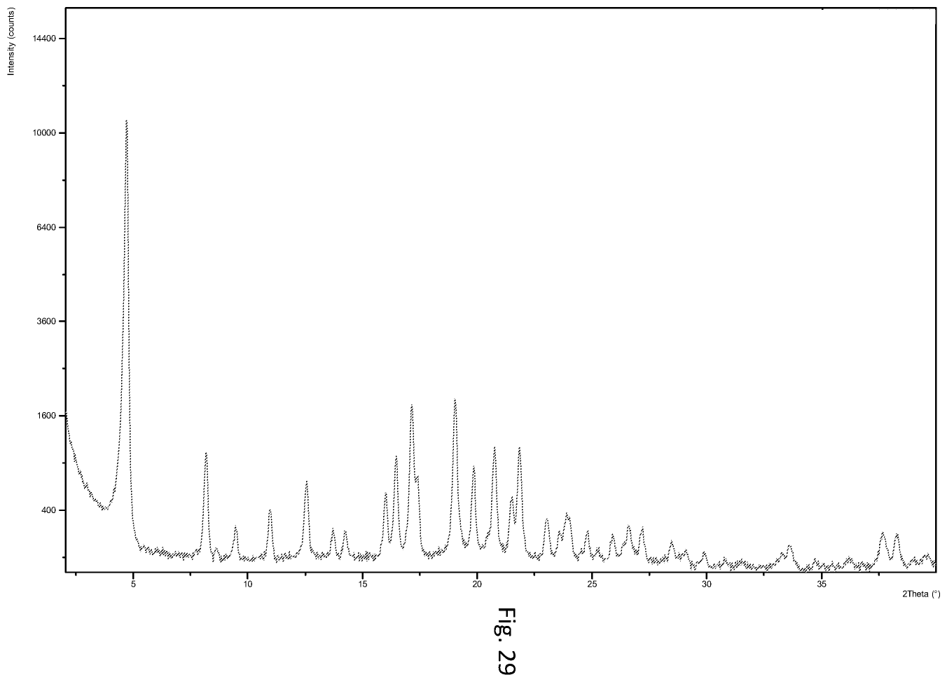

In certain embodiments, the solid form comprising cannabinol and D-proline in a molar ratio of about 1 to 1 is characterized by a powder X-ray diffraction pattern substantially as depicted in FIG. 29.

In certain embodiments, the solid form comprising cannabinol and D-proline in a molar ratio of about 1 to 1 is characterized by a DSC thermogram with a peak onset at about 119.5° C. and a peak maximum at about 125.3° C.

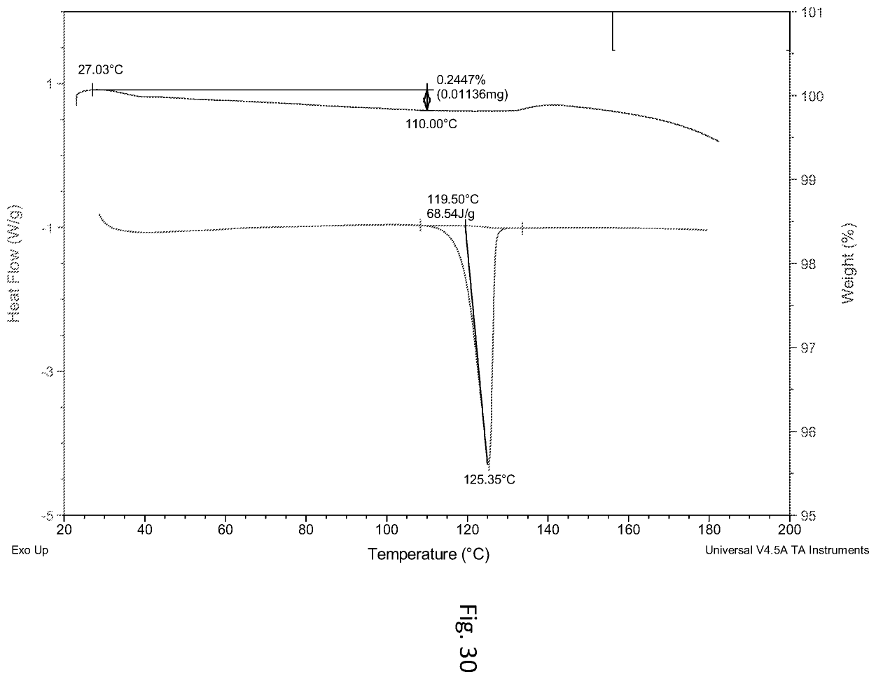

In certain embodiments, the solid form comprising cannabinol and D-proline in a molar ratio of about 1 to 1 is characterized by a DSC thermogram substantially as depicted in FIG. 30.

In certain embodiments, the solid form comprising cannabinol and D-proline in a molar ratio of about 1 to 1 is characterized by a Raman spectrum comprising at least one peak selected from the group consisting of 163, 239, 323, 362, 408, 486, 508, 531, 551, 580, 621, 671, 696, 711, 773, 841, 863, 915, 995, 1027, 1110, 1157, 1193, 1236, 1298, 1329, 1399, 1443, 1508, 1583, 1615, 2924, 2985, and 3021 (each cm⁻¹).

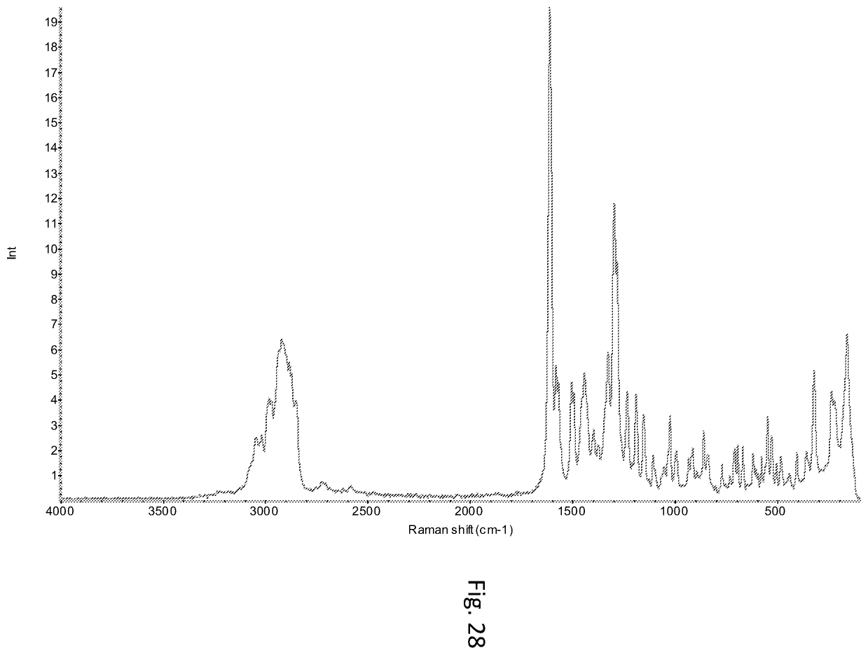

In certain embodiments, the solid form comprising cannabinol and D-proline in a molar ratio of about 1 to 1 is characterized by a Raman spectrum substantially as depicted in FIG. 28.

In certain embodiments, the solid form comprising cannabinol and D-proline in a molar ratio of about 1 to 1 is crystalline.

In certain embodiments, the solid form comprising cannabinol and D-proline in a molar ratio of about 1 to 1 is a cocrystal.

In certain embodiments, the solid form comprising cannabinol and D-proline in a molar ratio of about 1 to 1 is substantially free of solvent.

In certain embodiments, the solid form comprising cannabinol and D-proline in a molar ratio of about 1 to 1 is stable.

In certain embodiments, the subject matter described herein is directed to a solid form comprising a solvated cocrystal of cannabinol and a coformer.

In certain embodiments of the solvated cocrystal comprising cannabinol and a coformer, the cannabinol and coformer are present in a molar ratio of 1:1 to about 1:1.2.

In certain embodiments of the solvated cocrystal comprising cannabinol and a coformer, the coformer is L-proline or D-proline.

In certain embodiments of the solvated cocrystal comprising cannabinol and a coformer, the cocrystal is an isooctane or petroleum ether solvate.

In certain embodiments of the solvated cocrystal comprising cannabinol and L-proline, the cocrystal is an isooctane solvate.

In certain embodiments of the solvated cocrystal comprising cannabinol and L-proline, the cocrystal is an isooctane solvate, wherein the cannabinol and coformer are present in a molar ratio of about 1:1.1.

In certain embodiments of the isooctane solvated cocrystal comprising cannabinol and L-proline, the cocrystal is characterized by a powder X-ray diffraction pattern comprising at least one peak selected from the group consisting of 5.09, 10.23, 11.45, 12.52, 14.49, 15.40, 16.22, 17.77, 18.52, 19.17, 20.56, 21.82, 22.38, 23.05, 24.13, 25.21, 25.82, and 27.32 (each degrees 2θ±0.2).

In certain embodiments of the isooctane solvated cocrystal comprising cannabinol and L-proline, the cocrystal is characterized by a powder X-ray diffraction pattern comprising at least two peaks selected from the group consisting of 5.09, 10.23, 11.45, 12.52, 14.49, 15.40, 16.22, 17.77, 18.52, 19.17, 20.56, 21.82, 22.38, 23.05, 24.13, 25.21, 25.82, and 27.32 (each degrees 2θ±0.2).

In certain embodiments of the isooctane solvated cocrystal comprising cannabinol and L-proline, the cocrystal is characterized by a powder X-ray diffraction pattern comprising at least three peaks selected from the group consisting of 5.09, 10.23, 11.45, 12.52, 14.49, 15.40, 16.22, 17.77, 18.52, 19.17, 20.56, 21.82, 22.38, 23.05, 24.13, 25.21, 25.82, and 27.32 (each degrees 2θ±0.2).

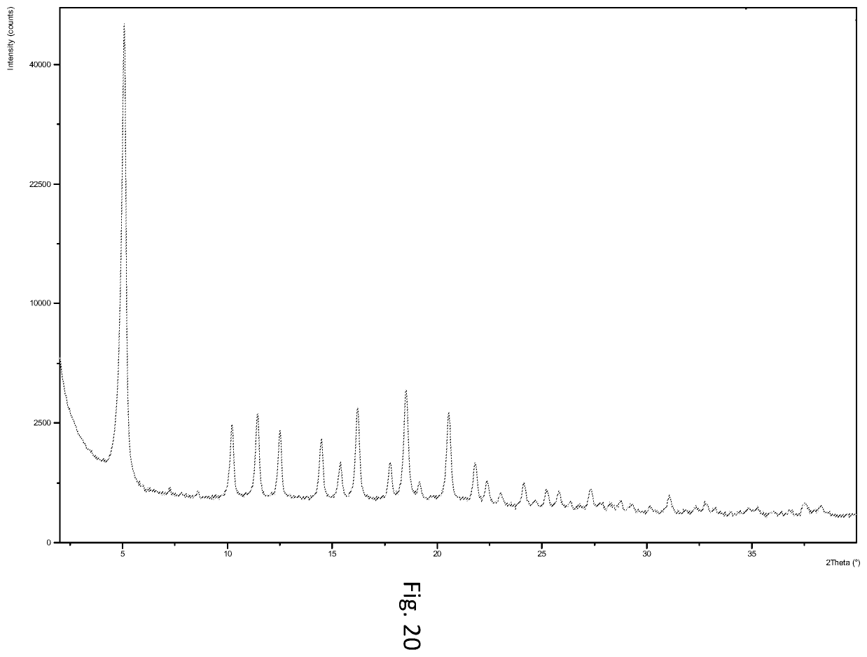

In certain embodiments of the isooctane solvated cocrystal comprising cannabinol and L-proline, the cocrystal is characterized by a powder X-ray diffraction pattern substantially as depicted in FIG. 20.

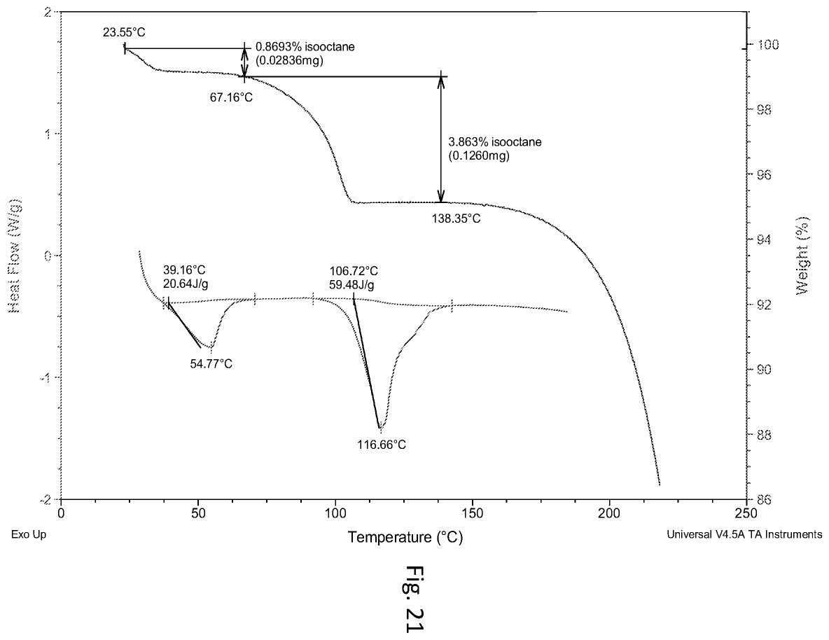

In certain embodiments of the isooctane solvated cocrystal comprising cannabinol and L-proline, the cocrystal is characterized by a DSC thermogram substantially as depicted in FIG. 21.

In certain embodiments of the isooctane solvated cocrystal comprising cannabinol and L-proline, the cocrystal is characterized by a Raman spectrum comprising at least one peak selected from the group consisting of 163, 222, 320, 353, 410, 489, 533, 548, 583, 670, 713, 864, 902, 1030, 1155, 1194, 1240, 1284, 1303, 1333, 1376, 1405, 1438, 1496, 1512, 1573, 1587, 1610, 1619, 2850, 2922, 2988, and 3049 (each cm⁻¹).

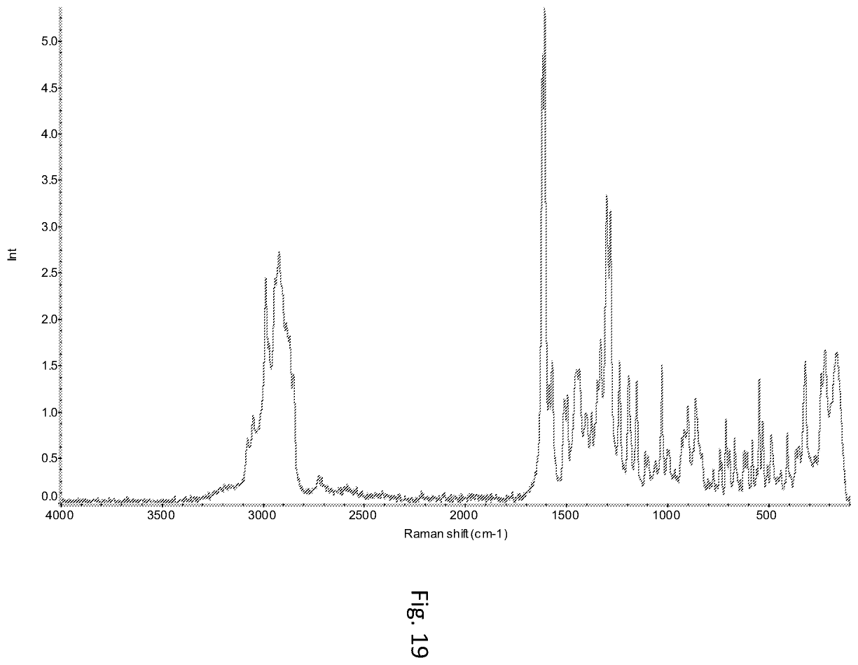

In certain embodiments of the isooctane solvated cocrystal comprising cannabinol and L-proline, the cocrystal is characterized by a Raman spectrum substantially as depicted in FIG. 19.

In certain embodiments of the solvated cocrystal comprising cannabinol and L-proline, the cocrystal is a petroleum ether solvate.

In certain embodiments of the petroleum ether solvated cocrystal comprising cannabinol and L-proline, the cocrystal is characterized by a powder X-ray diffraction pattern comprising at least one peak selected from the group consisting of 5.09, 10.25, 10.81, 11.45, 12.53, 14.49, 15.17, 16.21, 18.07, 18.51, 19.60, 20.55, 21.82, 22.80, 24.80, 25.83, 27.18, 30.61, and 32.18 (each degrees 2θ±0.2).

In certain embodiments of the petroleum ether solvated cocrystal comprising cannabinol and L-proline, the cocrystal is characterized by a powder X-ray diffraction pattern comprising at least two peaks selected from the group consisting of 5.09, 10.25, 10.81, 11.45, 12.53, 14.49, 15.17, 16.21, 18.07, 18.51, 19.60, 20.55, 21.82, 22.80, 24.80, 25.83, 27.18, 30.61, and 32.18 (each degrees 2θ±0.2).

In certain embodiments of the petroleum ether solvated cocrystal comprising cannabinol and L-proline, the cocrystal is characterized by a powder X-ray diffraction pattern comprising at least three peaks selected from the group consisting of 5.09, 10.25, 10.81, 11.45, 12.53, 14.49, 15.17, 16.21, 18.07, 18.51, 19.60, 20.55, 21.82, 22.80, 24.80, 25.83, 27.18, 30.61, and 32.18 (each degrees 2θ±0.2).

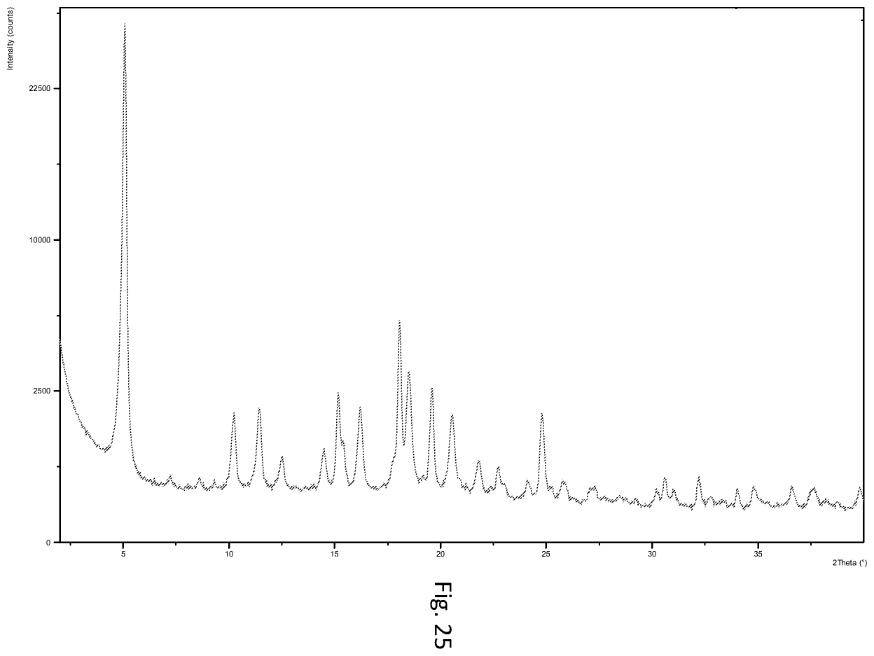

In certain embodiments of the petroleum ether solvated cocrystal comprising cannabinol and L-proline, the cocrystal is characterized by a powder X-ray diffraction pattern substantially as depicted in FIG. 25.

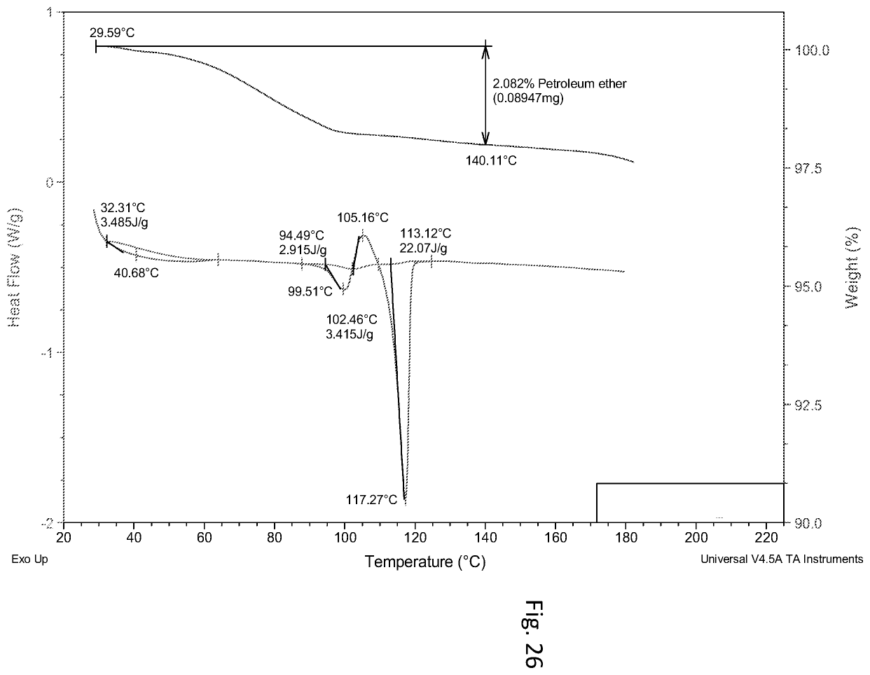

In certain embodiments of the petroleum ether solvated cocrystal comprising cannabinol and L-proline, the cocrystal is characterized by a DSC thermogram substantially as depicted in FIG. 26.

In certain embodiments of the petroleum ether solvated cocrystal comprising cannabinol and L-proline, the cocrystal is characterized by a Raman spectrum comprising at least one peak selected from the group consisting of 169, 224, 321, 410, 448, 489, 531, 548, 582, 606, 623, 669, 694, 712, 842, 863, 899, 919, 951, 993, 1030, 1056, 1155, 1193, 1239, 1284, 1302, 1333, 1376, 1404, 1444, 1511, 1572, 1585, 1609, 1618, 2931, 2984, and 3001 (each cm⁻¹).

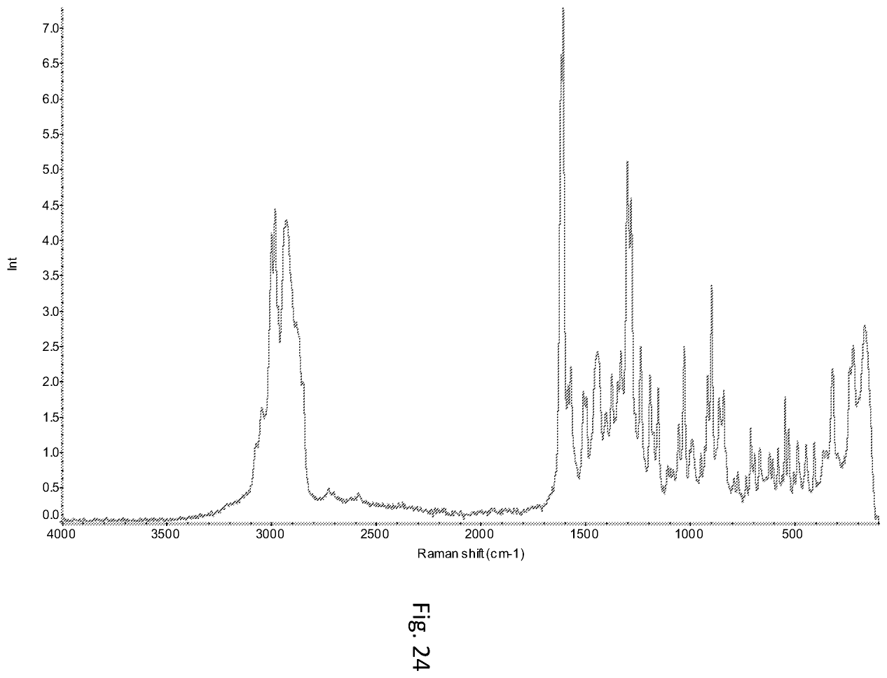

In certain embodiments of the petroleum ether solvated cocrystal comprising cannabinol and L-proline, the cocrystal is characterized by a Raman spectrum substantially as depicted in FIG. 24.

In certain embodiments of the solvated cocrystal comprising cannabinol and D-proline, the cocrystal is an isooctane solvate.

In certain embodiments of the solvated cocrystal comprising cannabinol and D-proline, the cocrystal is an isooctane solvate, wherein the wherein the cannabinol and coformer are present in a molar ratio of about 1:1.2.

In certain embodiments of the isooctane solvated cocrystal comprising cannabinol and D-proline, the cocrystal is characterized by a powder X-ray diffraction pattern comprising at least one peak selected from the group consisting of 5.13, 8.65, 10.28, 11.50, 12.58, 14.54, 15.46, 16.28, 17.84, 18.58, 19.22, 20.63, 21.89, 22.47, 24.14, 25.31, 25.87, 27.41, and 38.24 (each degrees 2θ±0.2).

In certain embodiments of the isooctane solvated cocrystal comprising cannabinol and D-proline, the cocrystal is characterized by a powder X-ray diffraction pattern comprising at least two peaks selected from the group consisting of 5.13, 8.65, 10.28, 11.50, 12.58, 14.54, 15.46, 16.28, 17.84, 18.58, 19.22, 20.63, 21.89, 22.47, 24.14, 25.31, 25.87, 27.41, and 38.24 (each degrees 2θ±0.2).

In certain embodiments of the isooctane solvated cocrystal comprising cannabinol and D-proline, the cocrystal is characterized by a powder X-ray diffraction pattern comprising at least three peaks selected from the group consisting of 5.13, 8.65, 10.28, 11.50, 12.58, 14.54, 15.46, 16.28, 17.84, 18.58, 19.22, 20.63, 21.89, 22.47, 24.14, 25.31, 25.87, 27.41, and 38.24 (each degrees 2θ±0.2).

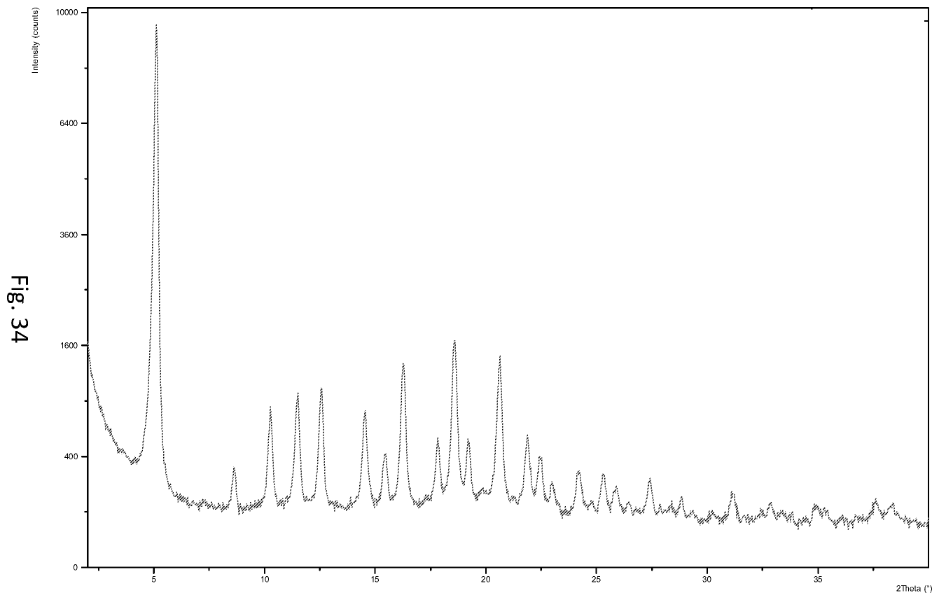

In certain embodiments of the isooctane solvated cocrystal comprising cannabinol and D-proline, the cocrystal is characterized by a powder X-ray diffraction pattern substantially as depicted in FIG. 34.

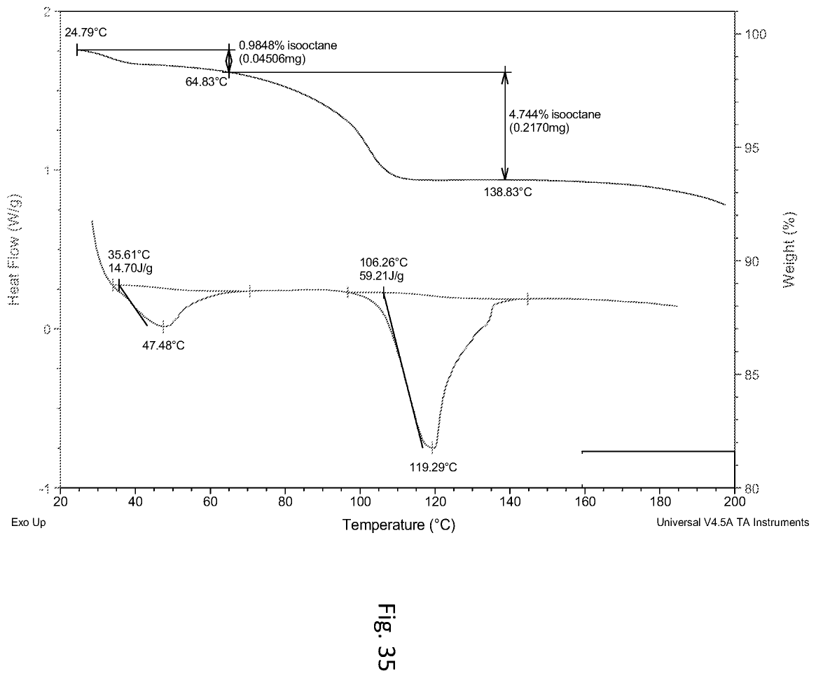

In certain embodiments of the isooctane solvated cocrystal comprising cannabinol and D-proline, the cocrystal is characterized by a DSC thermogram substantially as depicted in FIG. 35.

In certain embodiments of the isooctane solvated cocrystal comprising cannabinol and D-proline, the cocrystal is characterized by a Raman spectrum comprising at least one peak selected from the group consisting of 168, 224, 242, 320, 350, 409, 489, 532, 548, 583, 623, 670, 712, 743, 830, 863, 900, 921, 931, 1029, 1154, 1194, 1239, 1283, 1303, 1333, 1348, 1376, 1404, 1437, 1496, 1512, 1572, 1587, 1609, 1619, 2869, 2922, 2988, and 3049 (each cm⁻¹).

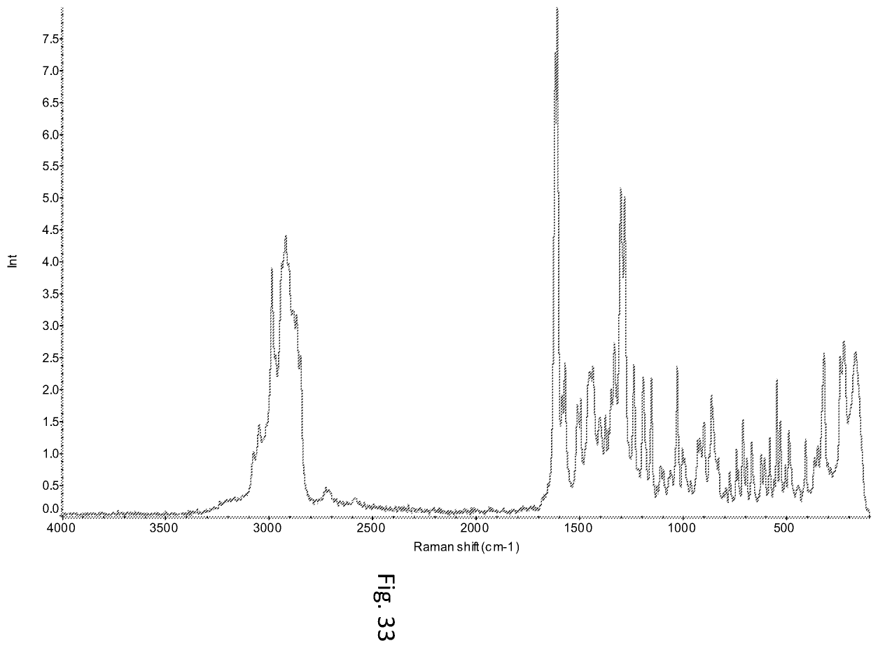

In certain embodiments of the isooctane solvated cocrystal comprising cannabinol and D-proline, the cocrystal is characterized by a Raman spectrum substantially as depicted in FIG. 33.

In certain embodiments of the solvated cocrystal comprising cannabinol and D-proline, the cocrystal is a petroleum ether solvate.

In certain embodiments of the petroleum ether solvated cocrystal comprising cannabinol and D-proline, the cocrystal is characterized by a powder X-ray diffraction pattern comprising at least one peak selected from the group consisting of 5.12, 8.64, 10.26, 11.48, 12.54, 14.52, 14.83, 15.42, 16.24, 17.79, 18.55, 19.18, 20.58, 21.83, 22.99, 24.17, 25.27, 25.80, and 27.33 (each degrees 2θ±0.2).

In certain embodiments of the petroleum ether solvated cocrystal comprising cannabinol and D-proline, the cocrystal is characterized by a powder X-ray diffraction pattern comprising at least two peaks selected from the group consisting of 5.12, 8.64, 10.26, 11.48, 12.54, 14.52, 14.83, 15.42, 16.24, 17.79, 18.55, 19.18, 20.58, 21.83, 22.99, 24.17, 25.27, 25.80, and 27.33 (each degrees 2θ±0.2).

In certain embodiments of the petroleum ether solvated cocrystal comprising cannabinol and D-proline, the cocrystal is characterized by a powder X-ray diffraction pattern comprising at least three peaks selected from the group consisting of 5.12, 8.64, 10.26, 11.48, 12.54, 14.52, 14.83, 15.42, 16.24, 17.79, 18.55, 19.18, 20.58, 21.83, 22.99, 24.17, 25.27, 25.80, and 27.33 (each degrees 2θ±0.2).

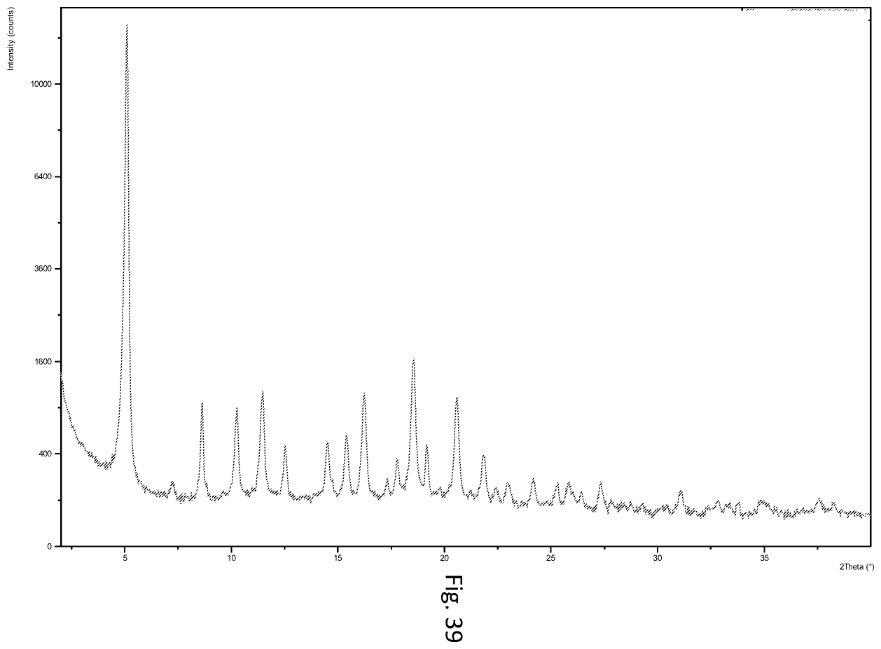

In certain embodiments of the petroleum ether solvated cocrystal comprising cannabinol and D-proline, the cocrystal is characterized by a powder X-ray diffraction pattern substantially as depicted in FIG. 39.

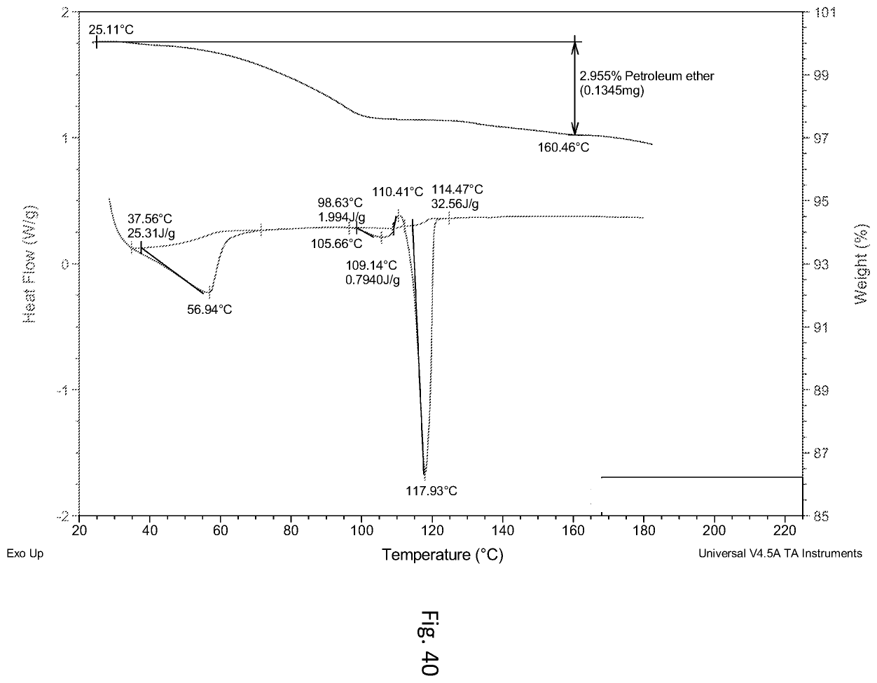

In certain embodiments of the petroleum ether solvated cocrystal comprising cannabinol and D-proline, the cocrystal is characterized by a DSC thermogram substantially as depicted in FIG. 40.

In certain embodiments of the petroleum ether solvated cocrystal comprising cannabinol and D-proline, the cocrystal is characterized by a Raman spectrum comprising at least one peak selected from the group consisting of 164, 224, 320, 409, 489, 532, 548, 583, 623, 670, 712, 863, 903, 1029, 1154, 1193, 1239, 1283, 1302, 1332, 1376, 1404, 1437, 1510, 1572, 1585, 1609, 1618, 2921, and 2986 (each cm⁻¹).

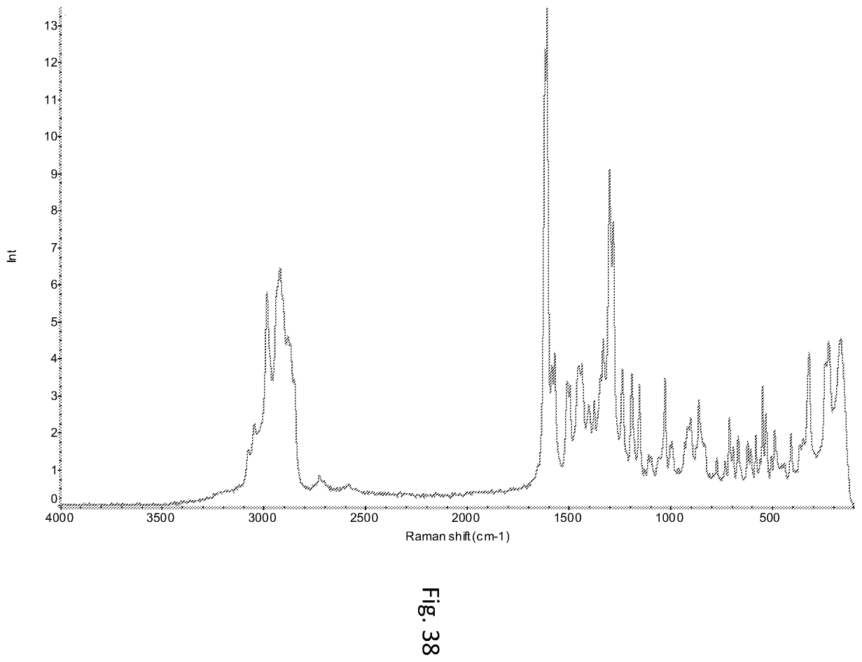

In certain embodiments of the petroleum ether solvated cocrystal comprising cannabinol and D-proline, the cocrystal is characterized by a Raman spectrum substantially as depicted in FIG. 38.

### III. Methods of Preparing Cocrystals

In certain embodiments, the subject matter disclosed herein is directed to a method of preparing a solid form comprising a coformer and a cannabinoid, starting from a cannabinoid oil selected from the group consisting of cannabinol and tetrahydrocannabinol, the method comprising,
contacting the cannabinoid with the coformer and a solvent to prepare a suspension;
seeding the suspension with a seed cocrystal;
heating the suspension at a first temperature with stirring; and
separating a solid material from the suspension to form a cocrystal comprising the cannabinoid and the coformer.

In certain embodiments, the cannabinoid oil is cannabinol. The cannabinol oil starting material can either be obtained from the cannabis plant or prepared synthetically in the laboratory. In certain embodiments, synthetic cannabinol can be prepared in accordance with Scheme 1:

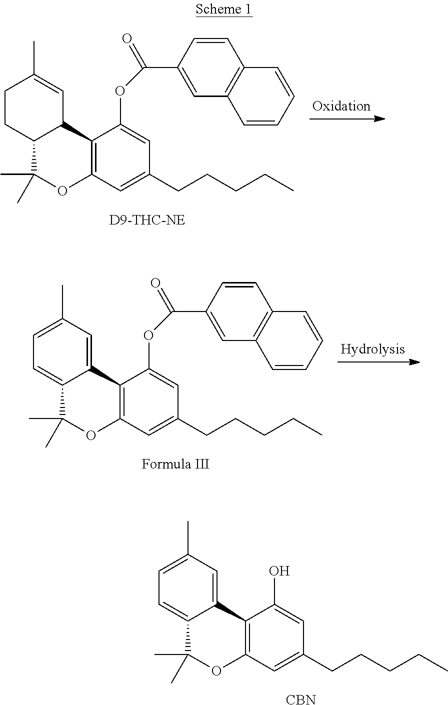

As shown in Scheme 1, D9-THC-NE (D9-THC-Naphthoylester) undergoes oxidation, optionally in the presence of a first solvent, to prepare a compound of Formula III. The compound of Formula III is then contacted with a base in the presence of a second solvent to prepare cannabinol. In certain embodiments, the oxidant is I₂ or sulfur. In certain embodiments, the first solvent is selected from the group consisting of toluene, acetone, methanol or other C₁₋₄ alcohol, 2-butanone, ethyl acetate, 1-4-dioxane, diethyl ether, tert-butyl methyl ether, tetrahydrofuran, dichloromethane, chloroform, heptane, isopropyl acetate, isooctane, n-decane, and anisole, and mixtures thereof. In certain embodiments, the base is a metal hydroxide, for example, LiOH, KOH, NaOH, Sr(OH)₂, Ba(OH)₂, Ca(OH)₂, or RbOH. In certain embodiments, the base is an alkali metal hydroxide, which is comprised of an alkali metal cation and a hydroxide anion. In certain embodiments, the second solvent is selected from the group consisting of THF, methanol, methyl-THF, ethanol, isopropanol, butanol or other C₁₋₄ alcohol, DMF and water, and mixtures thereof.

In certain embodiments of the method for preparing a solid form comprising a coformer and a cannabinoid, the coformer is selected from the group consisting of potassium, calcium, carnitine, aspartame, L-proline, D-proline, L-Arginine, L-Lysine, betaine, tetramethylpyrazine, 1H-Imidazole, nicotinic acid, saccharin, urea, and nicotinamide. In certain embodiments, the coformer is selected from the group consisting of tetramethylpyrazine, L-proline, and D-proline.

In certain embodiments of the method for preparing a solid form comprising a coformer and a cannabinoid, the ratio of molar equivalents of cannabinoid to coformer is about 1 to about 0.5, about 1 to about 2.0, about 1 to about 1, about 1 to about 0.8, about 0.2 to about 10, about 0.3 to about 8, about 0.5 to about 6, about 0.7 to about 5, about 0.8 to about 4, about 1 to about 4, about 1 to about 8, about 0.9 to about 3, about 0.9 to about 1, about 1 to about 2, or about 0.8 to about 1.

In certain embodiments of the method for preparing a solid form comprising a coformer and a cannabinoid, contacting said cannabinoid with said coformer and a solvent to prepare a suspension is for a period of about 5 minutes to about 96 hours; or about 10 minutes to about 84 hours; or about 20 minutes to about 72 hours; or about 48 hours to about 72 hours; or about 30 minutes to about 6 hours; or about 45 minutes to about 2 hours.

In certain embodiments of the method for preparing a solid form comprising a coformer and a cannabinoid, the solvent is selected from the group consisting of isooctane, petroleum ether, methanol, isopropanol, acetonitrile, acetone, tetrahydrofuran, 1,4-dioxane, ethyl acetate, 4-methyl-2-pentanone, dichloromethane, methyl t-butyl ether, and toluene. In certain embodiments, the solvent is isooctane or petroleum ether.

In certain embodiments of the method for preparing a solid form comprising a coformer and a cannabinoid, the amount of solvent present is between about 0.3 to about 50 volumes. In certain embodiments of the method for preparing a solid form comprising a coformer and a cannabinoid, the amount of solvent present is between about 2 to about 89 volumes. In certain embodiments, the amount of solvent present is about 0.5, 0.6, 0.7, 0.8, 0.9, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, or 30 volumes.

In certain embodiments, the contacting of the cannabinoid with the coformer and a solvent is at a temperature between about 20 and about 100° C. In certain embodiments, the contacting of the cannabinoid with the coformer and a solvent is at a temperature between about 5 and about 40° C. In certain embodiments, the contacting of the cannabinoid with the coformer and a solvent is at a temperature of about 25° C.

In certain embodiments, heating the suspension at a first temperature with stirring proceeds for about 5 seconds to about 6 days, about 5 minutes to about 96 hours; or about 10 minutes to about 84 hours; or about 20 minutes to about 72 hours; or about 48 hours to about 72 hours; or about 30 minutes to about 6 hours; or about 45 minutes to about 2 hours.

In certain embodiments, of the method for preparing a solid form comprising a coformer and a cannabinoid, the first temperature is between about 20° C. to about 400° C. In certain embodiments, the first temperature is about 20° C., 21° C., 22° C., 23° C., 24° C., 25° C., 26° C., 27° C., 28° C., 29° C., or 30° C. In certain embodiments, the first temperature comprises a series of temperatures in a temperature cycle between about 45° C. and 3° C., 60° C. and 0° C., or 40° C. and 5° C. In the cycle, the temperature is maintained for about 1 hour at each temperature.

In certain embodiments of the method for preparing a solid form comprising a coformer and a cannabinoid, the method further comprises adding more solvent to the suspension during stirring. In certain embodiments, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 additional volumes of solvent are added to the suspension during stirring.

In certain embodiments of the method for preparing a solid form comprising a coformer and a cannabinoid, the solid form is crystalline.

In certain embodiments of the method for preparing a solid form comprising a coformer and a cannabinoid, the solid form is a cocrystal.

In certain embodiments of the method for preparing a solid form comprising a coformer and a cannabinoid, the cocrystal is characterized by a powder X-ray diffraction pattern substantially as depicted in any one of FIG. 6, 10, 15, 20, 25, 29, 34, or 39.

### IV. Indications and Methods of Treatment

It is contemplated that the cannabinoid cocrystal compositions disclosed herein may be used as analgesics, antibiotics, and/or to treat a disease responsive to immunosuppressive and anti-inflammatory properties of the cannabinoid cocrystal compositions or responsive to the cannabinoid cocrystal compositions' affinity to cannabinoid receptors. The diseases may include, but are not limited to, emesis, pain, epilepsy, Alzheimer's disease, Huntington's disease, Tourette's syndrome, glaucoma, osteoporosis, schizophrenia, cancer, obesity, autoimmune diseases, diabetic complications, infections against methicillin-resistant Staphylococcus aureus, nausea, depression, anxiety, Hypoxia-ischemia injuries, psychosis, and inflammatory diseases.

Autoimmune diseases include, for example, Acquired Immunodeficiency Syndrome (AIDS), alopecia areata, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigold, cold agglutinin disease, crest syndrome, Crohn's disease, Degos' disease, dermatomyositis juvenile, discoid lupus, essential mixed cryoglobulinemia, fibromyalgia-fibromyositis, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, Meniere's disease, mixed connective tissue disease, multiple sclerosis, myasthenia gravis, Parkinson's disease, pernacious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, Reiter's syndrome, rheumatic fever, rheumatoid arthritis, sarcoidosis, scleroderma (progressive systemic sclerosis (PSS), also known as systemic sclerosis (SS)), Sjögren's syndrome, stiff-man syndrome, systemic lupus erythematosus, Takayasu arteritis, temporal arteritis/giant cell arteritis, ulcerative colitis, uveitis, vitiligo and Wegener's granulomatosis.

Inflammatory disorders, include, for example, chronic and acute inflammatory disorders. Examples of inflammatory disorders include Alzheimer's disease, asthma, atopic allergy, allergy, atherosclerosis, bronchial asthma, eczema, glomerulonephritis, graft vs. host disease, hemolytic anemias, osteoarthritis, inflammatory bowel disease, sepsis, stroke, transplantation of tissue and organs, vasculitis, diabetic retinopathy and ventilator induced lung injury.

Examples of cancer to be treated herein include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

The cannabinoid cocrystal compositions may be administered by any route appropriate to the condition to be treated, including orally, intravenously, topically, as well as by ophthalmic (eye drops), and transdermal (skin patch) modes.

The cannabinoid cocrystal compositions can be used either alone or in combination with other agents in a therapy. For instance, the cannabinoid cocrystal compositions may be co-administered with at least one additional therapeutic agent. Such combination therapies noted above encompass combined administration (where two or more therapeutic agents are included in the same or separate formulations), and separate administration, in which case, administration of the cannabinoid cocrystal composition can occur prior to, simultaneously, and/or following, administration of the additional therapeutic agent and/or adjuvant.

### V. Formulations

Pharmaceutical formulations comprising the cannabinoid cocrystal compositions as prepared by the methods described herein can be formulated for various routes of administration. The cannabinoid cocrystal compositions having the desired degree of purity is optionally mixed with one or more pharmaceutically acceptable excipients (Remington's Pharmaceutical Sciences (1980) 16th edition, Osol, A. Ed.). The cannabinoid cocrystal compositions can be formulated in accordance with standard pharmaceutical practice as a pharmaceutical composition. In embodiments, a cannabinoid cocrystal composition comprises cannabinol and a pharmaceutically acceptable excipient.

A typical formulation is prepared by mixing the cannabinoid cocrystal composition with excipients, such as carriers and/or diluents. Suitable carriers, diluents and other excipients are well known to those skilled in the art and include materials such as carbohydrates, waxes, water soluble and/or swellable polymers, hydrophilic or hydrophobic materials, gelatin, oils, solvents, water and the like. The particular carrier, diluent or other excipient used will depend upon the means and purpose for which the cannabinoid cocrystal composition is being applied. Solvents are generally selected based on solvents recognized by persons skilled in the art as safe (GRAS) to be administered to a mammal.

In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. Acceptable diluents, carriers, excipients and stabilizers are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEENS®, PLURONICS®, or polyethylene glycol (PEG).

The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the cannabinoid cocrystal composition or aid in the manufacturing of the pharmaceutical product. The formulations may be prepared using conventional dissolution and mixing procedures.

Formulation may be conducted by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but may range from about 3 to about 8. Formulation in an acetate buffer at pH 5 is a suitable embodiment.

The cannabinoid cocrystal formulations can be sterile. In particular, formulations to be used for in vivo administration must be sterile. Such sterilization is readily accomplished by filtration through sterile filtration membranes.

The cannabinoid cocrystal composition ordinarily can be stored as a solid composition, a lyophilized formulation or as an aqueous solution.

The pharmaceutical compositions comprising a cannabinoid cocrystal composition can be formulated, dosed and administered in a fashion, i.e., amounts, concentrations, schedules, course, vehicles and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutic amount” of the compound to be administered will be governed by such considerations, and is the minimum amount necessary to prevent, ameliorate, or treat the coagulation factor mediated disorder. Such amount is preferably below the amount that is toxic to the host or renders the host significantly more susceptible to bleeding.

The cannabinoid cocrystal composition can be formulated into pharmaceutical dosage forms to provide an easily controllable dosage of the drug and to enable patient compliance with the prescribed regimen. The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical compositions may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such 1,3-butanediol. The sterile injectable preparation may also be prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The amount of the cannabinoid cocrystal composition that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

The subject matter described herein is directed to the following embodiments:

1. A solid form comprising a coformer and a cannabinoid, wherein the cannabinoid is a non-solid material.

2. The solid form of embodiment 1, wherein the cannabinoid is an oil.

3. The solid form of embodiment 1, wherein the cannabinoid is selected from the group consisting of cannabinol and tetrahydrocannabinol.

4. The solid form of any one of embodiments 1-3, wherein the coformer is selected from the group consisting of potassium, calcium, carnitine, aspartame, L-proline, D-proline, L-Arginine, L-Lysine, betaine, tetramethylpyrazine, 1H-Imidazole, nicotinic acid, saccharin, urea, and nicotinamide.

5. The solid form of any one of embodiments 1-4, wherein the cannabinoid is cannabinol and the coformer is tetramethylpyrazine.

6. The solid form of embodiment 5, wherein the molar ratio of cannabinol to tetramethylpyrazine is about 1 to 0.5.

7. The solid form of embodiment 6, characterized by a powder X-ray diffraction pattern comprising at least one peak selected from the group consisting of 3.92, 7.89, 8.98, 10.70, 10.94, 11.42, 12.70, 13.42, 13.99, 14.55, 14.81, 15.49, 15.86, 16.14, 16.78, 17.18, 17.41, 17.66, 18.10, 18.71, 20.08, 20.47, 21.05, 21.22, 21.70, 22.23, 22.75, 23.28, 23.91, 24.81, 25.58, 27.04, 27.45, 28.57, 29.10, 29.88, and 30.62 (each degrees 2θ±0.20).

8. The solid form of embodiment 7, characterized by a powder X-ray diffraction pattern comprising at least two peaks selected from the group consisting of 3.92, 7.89, 8.98, 10.70, 10.94, 11.42, 12.70, 13.42, 13.99, 14.55, 14.81, 15.49, 15.86, 16.14, 16.78, 17.18, 17.41, 17.66, 18.10, 18.71, 20.08, 20.47, 21.05, 21.22, 21.70, 22.23, 22.75, 23.28, 23.91, 24.81, 25.58, 27.04, 27.45, 28.57, 29.10, 29.88, and 30.62 (each degrees 2θ±0.20).

9. The solid form of embodiment 8, characterized by a powder X-ray diffraction pattern comprising at least three peaks selected from the group consisting of 3.92, 7.89, 8.98, 10.70, 10.94, 11.42, 12.70, 13.42, 13.99, 14.55, 14.81, 15.49, 15.86, 16.14, 16.78, 17.18, 17.41, 17.66, 18.10, 18.71, 20.08, 20.47, 21.05, 21.22, 21.70, 22.23, 22.75, 23.28, 23.91, 24.81, 25.58, 27.04, 27.45, 28.57, 29.10, 29.88, and 30.62 (each degrees 2θ±0.20).

10. The solid form of embodiment 6, characterized by a powder X-ray diffraction pattern substantially as depicted in FIG. 6.

11. The solid form of embodiment 6, characterized by a DSC thermogram with a peak onset at about 72.8° C. and a peak maximum at about 74.3° C.

12. The solid form of embodiment 6, characterized by a DSC thermogram substantially as depicted in FIG. 7.

13. The solid form of embodiment 6, characterized by a Raman spectrum comprising at least one peak selected from the group consisting of 169, 228, 320, 365, 379, 408, 477, 509, 536, 549, 564, 581, 608, 664, 693, 714, 732, 774, 863, 883, 998, 1028, 1107, 1156, 1192, 1241, 1284, 1297, 1339, 1375, 1437, 1497, 1513, 1570, 1582, 1617, 2850, 2924, 2979, and 3039 (each cm⁻¹).

14. The solid form of embodiment 6, characterized by a Raman spectrum substantially as depicted in FIG. 5.

15. The solid form of embodiment 6, wherein the solid form is crystalline.

16. The solid form of embodiment 6, wherein the solid form is a cocrystal.

17. The solid form of embodiment 6, wherein the solid form is substantially free of solvent.

18. The solid form of embodiment 6, wherein the solid form is stable.

19. The solid form of embodiment 5, wherein the molar ratio of cannabinol to tetramethylpyrazine is about 1 to 1.

20. The solid form of embodiment 19, characterized by a powder X-ray diffraction pattern comprising at least one peak selected from the group consisting of 6.91, 8.75, 10.29, 10.56, 11.13, 12.36, 13.76, 14.77, 15.24, 15.72, 16.41, 17.78, 18.80, 20.07, 20.48, 20.91, 21.76, 22.69, 23.24, 23.46, 23.95, 24.46, 24.86, 25.70, 27.32, 27.94, 28.45, 28.90, 31.45, 31.95, 35.57, and 36.71 (each degrees 2θ±0.20).

21. The solid form of embodiment 20, characterized by a powder X-ray diffraction pattern comprising at least two peaks selected from the group consisting of 6.91, 8.75, 10.29, 10.56, 11.13, 12.36, 13.76, 14.77, 15.24, 15.72, 16.41, 17.78, 18.80, 20.07, 20.48, 20.91, 21.76, 22.69, 23.24, 23.46, 23.95, 24.46, 24.86, 25.70, 27.32, 27.94, 28.45, 28.90, 31.45, 31.95, 35.57, and 36.71 (each degrees 2θ±0.20).

22. The solid form of embodiment 21, characterized by a powder X-ray diffraction pattern comprising at least three peaks selected from the group consisting of 6.91, 8.75, 10.29, 10.56, 11.13, 12.36, 13.76, 14.77, 15.24, 15.72, 16.41, 17.78, 18.80, 20.07, 20.48, 20.91, 21.76, 22.69, 23.24, 23.46, 23.95, 24.46, 24.86, 25.70, 27.32, 27.94, 28.45, 28.90, 31.45, 31.95, 35.57, and 36.71 (each degrees 2θ±0.20).

23. The solid form of embodiment 19, characterized by a powder X-ray diffraction pattern substantially as depicted in FIG. 10.

24. The solid form of embodiment 19, characterized by a DSC thermogram with a peak onset at about 76.3° C. and a peak maximum at about 77.8° C.

25. The solid form of embodiment 19, characterized by a DSC thermogram substantially as depicted in FIG. 11.

26. The solid form of embodiment 19, characterized by a Raman spectrum comprising at least one peak selected from the group consisting of 166, 229, 245, 264, 322, 343, 379, 409, 479, 507, 535, 583, 598, 626, 667, 703, 724, 862, 1027, 1101, 1158, 1191, 1225, 1240, 1284, 1300, 1342, 1385, 1440, 1506, 1549, 1573, 1584, 1607, 1618, 2856, 2925, 2969, 2987, and 3036 (each cm⁻¹).

27. The solid form of embodiment 19, characterized by a Raman spectrum substantially as depicted in FIG. 9.

28. The solid form of embodiment 19, wherein the solid form is crystalline.

29. The solid form of embodiment 19, wherein the solid form is a cocrystal.

30. The solid form of embodiment 19, wherein the solid form is substantially free of solvent.

31. The solid form of embodiment 19, wherein the solid form is stable.

32. The solid form of embodiment 1, wherein the cannabinoid is cannabinol and the coformer is L-proline.

33. The solid form of embodiment 32, wherein the molar ratio of cannabinol to L-proline is about 1 to 1.

34. The solid form of embodiment 33, characterized by a powder X-ray diffraction pattern comprising at least one peak selected from the group consisting of 4.72, 6.19, 8.21, 9.47, 10.98, 12.56, 13.75, 14.25, 16.03, 16.48, 17.15, 19.04, 19.85, 20.77, 21.50, 21.84, 23.09, 23.90, 24.80, 25.91, 26.62, 27.20, 37.69, and 38.26 (each degrees 2θ±0.20).

35. The solid form of embodiment 34, characterized by a powder X-ray diffraction pattern comprising at least two peaks selected from the group consisting of 4.72, 6.19, 8.21, 9.47, 10.98, 12.56, 13.75, 14.25, 16.03, 16.48, 17.15, 19.04, 19.85, 20.77, 21.50, 21.84, 23.09, 23.90, 24.80, 25.91, 26.62, 27.20, 37.69, and 38.26 (each degrees 2θ±0.20).

36. The solid form of embodiment 35, characterized by a powder X-ray diffraction pattern comprising at least three peaks selected from the group consisting of 4.72, 6.19, 8.21, 9.47, 10.98, 12.56, 13.75, 14.25, 16.03, 16.48, 17.15, 19.04, 19.85, 20.77, 21.50, 21.84, 23.09, 23.90, 24.80, 25.91, 26.62, 27.20, 37.69, and 38.26 (each degrees 2θ±0.20).

37. The solid form of embodiment 33, characterized by a powder X-ray diffraction pattern substantially as depicted in FIG. 15.

38. The solid form of embodiment 33, characterized by a DSC thermogram with a peak onset at about 120.7° C. and a peak maximum at about 124.3° C.

39. The solid form of embodiment 33, characterized by a DSC thermogram substantially as depicted in FIG. 16.

40. The solid form of embodiment 33, characterized by a Raman spectrum comprising at least one peak selected from the group consisting of 163, 240, 324, 362, 408, 486, 509, 530, 551, 580, 621, 671, 696, 711, 773, 841, 863, 915, 935, 995, 1027, 1110, 1157, 1194, 1236, 1297, 1330, 1400, 1444, 1496, 1509, 1583, 1615, 2885, 2923, 2972, and 3020 (each cm⁻¹).

41. The solid form of embodiment 33, characterized by a Raman spectrum substantially as depicted in FIG. 14.

42. The solid form of embodiment 33, wherein the solid form is crystalline.

43. The solid form of embodiment 33, wherein the solid form is a cocrystal.

44. The solid form of embodiment 33, wherein the solid form is substantially free of solvent.

45. The solid form of embodiment 33, wherein the solid form is stable.

46. The solid form of embodiment 1, wherein the cannabinoid is cannabinol and the coformer is D-proline.

47. The solid form of embodiment 46, wherein the molar ratio of cannabinol to D-proline is about 1 to 1.

48. The solid form of embodiment 47, characterized by a powder X-ray diffraction pattern comprising at least one peak selected from the group consisting of 4.70, 8.18, 9.44, 10.95, 12.53, 15.99, 16.44, 17.11, 19.01, 19.82, 20.74, 21.46, 21.81, 23.01, 23.93, 24.77, 26.55, 27.14, 37.64, and 38.24 (each degrees 2θ±0.20).

49. The solid form of embodiment 48, characterized by a powder X-ray diffraction pattern comprising at least two peaks selected from the group consisting of 4.70, 8.18, 9.44, 10.95, 12.53, 15.99, 16.44, 17.11, 19.01, 19.82, 20.74, 21.46, 21.81, 23.01, 23.93, 24.77, 26.55, 27.14, 37.64, and 38.24 (each degrees 2θ±0.20).

50. The solid form of embodiment 49, characterized by a powder X-ray diffraction pattern comprising at least three peaks selected from the group consisting of 4.70, 8.18, 9.44, 10.95, 12.53, 15.99, 16.44, 17.11, 19.01, 19.82, 20.74, 21.46, 21.81, 23.01, 23.93, 24.77, 26.55, 27.14, 37.64, and 38.24 (each degrees 2θ±0.20).

51. The solid form of embodiment 47, characterized by a powder X-ray diffraction pattern substantially as depicted in FIG. 29.

52. The solid form of embodiment 47, characterized by a DSC thermogram with a peak onset at about 119.5° C. and a peak maximum at about 125.3° C.

53. The solid form of embodiment 47, characterized by a DSC thermogram substantially as depicted in FIG. 30.

54. The solid form of embodiment 47, characterized by a Raman spectrum comprising at least one peak selected from the group consisting of 163, 239, 323, 362, 408, 486, 508, 531, 551, 580, 621, 671, 696, 711, 773, 841, 863, 915, 995, 1027, 1110, 1157, 1193, 1236, 1298, 1329, 1399, 1443, 1508, 1583, 1615, 2924, 2985, and 3021 (each cm⁻¹).

55. The solid form of embodiment 47, characterized by a Raman spectrum substantially as depicted in FIG. 28.

56. The solid form of embodiment 47, wherein the solid form is crystalline.

57. The solid form of embodiment 47, wherein the solid form is a cocrystal.

58. The solid form of embodiment 47, wherein the solid form is substantially free of solvent.

59. The solid form of embodiment 47, wherein the solid form is stable.

60. A pharmaceutical composition comprising the solid form of any one of embodiments 1-59.

61. The pharmaceutical composition of embodiment 60, further comprising a pharmaceutically acceptable excipient.

62. A method of preparing a solid form comprising a coformer and a cannabinoid, starting from a cannabinoid oil selected from the group consisting of cannabinol and tetrahydrocannabinol, said method comprising:
contacting said cannabinoid with said coformer and a solvent to prepare a suspension;
seeding said suspension with a seed cocrystal;
heating said suspension at a first temperature with stirring; and,
separating a solid material from said suspension, wherein said solid material is a cocrystal comprising said cannabinoid and said coformer.

63. The method of embodiment 62, wherein said cannabinoid is cannabinol.

64. The method of embodiment 62, wherein said coformer is selected from the group consisting of potassium, calcium, carnitine, aspartame, L-proline, D-proline, L-Arginine, L-Lysine, betaine, tetramethylpyrazine, 1H-Imidazole, nicotinic acid, saccharin, urea, and nicotinamide.

65. The method of embodiment 64, wherein said coformer is selected from the group consisting of tetramethylpyrazine, L-proline, and D-proline.

66. The method of embodiment 62, wherein said solvent is selected from the group consisting of isooctane, petroleum ether, methanol, isopropanol, acetonitrile, acetone, tetrahydrofuran, 1,4-dioxane, ethyl acetate, 4-methyl-2-pentanone, dichloromethane, methyl t-butyl ether, and toluene.

67. The method of embodiment 66, wherein said solvent is isooctane or petroleum ether.

68. The method of embodiment 62, wherein said solid form is crystalline.

69. The method of embodiment 62, wherein said solid form is a cocrystal.

70. The method of embodiment 62, wherein said cocrystal is characterized by a powder X-ray diffraction pattern substantially as depicted in any one of FIG. 6, 10, 15, 20, 25, 29, 34, or 39.

The disclosed subject matter is further described in the following non-limiting Examples. It should be understood that these Examples, while indicating preferred embodiments of the invention, are given by way of illustration only.

### Examples

### Materials and Methods

### FT-Raman Spectroscopy

Raman spectra were collected with a Nicolet NXR9650 or NXR 960 spectrometer (Thermo Electron) equipped with 1064 nm Nd:YVO4 excitation laser, InGaAs and liquid-N2 cooled Ge detectors, and a MicroStage. All spectra were acquired at 4 cm⁻¹ resolution, 64-256 scans, using Happ-Genzel apodization function and 2-level zero-filling.

### Polarized-Light Microscopy (PLM)

The photomicrographs were collected using an Olympus BX60 polarized-light microscope equipped with an Olympus DP70 camera.

### Powder X-Ray Diffraction (PXRD) PANalytical

PXRD diffractograms were acquired on a PANalytical X'Pert Pro diffractometer using Ni-filtered Cu Ka (45 kV/40 mA) radiation and a step size of 0.03° 2θ and X'celerator™ RTMS (Real Time Multi-Strip) detector. Configuration on the incidental beam side: variable divergence slits (10 mm irradiated length), 0.04 rad Soller slits, fixed anti-scatter slit (0.50°), and 10 mm beam mask. Configuration on the diffracted beam side: variable anti-scatter slit (10 mm observed length) and 0.04 rad Soller slit. Samples were mounted flat on zero-background Si wafers.

### Powder X-Ray Diffraction (PXRD) Bruker

PXRD diffractograms were acquired on a Bruker D8 Advance system (SN:2631) using Cu Ka (40 kV/40 mA) radiation and a step size of 0.03° 2θ and LynxEye detector. Configuration on the incident beam side: Goebel mirror, mirror exit slit (0.2 mm), 2.5 deg Soller slits, beam knife. Configuration on the diffracted beam side: anti-scatter slit (8 mm) and 2.5 deg. Soller slit. Samples were mounted flat on zero-background Si wafers.

Differential Scanning calorimetry (DSC)

DSC was conducted with a TA Instruments Q100 or Q2000 differential scanning calorimeter equipped with an autosampler and a refrigerated cooling system under 40 mL/min N2 purge. DSC thermograms of samples were obtained at 15° C./min in crimped Al pans, unless noted otherwise.

### Thermogravimetric Analysis (TGA)

TGA thermograms were obtained with a TA Instruments Q50 thermogravimetric analyzer under 40 mL/min N2 purge in Pt or Al pans. TGA thermograms of samples were obtained at 15° C./min, unless noted otherwise.

Thermogravimetric Analysis with IR Off-Gas Detection (TGA-IR)

TGA-IR was conducted with a TA Instruments Q5000 thermogravimetric analyzer interfaced to a Nicolet 6700 FT-IR spectrometer (Thermo Electron) equipped with an external TGA-IR module with a gas flow cell and DTGS detector. TGA was conducted with 25 mL/min N2 flow and heating rate of 15° C./min in Pt or Al pans. IR spectra were collected at 4 cm⁻¹ resolution and 32 scans at each time point.

### Proton Nuclear Magnetic Resonance (¹H NMR).

The ¹H NMR spectra were collected using Agilent DD2 500 MHz spectrometer with TMS reference. Samples were dissolved in DMSO-d6 or CD₃OD.

### Characterization of Cannabinol Starting Material

Cannabinol can be obtained as an oily extract from the cannabis plant. Synthetic cannabinol can also be prepared in the laboratory. The cannabinol used in the cocrystal investigations described herein was prepared as follows:
1. D9-THC-Naphthoylester was reacted in a vessel with 2.2 molar equivalents of sulfur and heated to about 250° C. without solvent under a gentle nitrogen purge. After 60-90 minutes, the vessel was cooled to 60° C. to reveal a dark-brown mixture, CBN-Naphthoylester.
2. The CBN-Naphthoylester obtained in 1 was saponified in THF/MeOH/Water with Lithium-hydroxide. The reaction mixture was stirred for about 1 hour at 37-42° C. T-butyl methyl ether was added to the reaction mixture. The organic layer was washed with a solution of sodium carbonate and sodium ascorbate in water. The organic layer was washed by sodium carbonate in water. Activated Carbon was added to the organic layer. The activated carbon/reaction mixture slurry was heated to about 60° C. and stirred for about 1 hour. The mixture was cooled to room temperature and filtered.
3. The wet cake was washed with t-butyl methyl ether to remove more product from the carbon cake. The solvents were removed via distillation to afford an oily crude product.
4. At a warmer temperature (e.g. at 60-70° C.), the crude CBN oil was poured and blended into silica gel to form a homogenous mixture, which was subjected to a fresh silica gel pad pre-loaded in a column. The product was flashed out using a mixture of n-heptane and ethyl acetate as the eluent. Fractions were combined based on HPLC analysis. The solvents were removed to afford a light brown crude oil. The crude oily product was transferred to a 3-L 1-neck round bottom flask and distilled to further remove the solvents (A final temperature of 100-150° C. is reached with a diaphragm vacuum pump).
5. After the removal of all the solvents, the distillation was continued with a fractional distillation head and a high vacuum oil pump (e.g. 0-1.0 mbar). The first fraction (˜15%) was collected at a temperature between 210-219° C. The second fraction (˜70%), a light yellow-clear oily product, was collected at a temperature between 226-230° C. The final fraction (˜15%) was collected at 245° C. or higher. The fractions were tested by HPLC.

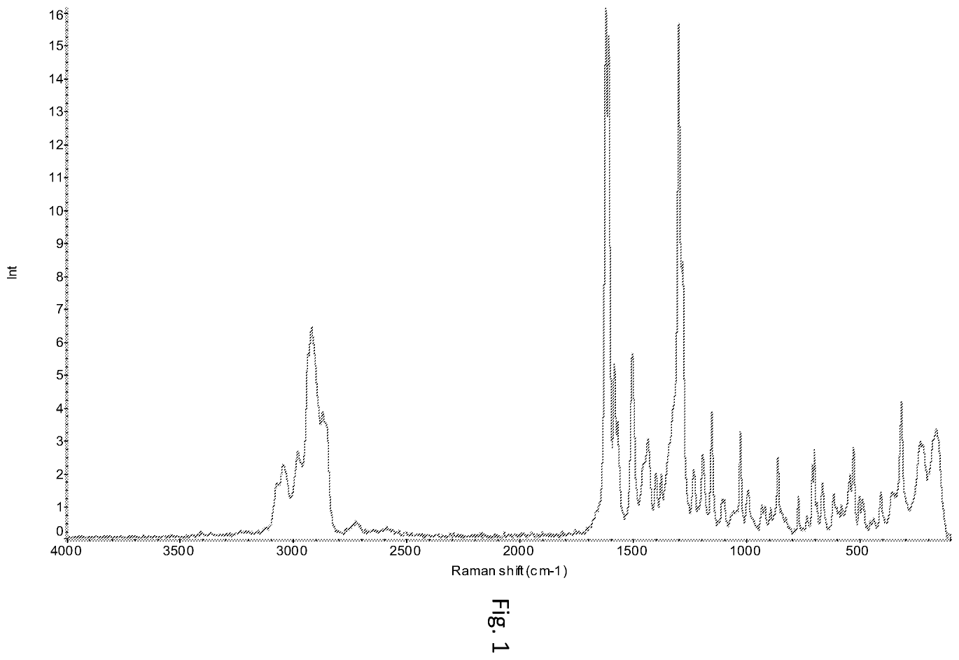

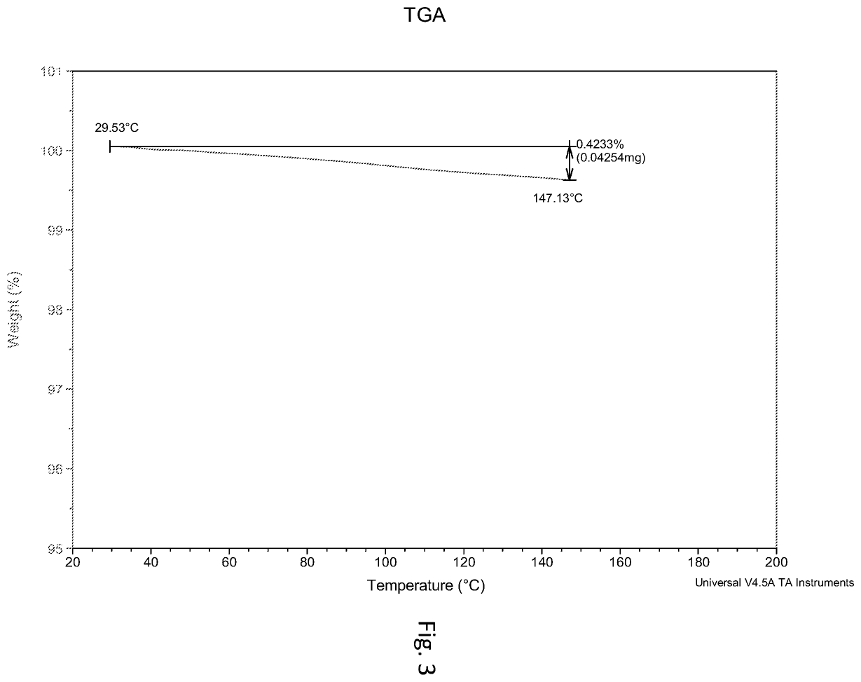

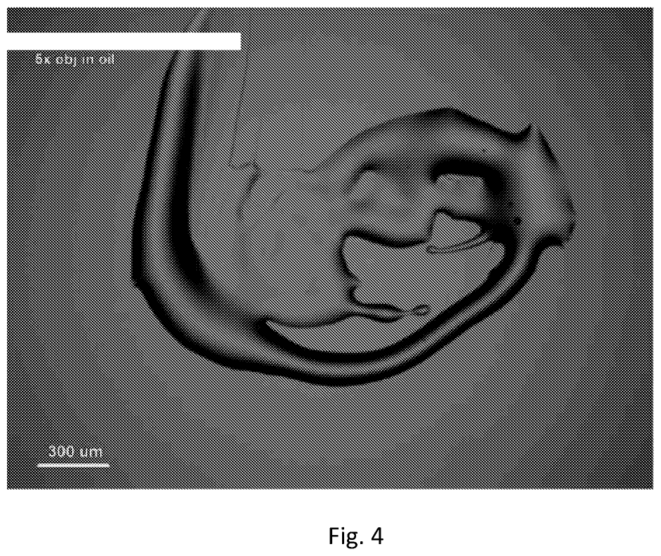

The cannabinol starting material (API) was determined to be an amorphous, viscous oil by visual and PLM examination (FIG. 4). TGA-IR showed 0.4% weight loss upon heating to 147° C. (FIG. 3). The solubility of the material in various solvents was also assessed (Table 1). The cannabinol was determined to be highly soluble/miscible (>100 mg/mL) in common organic solvents and poorly soluble/miscible (<20 mg/mL) in water (denatured with 1% MeOH). FIG. 1 shows its FT-Raman spectrum, characterized by peaks at 164, 235, 318, 409, 505, 530, 546, 617, 668, 702, 711, 774, 863, 994, 1029, 1155, 1195, 1234, 1301, 1378, 1401, 1438, 1506, 1585, 1611, 1622, 2921, and 3048 (each cm⁻¹).

TABLE 1

| | | Solubility | Solubility at |
|---|---|---|---|
| | | at RT | 40° C. |
| # | Solvent | [mg/mL] | [mg/mL] |
| 1 | Methanol | >100 | n/a |
| 2 | Isopropanol | >100 | n/a |
| 3 | Acetonitrile | >100 | n/a |
| 4 | Acetone | >100 | n/a |
| 5 | Tetrahydrofuran | >100 | n/a |
| 6 | 1,4-Dioxane | >100 | n/a |
| 7 | Ethyl acetate | >100 | n/a |
| 8 | 4-Methyl-2-pentanone | >100 | n/a |
| 9 | Dichloromethane | >100 | n/a |
| 10 | Methyl t-butyl ether | >100 | n/a |
| 11 | Toluene | >100 | n/a |
| 12 | Water (1% Methanol) | <20 | <20 |

n/a - not applicable

### Example 1: Solid Form Screening Experiments

Fifteen (15) cocrystal formers (CCFs) were examined in the solid form screening experiments. Either one or four equivalents of CCF relative to the API were dosed in the screens. Table 2 presents the set of CCFs utilized and the equivalents dosed.

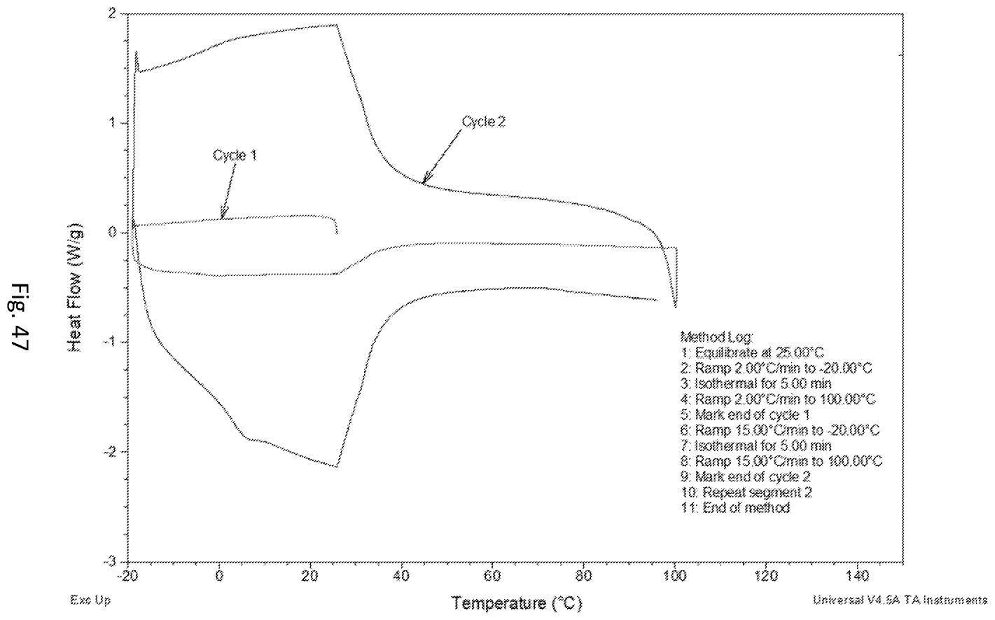

Solid form screening experiments involving just the parent API in several solvents were unsuccessful in producing crystalline hits and instead yielded amorphous materials or viscous solutions. In addition to the various modes employed for co-crystallization, DSC cycling was conducted on the cannabinol starting material. The trace (shown in FIG. 47) showed no evidence of crystallization.

TABLE 2

| # | Cocrystal Formers | Eq. |
|---|---|---|
| 1 | Potassium | 1 |
| 2 | Calcium | 1 |
| 3 | Carnitine | 1 |
| 4 | Aspartame | 1 |
| 5 | L-proline | 1 |
| 6 | D-proline | 1 |
| 7 | L-Arginine | 1 |
| 8 | L-Lysine | 1 |
| 9 | Betaine | 1 |
| 10 | Tetramethylpyrazine | 4 |
| 11 | 1H-Imidazole | 4 |
| 12 | Nicotinic acid | 4 |
| 13 | Saccharin | 4 |
| 14 | Urea | 4 |
| 15 | Nicotinamide | 4 |

Seven neat solvents and one solvent mixture were utilized in the cocrystal screening experiments: acetonitrile, 1,4-dioxane, isobutyl acetate, heptane, cyclohexane, isooctane, petroleum ether, and isopropanol/water (9:1 v/v).

A total of 139 screening experiments were conducted utilizing several crystallization modes and temperatures ranging from 5° C. to 40° C. The crystallization methods included stirring and temperature-cycling (TC), rapid cooling (RC), evaporation of solutions (EV), solvent-drop grinding (SDG), sonication, and seeding.

The solid form screening experiments were undertaken as follows:
1. Dissolve API starting material in MeOH at 100 mg/mL concentration.
2. Dispense 200 μL of API/MeOH solution into screening vials.
3. Rapidly evaporate MeOH under reduced pressure (GeneVac®).
4. Combine 50-300 μL of reaction solvent with the amorphous API (20 mg).
5. To the resulting solutions, add 1.0 or 4.0 equivalents of CCFs at RT.
6. Stir the samples while cycling the temperature between 40° C. and 5° C. (1 hour at each temperature) for three days (TC1). Isolate birefringent solids under nitrogen.
7. Evaporate solvents under reduced pressure using GeneVac,® and re-dispense 50-100 μL of the original solvents.
8. Scratch vials inside with probe, and stir the samples while cycling the temperature between 40° C. and 5° C. for up to two weeks (TC2). Isolate birefringent solids under nitrogen.
9. Cool solutions from step 8 to 5° C. and hold for 24 hours.
10. Scratch vials inside with probe, and evaporate solvents in nitrogen purge chamber for 14 days. Isolate birefringent solids.
11. Solvent-swap two reaction solvents per CCF (select non-birefringent samples) for isooctane and petroleum ether. Scratch vials inside with probe, and stir the samples while cycling the temperature between 40° C. and 5° C. (1 hour at each temperature) for two days. Isolate birefringent solids under nitrogen.
12. Add back minimal amount of reaction solvent if needed (e.g. for evaporated solutions). Sonicate samples for six intervals of 30 minutes each. (SN). Isolate birefringent solids under nitrogen.
13. Seed suspensions/solutions/gums/oils with D-Proline screening hit (see Example 11 for detailed synthesis), scratch vials inside with probe, and stir while cycling the temperature between 25° C. and 5° C. (1 hour at each temperature) for seven days (HS1). Isolate birefringent solids under nitrogen.
14. Seed suspensions/solutions/gums/oils with Cannabidiol, scratch vials inside with probe, and stir while cycling the temperature between 40° C. and 5° C. (1 hour at each temperature) for six days (HS2). Isolate birefringent solids under nitrogen.

All outputs of the screen were assessed for crystallinity by visual and/or PLM analysis. Birefringent solids were analyzed by PXRD and compared to the PXRD patterns of the API and CCFs to assess cocrystal formation. For potential cocrystal hits, additional analyses were conducted [e.g. FT-Raman, DSC, and TGA-IR as sample quantity permitted]. Two different crystal forms—designated as Group A (Form A) and Group B (Form B)—were discovered during the screening experiments. In later scale-up experiments, a third Group C (Form C), was isolated. The cocrystals obtained in each group shared similar PXRD patterns with one another.

Experiments led to isolatable crystalline hits from four CCFs: Tetramethylpyrazine (0.5 and 1 eq cocrystals), L-Proline (1 eq cocrystals), D-Proline (1 eq cocrystals), and Nicotinamide (mixture with CCF). The other CCFs produced birefringent materials which deliquesced upon isolation, crystalline CCF, CCF/API mixtures, or amorphous materials. Table 3 shows the products of the original screening experiments (six solvents). Crystal Form A was the predominant form observed in the original set of screens.

TABLE 3

| No. | Coformer | IPA/water (9:1) | Acetonitrile | 1,4-Dioxane |
|---|---|---|---|---|
| 1 | Potassium | ∧ ∧ ∧ ∧ ∧ ∧ ∧ ∧ ∧ ∧ ∧ ∧ | ∧ ∧ ∧ ∧ ∧ ∧ ∧ ∧ ∧ ∧ ∧ ∧ | ------------------ |
| 2 | Calcium | ******* \\\\\\\\\\ | ∧ ∧ ∧ ∧ ∧ ∧ ∧ ∧ ∧ ∧ ∧ ∧ | ******* \\\\\\\\\\ |
| 3 | Camitine | ------------------ | ∧ ∧ ∧ ∧ ∧ ∧ ∧ ∧ ∧ ∧ ∧ ∧ | ∧ ∧ ∧ ∧ ∧ ∧ ∧ ∧ ∧ ∧ ∧ ∧ |
| 4 | Aspartame | ******* \\\\\\\\\\ | ******* \\\\\\\\\\ | ******* \\\\\\\\\\ |
| 5 | L-Proline | A,1++++ \\\\\\\\\\ | A,1+++++++++++ | ******* \\\\\\\\\\ |
| 7 | L-Arginine | ******* \\\\\\\\\\ | ******* \\\\\\\\\\ | ∧ ∧ ∧ ∧ ∧ ∧ ∧ ∧ ∧ ∧ ∧ ∧ |
| 8 | L-Lysine | ∧ ∧ ∧ ∧ ∧ ∧ ∧ ∧ ∧ ∧ ∧ ∧ | 2\\\\\\\\\\\\\\\\\ | 2\\\\\\\\\\\\\\\\\ |
| 9 | Betaine | ------------------ | ------------------ | ∧ ∧ ∧ ∧ ∧ ∧ ∧ ∧ ∧ ∧ ∧ ∧ |
| 10 | Tetramethylpyrazine | ∧ ∧ ∧ ∧ ∧ ∧ ∧ ∧ ∧ ∧ ∧ ∧ | ∧ ∧ ∧ ∧ ∧ ∧ ∧ ∧ ∧ ∧ ∧ ∧ | ∧ ∧ ∧ ∧ ∧ ∧ ∧ ∧ ∧ ∧ ∧ ∧ |
| 11 | 1H-imidazole | ------------------ | ------------------ | ------------------ |
| 12 | Nicotinic acid | ******* \\\\\\\\\\ | ******* \\\\\\\\\\ | ******* \\\\\\\\\\ |
| 13 | Saccharin | ******* \\\\\\\\\\ | ******* \\\\\\\\\\ | ******* \\\\\\\\\\ |
| 14 | Urea | ------------------ | ******* \\\\\\\\\\ | ******* \\\\\\\\\\ |
| 15 | Nicotinamide | 2\\\\\\\\\\\\\\\\\ | \\\\\\\\\\\\\\\\\\ | 2\\\\\\\\\\\\\\\\\ |
| 16 | Parent | ∧ ∧ ∧ ∧ ∧ ∧ ∧ ∧ ∧ ∧ ∧ ∧ | ∧ ∧ ∧ ∧ ∧ ∧ ∧ ∧ ∧ ∧ ∧ ∧ | ∧ ∧ ∧ ∧ ∧ ∧ ∧ ∧ ∧ ∧ ∧ ∧ |
| No. | Coformer | Isobutyl Acetate | Heptane | Cyclohexane |
| 1 | Potassium | ∧ ∧ ∧ ∧ ∧ ∧ ∧ ∧ ∧ ∧ ∧ ∧ | ∧ ∧ ∧ ∧ ∧ ∧ ∧ ∧ ∧ ∧ ∧ ∧ | ∧ ∧ ∧ ∧ ∧ ∧ ∧ ∧ ∧ ∧ ∧ ∧ |
| 2 | Calcium | ******* \\\\\\\\\\ | ******* \\\\\\\\\\ | ∧ ∧ ∧ ∧ ∧ ∧ ∧ ∧ ∧ ∧ ∧ ∧ |
| 3 | Camitine | ------------------ | ------------------ | ------------------ |
| 4 | Aspartame | ******* \\\\\\\\\\ | ∧ ∧ ∧ ∧ ∧ ∧ ∧ ∧ ∧ ∧ ∧ ∧ | ?????????? |
| 5 | L-Proline | ∧ ∧ ∧ ∧ ∧ ∧ ∧ ∧ ∧ ∧ ∧ ∧ | ∧ ∧ ∧ ∧ ∧ ∧ ∧ ∧ ∧ ∧ ∧ ∧ | ?????????? |
| 7 | L-Arginine | ******* \\\\\\\\\\ | ******* \\\\\\\\\\ | ******* |
| 8 | L-Lysine | ∧ ∧ ∧ ∧ ∧ ∧ ∧ ∧ ∧ ∧ ∧ ∧ | 2\\\\\\\\\\\\\\\\\ | 2\\\\\\\\\\\\\\\\\ |
| 9 | Betaine | ∧ ∧ ∧ ∧ ∧ ∧ ∧ ∧ ∧ ∧ ∧ ∧ | ------------------ | ------------------ |
| 10 | Tetramethylpyrazine | ∧ ∧ ∧ ∧ ∧ ∧ ∧ ∧ ∧ ∧ ∧ ∧ | A\|\|\|\|\|\|\|\|\|\|\|\|\|\|\| | ∧ ∧ ∧ ∧ ∧ ∧ ∧ ∧ ∧ ∧ ∧ ∧ |
| 11 | 1H-imidazole | ------------------ | | ∧ ∧ ∧ ∧ ∧ ∧ ∧ ∧ ∧ ∧ ∧ ∧ |
| 12 | Nicotinic acid | ******* \\\\\\\\\\ | ******* \\\\\\\\\\ | ******* \\\\\\\\\\ |
| 13 | Saccharin | ******* \\\\\\\\\\ | ******* \\\\\\\\\\ | ******* \\\\\\\\\\ |
| 14 | Urea | ∧ ∧ ∧ ∧ ∧ ∧ ∧ ∧ ∧ ∧ ∧ ∧ | ******* \\\\\\\\\\ | ∧ ∧ ∧ ∧ ∧ ∧ ∧ ∧ ∧ ∧ ∧ ∧ |
| 15 | Nicotinamide | 2\\\\\\\\\\\\\\\\\ | A,3\|\|\|\|\|\| \\\\\\\\\\ | \\\\\\\\\\\\\\\\\\ |
| 16 | Parent | ∧ ∧ ∧ ∧ ∧ ∧ ∧ ∧ ∧ ∧ ∧ ∧ | ∧ ∧ ∧ ∧ ∧ ∧ ∧ ∧ ∧ ∧ ∧ ∧ | ∧ ∧ ∧ ∧ ∧ ∧ ∧ ∧ ∧ ∧ ∧ ∧ |

Notes:
A, B - crystal forms
1 - Deliquesced at ambient w/in 3 days (lab humidity 60%)
2 - Based on PXRD (FT-Raman analysis not peformed)
3 - Analysis of 19-day old suspension showed all coformer
|||||||| Birefringent
+++++++ Poorly birefringent
******* Parent
\\\\\\\\\ Coformer
---------- Deliquescence
∧∧∧∧∧∧∧ Amorphous (Mostly)

In addition to the screening experiments in the original six solvents, two additional solvents (isooctane and petroleum ether) were investigated by swapping two reaction solvents per CCF for isooctane and petroleum ether. Samples exhibiting the least birefringence were selected for this substitution. Table 4 shows the products of solid form screening in the two new solvents. Both crystal forms A and B were observed in these screens. Several detailed syntheses of these products (used as seeds in further examples) are provided in Examples 8-10.

TABLE 4

| No. | Coformer | Petroleum Ether | Isooctane |
|---|---|---|---|
| 1 | Potassium | ∧∧∧∧∧∧ | ∧∧∧∧∧∧ |
| | | ∧∧∧∧∧∧ | ∧∧∧∧∧∧ |
| 2 | Calcium | ∧∧∧∧∧∧ | ∧∧∧∧∧∧ |
| | | ∧∧∧∧∧∧ | ∧∧∧∧∧∧ |
| 3 | Camitine | ∧∧∧∧∧∧ | ∧∧∧∧∧∧ |
| | | ∧∧∧∧∧∧ | ∧∧∧∧∧∧ |
| 4 | Aspartame | ∧∧∧∧∧∧ | ∧∧∧∧∧∧ |
| | | ∧∧∧∧∧∧ | ∧∧∧∧∧∧ |
| 5 | L-Proline | B,2\|\|\|\|\|\|\|\|\|\|\|\|\|\|\| | B,2\|\|\|\|\|\|\|\|\|\|\|\|\|\|\| |
| 6 | D-Proline | B,2\|\|\|\|\|\|\|\|\|\|\|\|\|\|\| | B,2\|\|\|\|\|\|\|\|\|\|\|\|\|\|\| |
| 7 | L-Arginine | ∧∧∧∧∧∧ | ∧∧∧∧∧∧ |
| | | ∧∧∧∧∧∧ | ∧∧∧∧∧∧ |
| 8 | L-Lysine | ∧∧∧∧∧∧ | ∧∧∧∧∧∧ |
| | | ∧∧∧∧∧∧ | ∧∧∧∧∧∧ |
| 9 | Betaine | ∧∧∧∧∧∧ | ∧∧∧∧∧∧ |
| | | ∧∧∧∧∧∧ | ∧∧∧∧∧∧ |
| 10 | Tetramethylpyrazine | 3 | A + B\|\|\|\|\|\|\|\|\|\|\|\|\|\|\| |
| 11 | 1H-imidazole | ∧∧∧∧∧∧ | ∧∧∧∧∧∧ |
| | | ∧∧∧∧∧∧ | ∧∧∧∧∧∧ |
| 12 | Nicotinic acid | 1\\\\\\\\\\\\\\\\\\ | 1\\\\\\\\\\\\\\\\\\ |
| 13 | Saccharin | 1\\\\\\\\\\\\\\\\\\ | ******* \\\\\\\\\\ |
| 14 | Urea | 1\\\\\\\\\\\\\\\\\\ | 1\\\\\\\\\\\\\\\\\\ |
| 15 | Nicotinamide | 1\\\\\\\\\\\\\\\\\\ | 1\\\\\\\\\\\\\\\\\\ |
| 16 | Parent | 3 | 3 |

Notes:
A, B - crystal forms
1 - Based on PXRD (FT-Ramen analysis is not peformed)
2 - Coformer peaks increase over time at ambient
3 - Viscous solution
|||||||| Birefringent
******* Parent
\\\\\\\\\ Coformer
∧∧∧∧∧∧∧ No solid

### Example 2: Solvent-Drop Grinding Experiments

Solvent-drop grinding experiments with MeOH were conducted for several CCFs. However, these experiments did not yield any cocrystal hits.

### Example 3: Cocrystal Hits of Cannabinol

Non-solvated Tetramethylpyrazine Group A and Group B cocrystals, and non-solvated L-Proline Group C and D-Proline Group C cocrystals were obtained. L-Proline Group A and D-Proline Group A cocrystals were poorly crystalline, and they deliquesced at ambient conditions within three days. L-Proline Group B and D-Proline Group B cocrystals were found to be classes of isostructural solvates (isooctane and petroleum ether solvates). The Nicotinamide cocrystal (mixture with CCF) hit from screening was found to be unstable, and five scale-up attempts for phase-pure Nicotinamide cocrystals were unsuccessful and yielded the CCF. Table 5 summarizes the attributes of several cocrystals obtained.

TABLE 5

| | | | | TGA | DSC |
|---|---|---|---|---|---|
| | | Stoichiometry | | (% | Endotherms |
| Cocrystal | Group | ¹H NMR (CCF:API) | Nature | Wt. Loss) | (Onset,° C.) |
| Tetramethylpyrazine | Group A | 0.5:1 | Non- | 0.1 | 72.8 |
| | | (Hemi-cocrystal) | solvated | | |
| | Group B | 1:1 | Non- | 0.5 | 76.3 |
| | | (Mono-cocrystal) | solvated | | |
| L-Proline | Group C | 1:1 | Non- | 0.2 | 120.7 |
| | | (Mono-cocrystal) | solvated | | |
| | Group B | 1.1:1 | Isooctane | ~4.7 | 39.2, 106.7 |
| | | (Mono-cocrystal) | solvate | | |
| | Group B | not obtained | Petroleum | ~2.1 | 32.3, 94.5, |
| | | | ether | | 102.5 |
| | | | solvate | | (exotherm), |
| | | | | | 113.1 |
| D-Proline | Group C | 1:1 | Non- | 0.2 | 119.5 |
| | | (Mono-cocrystal) | solvated | | |
| | Group B | 1.2:1 | Isooctane | ~5.7 | 35.6, 106.3 |
| | | (Mono-cocrystal) | solvate | | |
| | Group B | not obtained | Petroleum | ~3.0 | 37.6, 98.6, |
| | | | ether | | 109.2 |
| | | | solvate | | (exotherm), |
| | | | | | 114.5 |

### Example 4: Tetramethylpyrazine Cocrystals

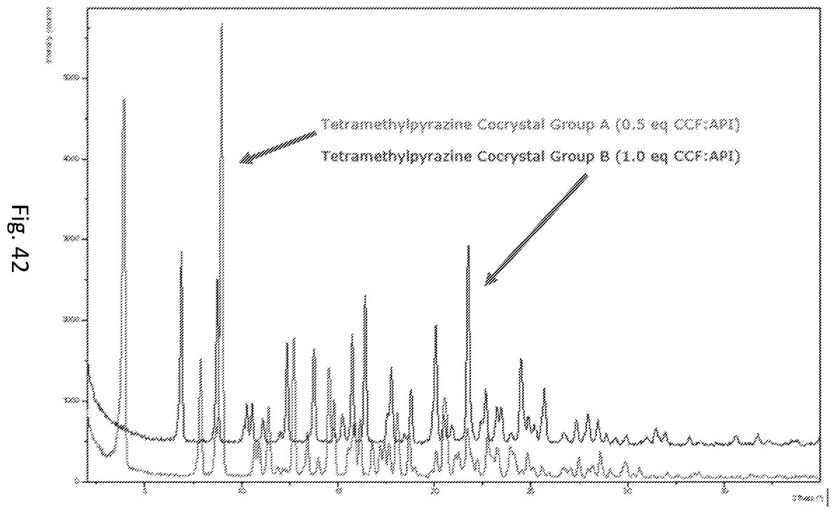

Tetramethylpyrazine cocrystal hits were obtained for Groups A and B. FIG. 42 shows an overlay of powder X-ray diffraction patterns of tetramethylpyrazine cocrystals for Groups A and B.

Preparation of Group A (Non-Solvated Tetramethylpyrazine Hemi Cocrystal)

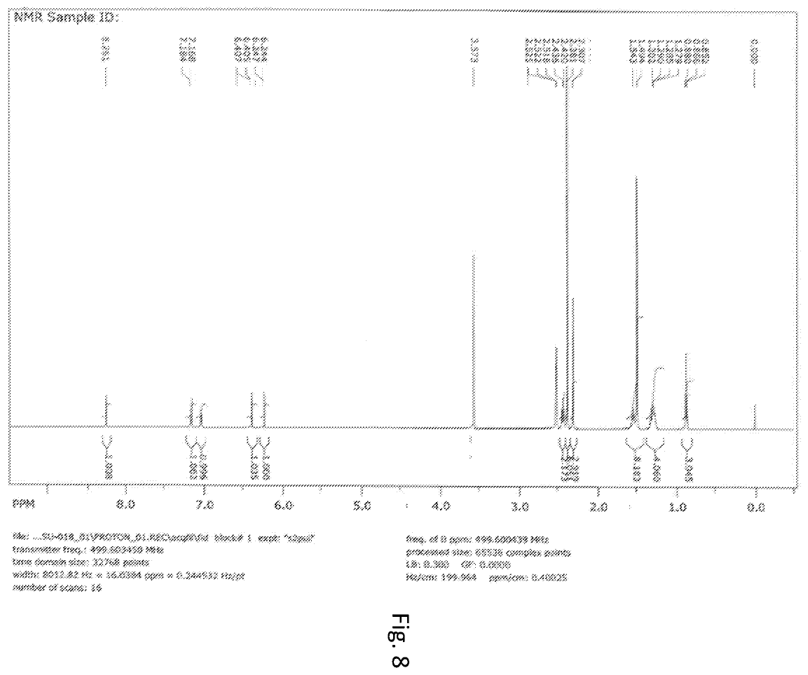

Group A is a non-solvated cocrystal of Tetramethylpyrazine/CBN (0.5:1) obtained as phase-pure using a stoichiometric ratio (0.5:1, CCF/CBN) of the reagents. The cannabinol starting material (164.9 mg) was combined with solid CCF (36.4 mg, 0.5 eq) and solvent (isooctane, 350 μL, 2.1 vol). The suspension was seeded with a mixture of Groups A and B (corresponding with seed number 10 in Table 4 in the isooctane solvent swap screening experiments) (1-2 mg) and stirred at RT for 0.5 hours forming a nonflowing suspension. More solvent (isooctane, 1.4 mL, 8.1 vol) was added and the suspension was stirred at RT for 20 hours. The product was isolated by vacuum filtration for 4 h. The weight was 133.1 mg. (67.3% cocrystal yield). The product was determined to be a crystalline powder by PLM and PXRD analyses (FIG. 6). DSC analysis showed a single sharp endotherm with onset at 72.8° C. (ΔH=85.0 J/g) (FIG. 7). For reference, the melting point of tetramethylpyrazine is 77-80° C. TGA-IR analysis showed 0.1% weight loss up to melting, indicating a non-solvated form (FIG. 7). Proton NMR analysis confirmed a 0.5:1 CCF:API stoichiometry (FIG. 8). The product was physically stable by PXRD for six days at ambient. Table 5A shows the FT-Raman Peaks (left-most column) and PXRD peak positions and intensities (middle and right-most columns) corresponding with the Non-Solvated Tetramethylpyrazine Hemi Cocrystal product Group A.

TABLE 5A

| FT- | PXRD | |
|---|---|---|
| Raman | Peak Pos, | |
| (cm⁻¹) | °2θ | Rel. Int. |
| 169 | 3.92 | 83.6 |
| 228 | 7.89 | 26.4 |
| 320 | 8.98 | 100.0 |
| 365 | 10.70 | 7.5 |
| 379 | 10.94 | 7.6 |
| 408 | 11.42 | 15.4 |
| 477 | 12.70 | 31.0 |
| 509 | 13.42 | 10.0 |
| 536 | 13.99 | 4.1 |
| 549 | 14.55 | 24.6 |
| 564 | 14.81 | 16.8 |
| 581 | 15.49 | 6.2 |
| 608 | 15.86 | 12.7 |
| 664 | 16.14 | 12.8 |
| 693 | 16.78 | 8.1 |
| 714 | 17.18 | 7.0 |
| 732 | 17.41 | 7.9 |
| 774 | 17.66 | 7.3 |
| 863 | 18.10 | 13.9 |
| 883 | 18.71 | 10.3 |
| 998 | 20.08 | 5.8 |
| 1028 | 20.47 | 16.3 |
| 1107 | 21.05 | 4.5 |
| 1156 | 21.22 | 7.4 |
| 1192 | 21.70 | 10.8 |
| 1241 | 22.23 | 3.7 |
| 1284 | 22.75 | 9.3 |
| 1297 | 23.28 | 6.4 |
| 1339 | 23.91 | 6.5 |
| 1375 | 24.81 | 5.4 |
| 1437 | 25.58 | 2.5 |
| 1497 | 27.04 | 2.2 |
| 1513 | 27.45 | 4.5 |
| 1570 | 28.57 | 5.9 |
| 1582 | 29.10 | 1.9 |
| 1617 | 29.88 | 3.4 |
| 2850 | 30.62 | 2.0 |
| 2924 | | |
| 2979 | | |
| 3039 | | |

Preparation of Group B (Non-Solvated Mono Cocrystal)

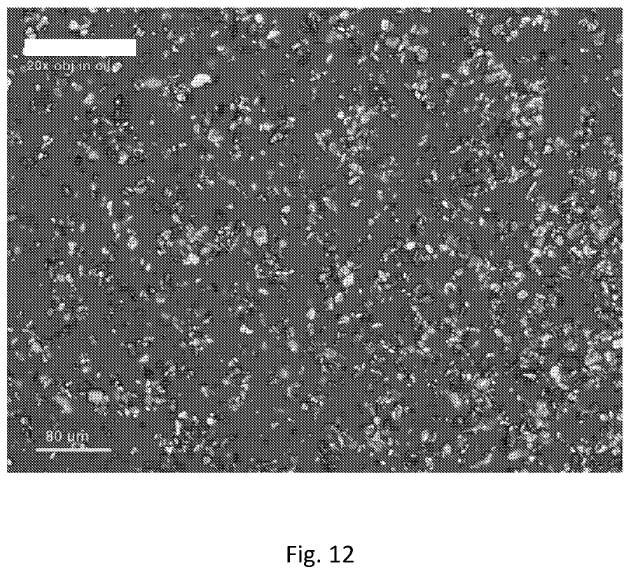

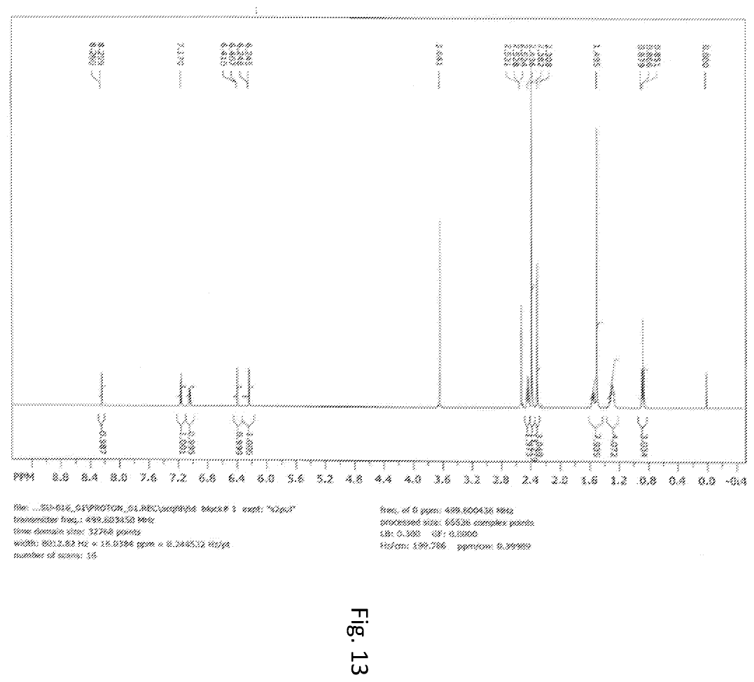

Group B is a non-solvated cocrystal of Tetramethylpyrazine/CBN (1:1) obtained as phase-pure using an excess of CCF (2:1, CCF/CBN). The cannabinol starting material (162.1 mg) was combined with solid CCF (142.6 mg, 2.0 eq) and solvent (isooctane, 650 μL, 4.0 vol). The suspension was seeded with a mixture of Groups A and B (corresponding with seed number 10 in Table 4 in the isooctane solvent swap screening experiments) (1-2 mg) and stirred at RT for 0.5 hours forming a nonflowing suspension. More solvent (isooctane, 100 μL, 0.6 vol) was added and the suspension was stirred at RT for 20 hours. The product was isolated by vacuum filtration for 4 h. The weight was 114.1 mg. (48.9% cocrystal yield). The product was determined to be a crystalline powder by PLM and PXRD analyses (FIG. 12 and FIG. 10). DSC analysis showed a single sharp endotherm with onset at 76.3° C. (ΔH=87.2 J/g) (FIG. 11). TGA-IR analysis showed 0.5% weight loss up to melting, indicating a non-solvated form (FIG. 11). Proton NMR analysis confirmed a 1:1 CCF:API stoichiometry (FIG. 13). The product was physically stable by PXRD for six days at ambient. Table 5B shows the FT-Raman Peaks (left-most column) and PXRD peak positions and intensities (middle and right-most columns) corresponding with the Non-Solvated Tetramethylpyrazine Cocrystal product Group B.

TABLE 5B

| FT- | PXRD | |
|---|---|---|
| Raman | Peak Pos, | |
| (cm⁻¹) | °2θ | Rel. Int. |
| 166 | 6.91 | 93.2 |
| 229 | 8.75 | 80.6 |
| 245 | 10.29 | 18.3 |
| 264 | 10.56 | 19.0 |
| 322 | 11.13 | 11.3 |
| 343 | 12.36 | 49.4 |
| 379 | 13.76 | 47.0 |
| 409 | 14.77 | 6.5 |
| 479 | 15.24 | 14.0 |
| 507 | 15.72 | 54.2 |
| 535 | 16.41 | 73.5 |
| 583 | 17.78 | 36.3 |
| 598 | 18.80 | 27.0 |
| 626 | 20.07 | 57.6 |
| 667 | 20.48 | 13.8 |
| 703 | 20.91 | 8.7 |
| 724 | 21.76 | 100.0 |
| 862 | 22.69 | 25.5 |
| 1027 | 23.24 | 18.2 |
| 1101 | 23.46 | 17.6 |
| 1158 | 23.95 | 5.7 |
| 1191 | 24.46 | 42.9 |
| 1225 | 24.86 | 13.9 |
| 1240 | 25.70 | 26.4 |
| 1284 | 27.32 | 11.9 |
| 1300 | 27.94 | 15.2 |
| 1342 | 28.45 | 11.5 |
| 1385 | 28.90 | 5.1 |
| 1440 | 31.45 | 8.1 |
| 1506 | 31.95 | 6.2 |
| 1549 | 35.57 | 4.2 |
| 1573 | 36.71 | 3.9 |
| 1584 | | |
| 1607 | | |
| 1618 | | |
| 2856 | | |
| 2925 | | |
| 2969 | | |
| 2987 | | |
| 3036 | | |

### Example 5: L-Proline Cocrystals

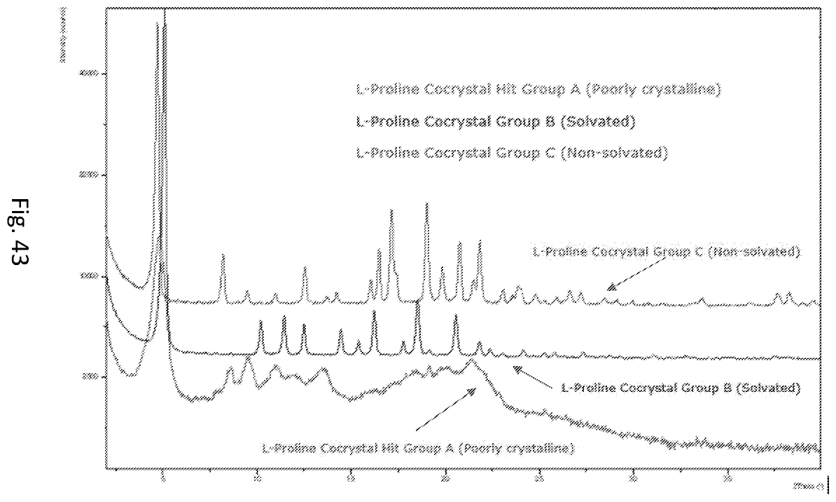

Three L-Proline cocrystal hits were obtained from the screening experiments, designated as Groups A, B, and C. Group A was determined to be poorly crystalline and unstable (exhibited deliquescence in three days at ambient). Group B was a class of isostructural solvates (isooctane and petroleum ether solvates). Group C was a non-solvated cocrystal. FIG. 43 shows an overlay of powder X-ray diffraction patterns of Groups A, B, and C of the L-Proline cocrystals.

Preparation of Group C (Mono-L-Proline Cocrystal)

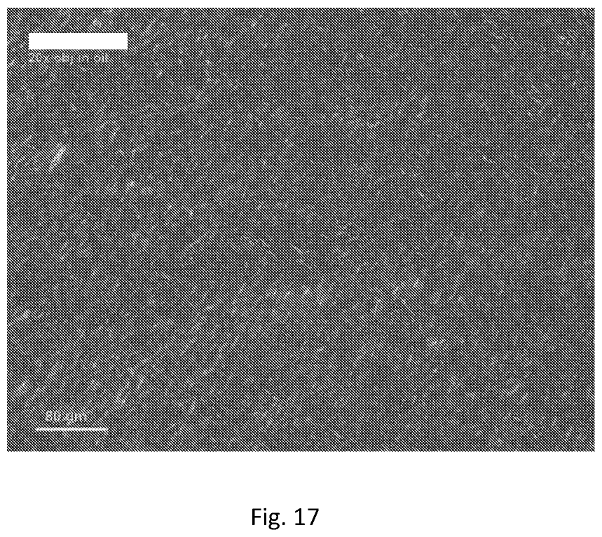

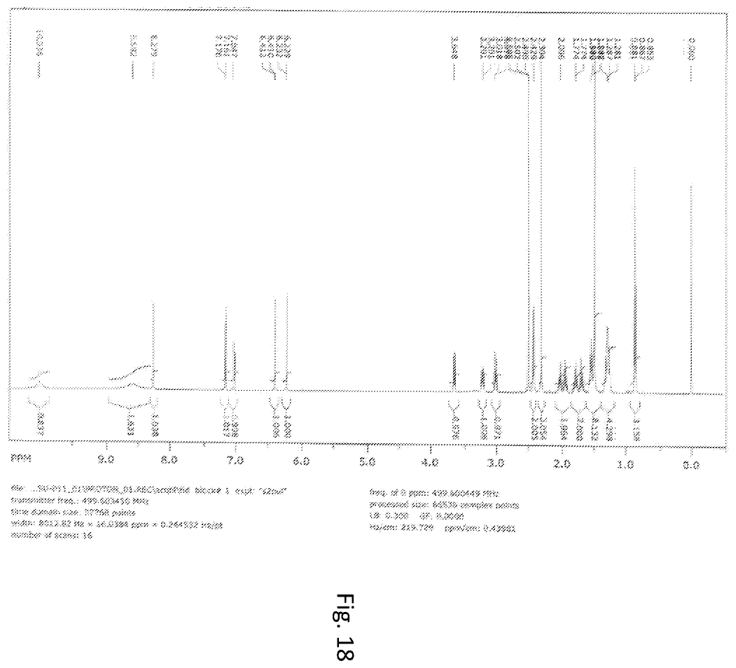

Group C is a non-solvated cocrystal of L-Proline/CBN (1:1). The cannabinol starting material (197.4 mg) was combined with solid CCF (58.8 mg, 0.8 eq) and solvent (petroleum ether, 4 mL, 20.3 vol). The suspension was seeded with 1-2 mg of the mono-L-proline cocrystal Group B isooctane solvate, (please see below for details on this preparation) and stirred and temperature-cycled between 40° C.-5° C. (1 hour at each temperature) for two days. The product was isolated by vacuum filtration for 3 h. The weight was 107.8 mg (39.8% cocrystal yield); however, the suspension was very thick, so a significant amount remained in the vial after transfer. The product was determined to be a crystalline powder by PLM and PXRD analyses (FIG. 17 and FIG. 15). DSC analysis showed a single sharp endotherm with onset at 120.7° C. (ΔH=69.9 J/g) (FIG. 16). For reference, L-proline has a melting point of about 221° C. TGA-IR analysis showed 0.2% weight loss up to melting, indicating a non-solvated form (FIG. 16). Proton NMR analysis confirmed a 1:1 CCF:API stoichiometry (FIG. 18). The product was physically stable by PXRD for three days at ambient. Table 5C shows the FT-Raman Peaks (left-most column) and PXRD peak positions and intensities (middle and right-most columns) corresponding with the Group C (Mono-L-Proline Cocrystal).

TABLE 5C

| FT- | PXRD | |
|---|---|---|
| Raman | Peak Pos, | |
| (cm⁻¹) | °2θ | Rel. Int. |
| 163 | 4.72 | 100.0 |
| 240 | 6.19 | 0.0 |
| 324 | 8.21 | 11.1 |
| 362 | 9.47 | 2.5 |
| 408 | 10.98 | 2.0 |
| 486 | 12.56 | 8.0 |
| 509 | 13.75 | 1.2 |
| 530 | 14.25 | 2.2 |
| 551 | 16.03 | 4.8 |
| 580 | 16.48 | 12.6 |
| 621 | 17.15 | 24.1 |
| 671 | 19.04 | 26.2 |
| 696 | 19.85 | 8.2 |
| 711 | 20.77 | 14.8 |
| 773 | 21.50 | 5.0 |
| 841 | 21.84 | 14.9 |
| 863 | 23.09 | 2.9 |
| 915 | 23.90 | 3.8 |
| 935 | 24.80 | 2.0 |
| 995 | 25.91 | 1.3 |
| 1027 | 26.62 | 2.9 |
| 1110 | 27.20 | 2.3 |
| 1157 | 37.69 | 2.5 |
| 1194 | 38.26 | 2.2 |
| 1236 | | |
| 1297 | | |
| 1330 | | |
| 1400 | | |
| 1444 | | |
| 1496 | | |
| 1509 | | |
| 1583 | | |
| 1615 | | |
| 2885 | | |
| 2923 | | |
| 2972 | | |
| 3020 | | |

Group B (Class of Solvates of Mono L-Proline Cocrystal)

Group B is a class of structurally similar solvates (isooctane and petroleum ether solvates) as determined by PXRD. Both solvates indicated physical instability as demonstrated by an excess of CCF showing on storage.

Preparation of Mono-L-Proline Cocrystal Group B (Isooctane Solvate)

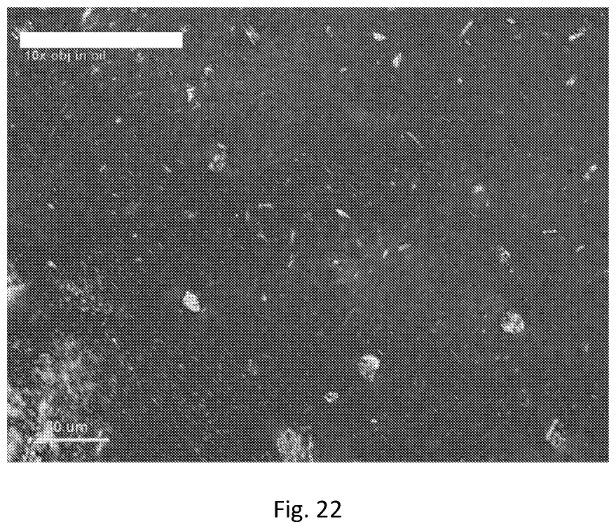

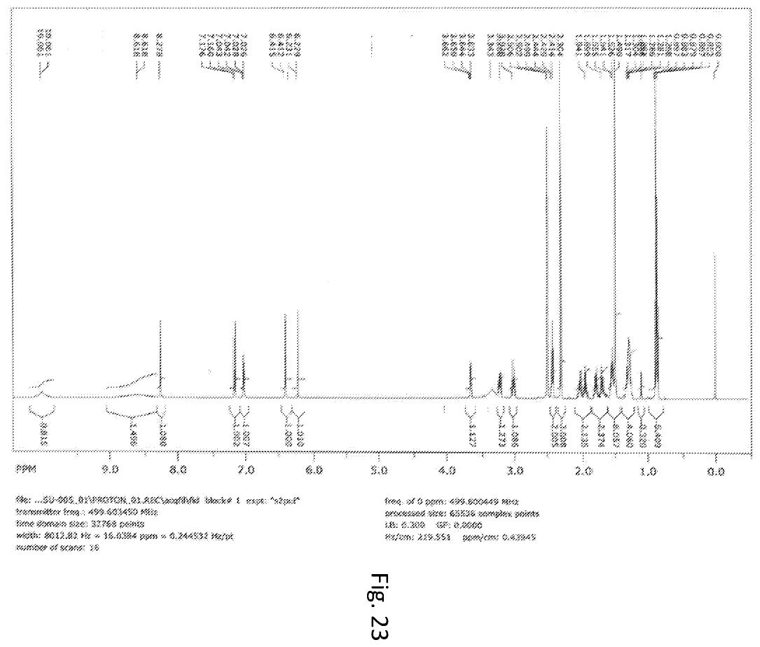

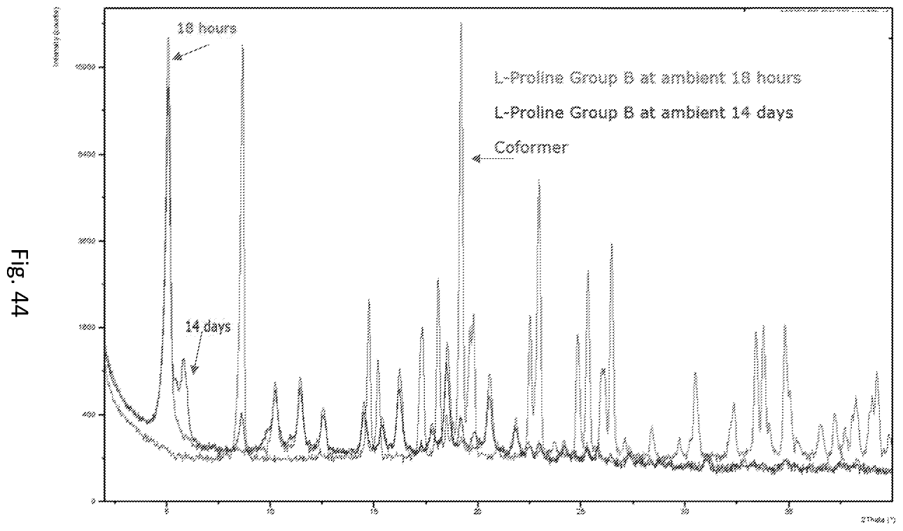

The cannabinol starting material (98.2 mg) was combined with solid CCF (36.2 mg, 1.0 eq) and solvent (isooctane, 2.0 mL, 20.4 vol). The suspension was seeded (1-2 mg of seed number 5 in Table 4 in the isooctane solvent swap screening experiments) and stirred and temperature-cycled between 40° C.-5° C. (1 hour at each temperature) for 20 hours forming a non-flowing suspension. More solvent (isooctane, 1.5 mL) was added and the suspension was stirred and temperature-cycled between 40° C.-5° C. (1 hour at each temperature) for four days. The product was isolated by vacuum filtration for 4.5 hours. The weight was 81.8 mg. (59.2% cocrystal yield); however, the suspension was very thick, so a significant amount remained in the vial after transfer. The product was determined to be a crystalline powder by PLM and PXRD analyses (FIG. 22 and FIG. 20). DSC analysis showed two broad desolvation endotherms with onsets at 39.2° C. (ΔH=20.6 J/g) and 106.7° C. (ΔH=59.5 J/g) (FIG. 21). TGA-IR analysis showed 4.7% (0.2 eq) weight loss of isooctane in two steps upon heating prior to decomposition, indicating a solvated form (FIG. 21). Proton NMR analysis confirmed a 1.1:1 CCF:API stoichiometry (FIG. 23). PXRD analysis of L-Proline Group B screening samples (e.g. seed 5 in Table 4 in the isooctane solvent swap screening experiments) exposed to ambient conditions for up to two weeks showed CCF peaks increasing and new peaks appearing, indicating some physical instability (FIG. 44). While the solvated cocrystal displayed some stability issues, it was helpful in its use as a seed crystal for preparing stable non-solvated crystals described herein.

Table 5D shows the FT-Raman Peaks (left-most column) and PXRD peak positions and intensities (middle and right-most columns) corresponding with the Mono-L-Proline Cocrystal Group B (Isooctane Solvate).

TABLE 5D

| FT- | PXRD | |
|---|---|---|
| Raman | Peak Pos, | |
| (cm⁻¹) | °2θ | Rel. Int. |
| 163 | 5.09 | 100.0 |
| 222 | 10.23 | 4.6 |
| 320 | 11.45 | 5.5 |
| 353 | 12.52 | 4.1 |
| 410 | 14.49 | 3.3 |
| 489 | 15.40 | 1.8 |
| 533 | 16.22 | 6.2 |
| 548 | 17.77 | 1.8 |
| 583 | 18.52 | 8.3 |
| 670 | 19.17 | 0.8 |
| 713 | 20.56 | 5.8 |
| 864 | 21.82 | 1.8 |
| 902 | 22.38 | 0.9 |
| 1030 | 23.05 | 0.3 |
| 1155 | 24.13 | 0.8 |
| 1194 | 25.21 | 0.5 |
| 1240 | 25.82 | 0.5 |
| 1284 | 27.32 | 0.6 |
| 1303 | | |
| 1333 | | |
| 1376 | | |
| 1405 | | |
| 1438 | | |
| 1496 | | |
| 1512 | | |
| 1573 | | |
| 1587 | | |
| 1610 | | |
| 1619 | | |
| 2850 | | |
| 2922 | | |
| 2988 | | |
| 3049 | | |

Drying of the L-Proline cocrystal Group B to 70° C. (beyond the initial desolvation endotherm) with a three minute hold at 70° C. resulted in a reduction in the size of the DSC low-temperature endotherm and a decrease in the size of the excess coformer peaks by PXRD; however, the excess CCF peaks returned over time at ambient conditions.

Preparation of L-Proline Cocrystal Group B (Petroleum Ether Solvate)

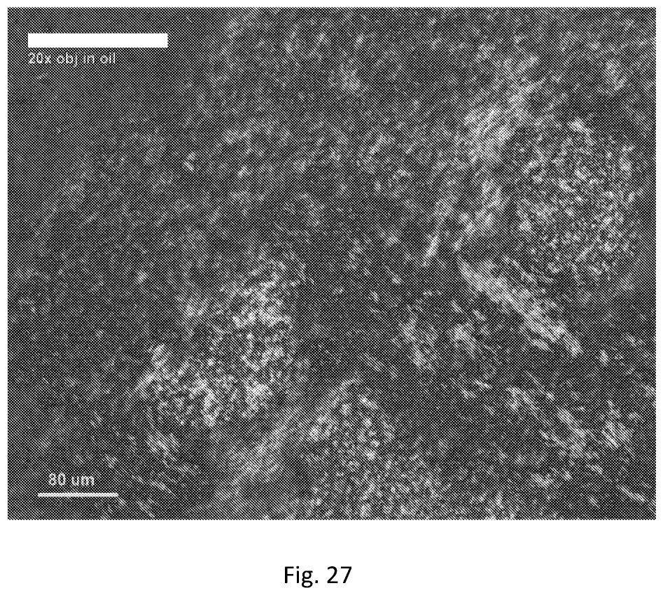

The L-Proline Cocrystal Group B—isooctane solvate (˜65 mg) was combined with solvent (petroleum ether, 5.0 mL, 76.9 vol). The suspension was stirred at 20° C. for two days. The product was isolated by vacuum filtration for 3.0 hours. The product (L-Proline Cocrystal B/petroleum ether solvate) was determined to be a crystalline powder by PXRD and PLM analyses (FIG. 25 and FIG. 27). DSC analysis showed two broad desolvation endotherms with onsets at 32.3° C. (ΔH=3.5 J/g) and 94.5° C. (ΔH=2.9 J/g), an exotherm with onset at 102.5° C. (ΔH=3.4 J/g), and a sharper endotherm with onset at 113.1° C. (ΔH=22.1 J/g) (FIG. 26). TGA-IR analysis showed 2.1% (˜0.1 eq) weight loss of petroleum ether upon heating prior to decomposition, indicating a solvated form (FIG. 26). Table 5E shows the FT-Raman Peaks (left-most column) and PXRD peak positions and intensities (middle and right-most columns) corresponding with the Mono-L-Proline Cocrystal Group B (Petroleum Ether Solvate).

TABLE 5E

| FT- | PXRD | |
|---|---|---|
| Raman | Peak Pos, | |
| (cm⁻¹) | °2θ | Rel. Int. |
| 169 | 5.09 | 100.0 |
| 224 | 10.25 | 5.2 |
| 321 | 10.81 | 0.3 |
| 410 | 11.45 | 5.8 |
| 448 | 12.53 | 1.7 |
| 489 | 14.49 | 2.3 |
| 531 | 15.17 | 7.5 |
| 548 | 16.21 | 6.0 |
| 582 | 18.07 | 17.6 |
| 606 | 18.51 | 10.3 |
| 623 | 19.60 | 8.4 |
| 669 | 20.55 | 5.5 |
| 694 | 21.82 | 1.7 |
| 712 | 22.80 | 1.1 |
| 842 | 24.80 | 5.6 |
| 863 | 25.83 | 0.7 |
| 899 | 27.18 | 0.5 |
| 919 | 30.61 | 1.1 |
| 951 | 32.18 | 0.9 |
| 993 | | |
| 1030 | | |
| 1056 | | |
| 1155 | | |
| 1193 | | |
| 1239 | | |
| 1284 | | |
| 1302 | | |
| 1333 | | |
| 1376 | | |
| 1404 | | |
| 1444 | | |
| 1511 | | |
| 1572 | | |
| 1585 | | |
| 1609 | | |
| 1618 | | |
| 2931 | | |
| 2984 | | |
| 3001 | | |

Drying of the L-Proline cocrystal Group B in a vacuum oven for three days was conducted to verify if a non-solvated form could be obtained. The drying resulted in a reduction in the solvent content from 2.1% to 0.8% by TGA-IR; however, the DSC trace and PXRD pattern did not change.

### Example 6: D-Proline Cocrystals

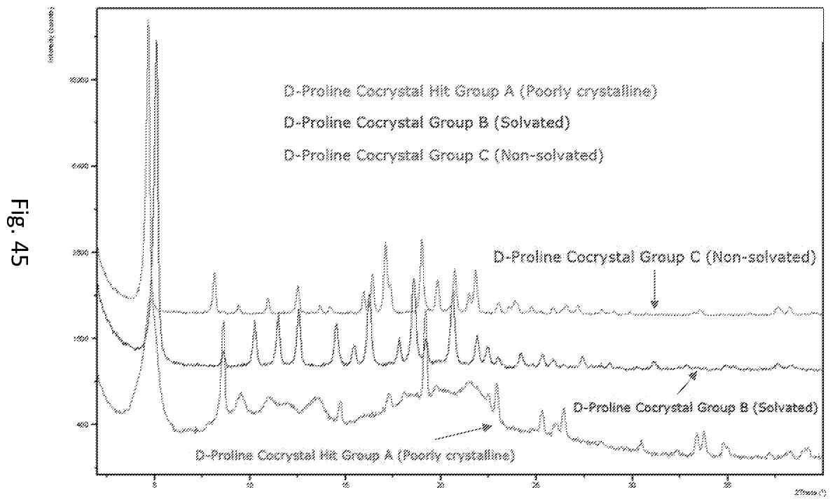

Three D-Proline cocrystal hits were obtained from screening, designated as Groups A, B, and C. Group A was determined to be poorly crystalline and unstable (exhibited deliquescence in three days at ambient). Group B is a class of isostructural solvates (isooctane and petroleum ether solvates). Group C is a non-solvated cocrystal. FIG. 45 shows an overlay of Groups A, B, and C of the D-Proline cocrystal.

Preparation of Mono-D-Proline Cocrystal Group C (Non-Solvated)

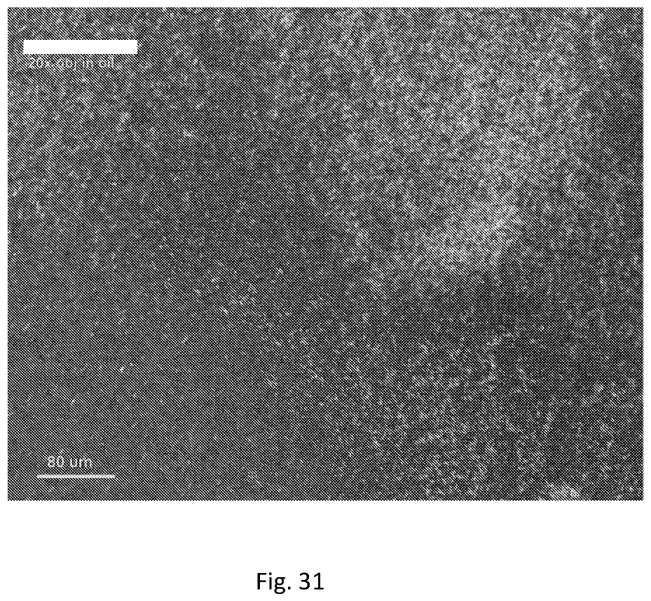

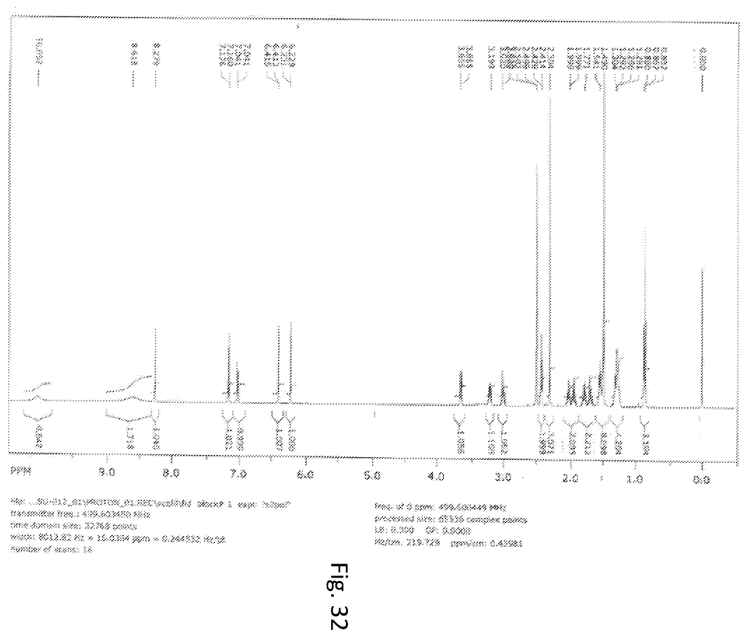

Group C is a non-solvated cocrystal of D-Proline/CBN (1:1). The cannabinol starting material (203.9 mg) was combined with solid CCF (60.4 mg, 0.8 eq) and solvent (petroleum ether, 2.5 mL, 12.3 vol). The suspension was seeded with 1-2 mg of the mono-D-proline cocrystal Group B isooctane solvate (see below for information on its preparation) and stirred and temperature-cycled 40° C.-5° C. (1 hour at each temperature) for two days. The product was isolated by vacuum filtration for 2.5 hours. The weight was 82.8 mg (29.6% cocrystal yield); however, the suspension was very thick, so a significant amount remained in the vial after transfer. The product was determined to be a crystalline powder by PXRD and PLM analyses (FIG. 29 and FIG. 31). DSC analysis showed a single sharp endotherm with onset at 119.5° C. (ΔH=68.5 J/g) (FIG. 30). For reference, the melting point of D-proline is about 223° C. TGA-IR analysis showed 0.2% weight loss up to melting, indicating a non-solvated form (FIG. 30). Proton NMR analysis confirmed a 1:1 CCF:API stoichiometry (FIG. 32). The product was physically stable by PXRD for three days at ambient. Table 5F shows the FT-Raman Peaks (left-most column) and PXRD peak positions and intensities (middle and right-most columns) corresponding with the Mono-D-Proline Cocrystal Group C (Non-solvated).

TABLE 5F

| FT- | PXRD | |
|---|---|---|
| Raman | Peak Pos, | |
| (cm⁻¹) | °2θ | Rel. Int. |
| 163 | 4.70 | 100.0 |
| 239 | 8.18 1 | 9.0 |
| 323 | 9.44 | 1.6 |
| 362 | 10.95 | 3.0 |
| 408 | 12.53 | 5.6 |
| 486 | 15.99 | 4.5 |
| 508 | 16.44 | 8.4 |
| 531 | 17.11 | 16.2 |
| 551 | 19.01 | 17.0 |
| 580 | 19.82 | 7.2 |
| 621 | 20.74 | 9.5 |
| 671 | 21.46 | 4.0 |
| 696 | 21.81 | 9.5 |
| 711 | 23.01 | 2.3 |
| 773 | 23.93 | 2.4 |
| 841 | 24.77 | 1.4 |
| 863 | 26.55 | 2.7 |
| 915 | 27.14 | 1.5 |
| 995 | 37.64 | 1.5 |
| 1027 | 38.24 | 1.4 |
| 1110 | | |
| 1157 | | |
| 1193 | | |
| 1236 | | |
| 1298 | | |
| 1329 | | |
| 1399 | | |
| 1443 | | |
| 1508 | | |
| 1583 | | |
| 1615 | | |
| 2924 | | |
| 2985 | | |
| 3021 | | |

Group B (Class of Solvates of Mono D-Proline Cocrystal)

Group B is a class of structurally similar solvates (isooctane and petroleum ether solvates) by PXRD. Both solvates indicated physical instability as demonstrated by an excess of CCF showing on storage.

Preparation of Mono-D-Proline Cocrystal Group B (Isooctane Solvate)

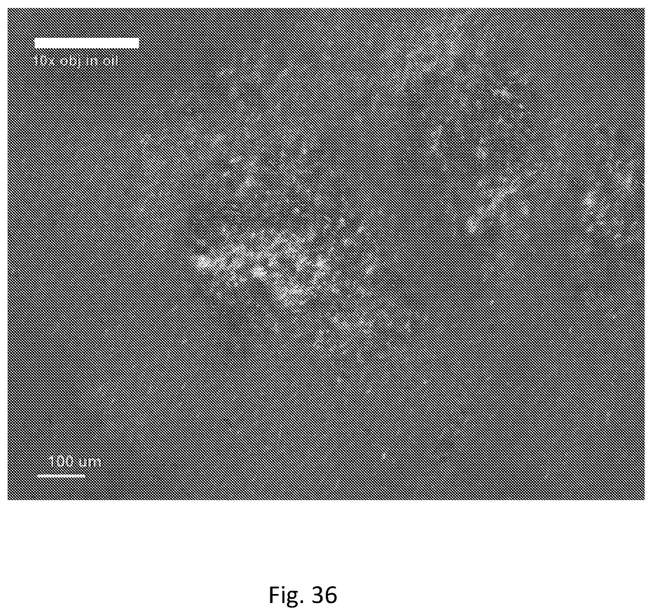

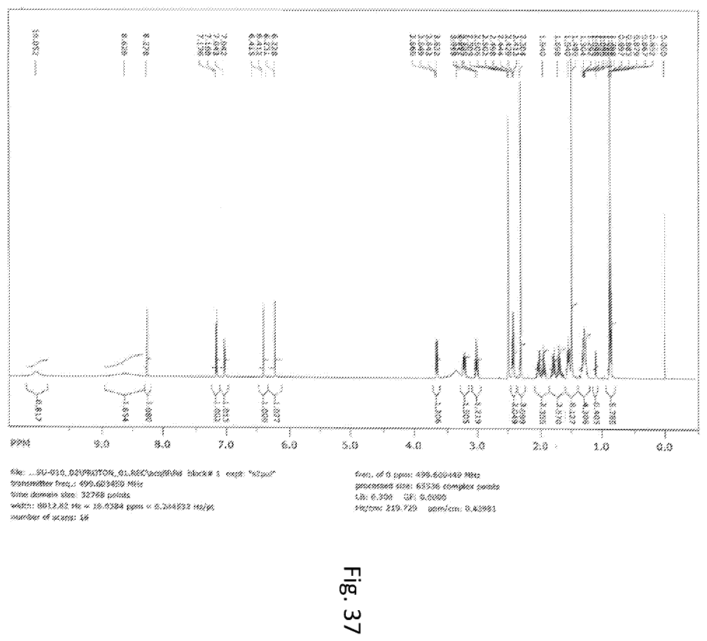

The cannabinol starting material (106.0 mg) was combined with solid CCF (39.4 mg, 1.0 eq) and solvent (isooctane, 6.0 mL, 56.6 vol). The suspension was seeded (1-2 mg of seed number 6 in Table 4 in the isooctane solvent swap screening experiments) and stirred at 40° C. for four hours, cooled at 0.1° C./min to 20° C. and stirred for 16 hours, then stirred and temperature-cycled between 40° C.-5° C. (1 hour at each temperature) for 72 hours. The product was isolated by vacuum filtration for 3.5 hours. The weight was 99.7 mg (65.1% cocrystal yield). The product was determined to be a crystalline powder by PXRD and PLM analyses (FIG. 34 and FIG. 36). DSC analysis showed two broad desolvation endotherms with onsets at 35.6° C. (ΔH=14.7 J/g) and 106.3° C. (ΔH=59.2 J/g) (FIG. 35). TGA-IR analysis showed 5.7% (0.24 eq) weight loss of isooctane in two steps upon heating prior to decomposition, indicating a solvated form (FIG. 35). Proton NMR analysis confirmed a 1.2:1 CCF:API stoichiometry (FIG. 37). PXRD analysis of D-Proline Group B screening samples exposed to ambient conditions for up to two weeks showed CCF peaks increasing and new peaks appearing, indicating some instability. While the solvated cocrystal displayed some stability issues, it was helpful in its use as a seed crystal for preparing stable non-solvated crystals described herein.

Table 5G shows the FT-Raman Peaks (left-most column) and PXRD peak positions and intensities (middle and right-most columns) corresponding with the Mono-D-Proline Cocrystal Group B (Isooctane Solvate).

TABLE 5G

| FT- | PXRD | |
|---|---|---|
| Raman | Peak Pos, | |
| (cm⁻¹) | °2θ | Rel. Int. |
| 168 | 5.13 | 100.0 |
| 224 | 8.65 | 2.2 |
| 242 | 10.28 | 7.5 |
| 320 | 11.50 | 9.1 |
| 350 | 12.58 | 10.2 |
| 409 | 14.54 | 7.3 |
| 489 | 15.46 | 3.3 |
| 532 | 16.28 | 13.5 |
| 548 | 17.84 | 4.5 |
| 583 | 18.58 | 16.9 |
| 623 | 19.22 | 4.4 |
| 670 | 20.63 | 14.2 |
| 712 | 21.89 | 5.0 |
| 743 | 22.47 | 3.1 |
| 830 | 24.14 | 2.0 |
| 863 | 25.31 | 2.0 |
| 900 | 25.87 | 1.1 |
| 921 | 27.41 | 1.6 |
| 931 | 38.24 | 1.4 |
| 1029 | | |
| 1154 | | |
| 1194 | | |
| 1239 | | |
| 1283 | | |
| 1303 | | |
| 1333 | | |
| 1348 | | |
| 1376 | | |
| 1404 | | |
| 1437 | | |
| 1496 | | |
| 1512 | | |
| 1572 | | |
| 1587 | | |
| 1609 | | |
| 1619 | | |
| 2869 | | |
| 2922 | | |
| 2988 | | |
| 3049 | | |

Preparation of D-Proline Cocrystal Group B (Petroleum Ether Solvate)

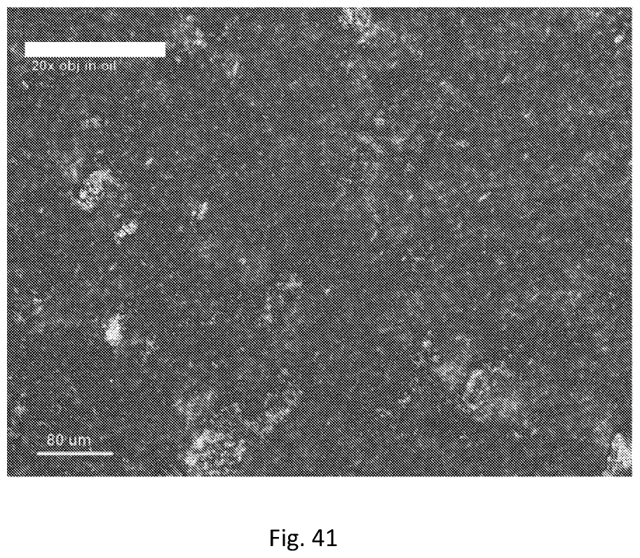

The D-Proline Cocrystal Group B—isooctane solvate (80.0 mg) was combined with solvent (petroleum ether, 2.0 mL, 25.0 vol). The suspension was stirred and temperature-cycled between 40° C.-5° C. (1 hour at each temperature) for two days. The product was isolated by vacuum filtration for 2.0 hours. The product (D-Proline Cocrystal Group B (Petroleum Ether Solvate)) was determined to be a crystalline powder by PXRD and PLM analyses (FIG. 39 and FIG. 41). DSC analysis showed two broad desolvation endotherms with onsets at 37.6° C. (ΔH=25.3 J/g) and 98.6° C. (ΔH=2.0 J/g), an exotherm with onset at 109.2° C. (ΔH=0.8 J/g) and a sharper endotherm with onset at 114.5° C. (ΔH=32.6 J/g) (FIG. 40). TGA-IR analysis showed 3.0% (0.12 eq) weight loss of petroleum ether upon heating prior to decomposition, indicating a solvated form (FIG. 40). Table 5H shows the FT-Raman Peaks (left-most column) and PXRD peak positions and intensities (middle and right-most columns) corresponding with the Mono-D-Proline Cocrystal Group B (Petroleum Ether Solvate).

TABLE 5H

| FT- | PXRD | |
|---|---|---|
| Raman | Peak Pos, | |
| (cm⁻¹) | °2θ | Rel. Int. |
| 164 | 5.12 | 100.0 |
| 224 | 8.64 | 6.8 |
| 320 | 10.26 | 6.1 |
| 409 | 11.48 | 7.9 |
| 489 | 12.54 | 2.9 |
| 532 | 14.52 | 3.3 |
| 548 | 14.83 | 0.3 |
| 583 | 15.42 | 3.8 |
| 623 | 16.24 | 7.9 |
| 670 | 17.79 | 2.1 |
| 712 | 18.55 | 12.3 |
| 863 | 19.18 | 3.1 |
| 903 | 20.58 | 7.6 |
| 1029 | 21.83 | 2.4 |
| 1154 | 22.99 | 0.8 |
| 1193 | 24.17 | 1.0 |
| 1239 | 25.27 | 0.8 |
| 1283 | 25.80 | 0.8 |
| 1302 | 27.33 | 0.9 |
| 1332 | | |
| 1376 | | |
| 1404 | | |
| 1437 | | |
| 1510 | | |
| 1572 | | |
| 1585 | | |
| 1609 | | |
| 1618 | | |
| 2921 | | |
| 2986 | | |

### Example 7: Nicotinamide Cocrystal Experiments

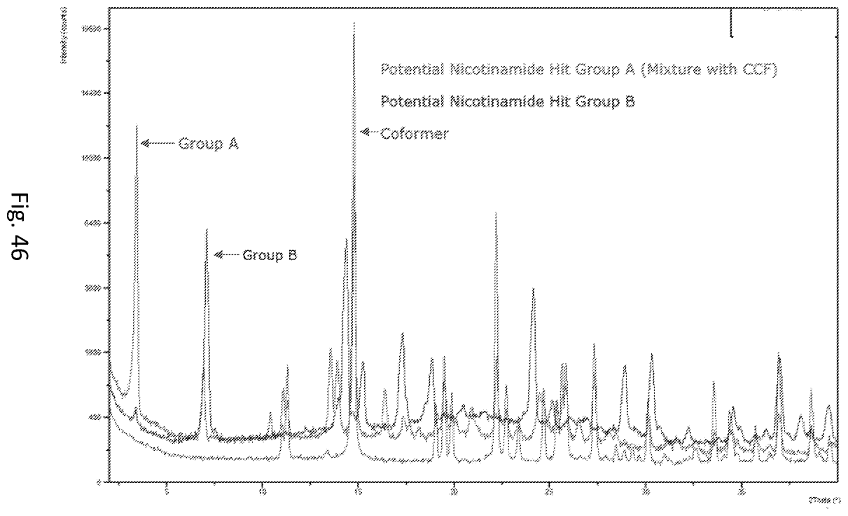

Possible Nicotinamide cocrystal hits were obtained from solid form screening. PXRD analysis indicated two forms, designated as Groups A and B, as shown in FIG. 46.

Group A was determined to be a mixture of a potential cocrystal with excess CCF; however, isolation and analysis of the residual suspension after 19 days showed only CCF. Five scale-up attempts to reproduce Nicotinamide cocrystal hit Group A involving 1, 2, and 4 equivalents CCF, three with seeding, were unsuccessful and yielded only the CCF.

Group B was determined to be a potential unstable cocrystal. Initially, the PXRD pattern of Nicotinamide cocrystal hit Group B was unique and showed no excess CCF peaks; however, after three days at ambient in the solid state, Nicotinamide cocrystal hit Group B exhibited complete conversion to the CCF.

### Example 8: Detailed Synthesis of Seed Number 6 in Table 4 in Isooctane

The cannabinol starting material (2530. mg) was dissolved in methanol and made up to volume in a 25 mL volumetric flask to yield a 101.2 mg/mL API solution. This API solution (200 μL) was pipetted into a 2 ml vial, and the solvent was rapidly evaporated under reduced pressure on a vacuum centrifuge (GeneVac®) for 18 hours, yielding ˜20.2 mg of API in the vial. Solvent (cyclohexane, 100 μL) was added to the vial containing API forming a solution. Cocrystal former (D-proline, 3 molar aqueous solution, 1 equivalent, 21.7 μL) was added forming a gum/oil mixture. The sample was stirred and temperature-cycled 40° C.-5° C. (1 hour at each temperature) for 72 hours, but remained a gum/oil mixture. The solvent (cyclohexane and water) was rapidly evaporated under reduced pressure on a vacuum centrifuge (GeneVac®) for 19 hours, forming an amorphous amber gum. Neat solvent (cyclohexane, 100 μL) was added back forming a gum/solution mixture. The sample was stirred and temperature-cycled 40° C.-5° C. (1 hour at each temperature) for 2 weeks with occasional scratching of the inside wall of the vial, but remained a gum/solution mixture. The solvent was slowly evaporated in a nitrogen flow chamber for 4 days, forming an amorphous amber gum. New solvent (isooctane, 650 μL) was added, and the sample was stirred and temperature-cycled 40° C.-5° C. (1 hour at each temperature) for 48 hours with occasional scratching of inside the wall of the vial, yielding a birefringent suspension. The solids were isolated by vacuum filtration under a nitrogen tent for 2 hours. PXRD analysis showed D-proline cocrystal Group B with excess cocrystal former.

### Example 9: Detailed Synthesis of Seed Number 5 in Table 4

The cannabinol starting material (2530. mg) was dissolved in methanol and made up to volume in a 25 mL volumetric flask to yield a 101.2 mg/mL API solution. This API solution (200 μL) was pipetted into a 2 ml vial, and the solvent was rapidly evaporated under reduced pressure on a vacuum centrifuge (GeneVac®) for 18 hours, yielding ˜20.2 mg of API in the vial. Solvent (cyclohexane, 100 μL) was added to the vial containing API forming a solution. Cocrystal former (L-proline, 3 molar aqueous solution, 1 equivalent, 21.7 μL) was added forming a gum/oil mixture. The sample was stirred and temperature-cycled 40° C.-5° C. (1 hour at each temperature) for 72 hours, but remained a gum/oil mixture. The solvent (cyclohexane and water) was rapidly evaporated under reduced pressure on a vacuum centrifuge (GeneVac®) for 19 hours, forming an amorphous amber gum. Neat solvent (cyclohexane, 100 μL) was added back forming a gum/solution mixture. The sample was stirred and temperature-cycled 40° C.-5° C. (1 hour at each temperature) for 2 weeks with occasional scratching of the inside wall of the vial, but remained a gum/solution mixture. The solvent was slowly evaporated in a nitrogen flow chamber for 4 days, forming an amorphous amber gum. New solvent (isooctane, 450 μL) was added, and the sample was stirred and temperature-cycled 40° C.-5° C. (1 hour at each temperature) for 48 hours with occasional scratching of the inside wall of the vial, yielding a birefringent suspension. The solids were isolated by vacuum filtration under a nitrogen tent for 2 hours. PXRD analysis showed L-proline cocrystal Group B with excess cocrystal former.

### Example 10: Detailed Synthesis of Seed Number 10 in Table 4

The cannabinol starting material (2530 mg) was dissolved in methanol and made up to volume in a 25 mL volumetric flask to yield a 101.2 mg/mL API solution. This API solution (200 μL) was pipetted into a 2 ml vial, and the solvent was rapidly evaporated under reduced pressure on a vacuum centrifuge (GeneVac®) for 18 hours, yielding ˜20.2 mg of API in the vial. Solvent (cyclohexane, 100 μL) was added to the vial containing API forming a solution. Cocrystal former (tetramethylpyrazine, 3 molar methanol solution, 4 equivalents, 86.8 μL) was added forming a solution. The sample was stirred and temperature-cycled 40° C.-5° C. (1 hour at each temperature) for 72 hours, but remained a solution. The solvent (cyclohexane and methanol) was rapidly evaporated under reduced pressure on a vacuum centrifuge (GeneVac®) for 19 hours, forming an amorphous amber gum. One solvent (cyclohexane, 100 μL) was added back forming a solution. The sample was stirred and temperature-cycled 40° C.-5° C. (1 hour at each temperature) for 48 hours with occasional scratching of inside wall of vial but remained a solution. The solvent was slowly evaporated in a nitrogen flow chamber for 13 days with occasional scratching of inside wall of vial, forming an amorphous amber gum. New solvent (isooctane, 50 μL) was added, and the sample was stirred and temperature-cycled 40° C.-5° C. (1 hour at each temperature) for 48 hours, with occasional scratching of inside wall of vial, forming a gum with some birefringent solids. The sample was sonicated 6 times for 30 minutes each with ˜10 minutes between sessions to allow for cooling to room temperature, forming a gum with many birefringent solids. The sample was seeded (batch 103363-SS-SW-035, D-proline Group B, 1-2 mg), stirred and temperature-cycled 25° C.-5° C. (1 hour at each temperature) for 7 days with occasional scratching of inside wall of vial yielding a birefringent suspension/gum mixture. The solids were isolated by vacuum filtration under a nitrogen tent for 2 hours. PXRD analysis showed a mixture of tetramethylpyrazine cocrystal Groups A and B.

### Example 11: Detailed Synthesis of D-Proline Screening Hit

The cannabinol starting material (2530 mg) was dissolved in methanol and made up to volume in a 25 mL volumetric flask to yield a 101.2 mg/mL API solution. This API solution (200 μL) was pipetted into a 2 ml vial, and the solvent was rapidly evaporated under reduced pressure on a vacuum centrifuge (GeneVac®) for 18 hours, yielding ˜20.2 mg of API in the vial. Solvent (heptane, 100 μL) was added to the vial containing API forming a solution. Cocrystal former (D-proline, 3 molar aqueous solution, 1 equivalent, 21.7 μL) was added forming a gum/oil mixture. The sample was stirred and temperature-cycled 40° C.-5° C. (1 hour at each temperature) for 72 hours, but remained a gum/oil mixture. The solvent (heptane and water) was rapidly evaporated under reduced pressure on a vacuum centrifuge (GeneVac®) for 19 hours, forming an amorphous amber gum. Neat solvent (heptane, 100 μL) was added back forming a gum/solution mixture. The sample was stirred and temperature-cycled 40° C.-5° C. (1 hour at each temperature) for 2 weeks with occasional scratching of the inside wall of the vial, but remained a gum/solution mixture. The solvent was slowly evaporated in a nitrogen flow chamber for 4 days, forming an amorphous amber gum. New solvent (petroleum ether, 750 μL) was added, and the sample was stirred and temperature-cycled 40° C.-5° C. (1 hour at each temperature) for 48 hours with occasional scratching of the inside wall of the vial, yielding a birefringent suspension. The solids were isolated by vacuum filtration under a nitrogen tent for 2 hours. PXRD analysis showed D-proline cocrystal Group B with excess cocrystal former.

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the disclosure and are encompassed by the appended claims.

Citation or identification of any reference in this application is not an admission that such reference is available as prior art.

Efforts have been made to ensure accuracy with respect to numbers used (e.g. amounts, temperature, etc.) but some experimental errors and deviations should be accounted for.

When an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

Where a range of values is provided, it is understood that each intervening value, to the tenth of the unit of the lower limit, unless the context clearly dictates otherwise, between the upper and lower limit of the range and any other stated or intervening value in that stated range, is encompassed. The upper and lower limits of these small ranges which may independently be included in the smaller ranges is also encompassed, subject to any specifically excluded limit in the stated range. Where the stated range includes one or both of the limits, ranges excluding either or both of those included limits are also included.

Unless defined otherwise, technical and scientific terms used herein have the same meaning as commonly understood by one of ordinary skill in the art to which this subject matter belongs, and are consistent with: Singleton et al (1994) Dictionary of Microbiology and Molecular Biology, 2nd Ed., J. Wiley & Sons, New York, NY; and Janeway, C., Travers, P., Walport, M., Shlomchik (2001) Immunobiology, 5th Ed., Garland Publishing, New York.

The disclosures of all cited references including publications, patents, and patent applications are expressly incorporated herein by reference in their entirety.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

As used herein, the term “about,” when referring to a value is meant to encompass variations of, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Many modifications and other embodiments set forth herein will come to mind to one skilled in the art to which this subject matter pertains having the benefit of the teachings presented in the foregoing descriptions and the associated drawings. Therefore, it is to be understood that the subject matter is not to be limited to the specific embodiments disclosed and that modifications and other embodiments are intended to be included within the scope of the appended claims. Although specific terms are employed herein, they are used in a generic and descriptive sense only and not for purposes of limitation. One skilled in the art will recognize many methods and materials similar or equivalent to those described herein, which could be used in the practicing the subject matter described herein. The present disclosure is in no way limited to just the methods and materials described.

1. A solid form comprising a coformer and a cannabinoid, wherein the cannabinoid is a non-solid material selected from the group consisting of cannabinol and tetrahydrocannabinol, and the conformer is selected from the group consisting of potassium, calcium, carnitine, aspartame, L-proline, D-proline, L-Arginine, L-Lysine, betaine, tetramethylpyrazine, 1H-Imidazole, nicotinic acid, saccharin, urea, and nicotinamide.

2. The solid form of claim 1, wherein the cannabinoid is an oil.

3-4. (canceled)

5. The solid form of claim 1, wherein the cannabinoid is cannabinol the coformer is tetramethylpyrazine, and the molar ratio of cannabinol to tetramethylpyrazine is about 1 to 0.5 or about 1 to 1,
wherein said solid form is crystalline or a cocrystal, substantially free of solvent, and stable.

6. (canceled)

7. The solid form of claim 5, wherein the molar ratio of cannabinol to tetramethyl pyrazine is about 1 to 0.5 and said solid form is characterized by a powder X-ray diffraction pattern comprising at least one peak, or optionally at least two peaks, or optionally at least three peaks, selected from the group consisting of 3.92, 7.89, 8.98, 10.70, 10.94, 11.42, 12.70, 13.42, 13.99, 14.55, 14.81, 15.49, 15.86, 16.14, 16.78, 17.18, 17.41, 17.66, 18.10, 18.71, 20.08, 20.47, 21.05, 21.22, 21.70, 22.23, 22.75, 23.28, 23.91, 24.81, 25.58, 27.04, 27.45, 28.57, 29.10, 29.88, and 30.62 (each degrees 2θ±0.20).

8-10. (canceled)

11. The solid form of claim 5, wherein the molar ratio of cannabinol to tetramethyl pyrazine is about 1 to 0.5 and said solid form is characterized by a DSC thermogram with a peak onset at about 72.8° C. and a peak maximum at about 74.3° C.

12. (canceled)

13. The solid form of claim 5, wherein the molar ratio of cannabinol to tetramethyl pyrazine is about 1 to 0.5 and said solid form is characterized by a Raman spectrum comprising at least one peak selected from the group consisting of 169, 228, 320, 365, 379, 408, 477, 509, 536, 549, 564, 581, 608, 664, 693, 714, 732, 774, 863, 883, 998, 1028, 1107, 1156, 1192, 1241, 1284, 1297, 1339, 1375, 1437, 1497, 1513, 1570, 1582, 1617, 2850, 2924, 2979, and 3039 (each cm⁻¹).

14-19. (canceled)

20. The solid form of claim 5, wherein the molar ratio of cannabinol to tetramethylpyrazine is about 1 to 1 and wherein said solid form is characterized by a powder X-ray diffraction pattern comprising at least one peak, or optionally at least two peaks, or optionally at least three peaks, selected from the group consisting of 6.91, 8.75, 10.29, 10.56, 11.13, 12.36, 13.76, 14.77, 15.24, 15.72, 16.41, 17.78, 18.80, 20.07, 20.48, 20.91, 21.76, 22.69, 23.24, 23.46, 23.95, 24.46, 24.86, 25.70, 27.32, 27.94, 28.45, 28.90, 31.45, 31.95, 35.57, and 36.71 (each degrees 2θ±0.20).

21-23. (canceled)

24. The solid form of claim 5, wherein the molar ratio of cannabinol to tetramethylpyrazine is about 1 to 1 and wherein said solid form is characterized by a DSC thermogram with a peak onset at about 76.3° C. and a peak maximum at about 77.8° C.

25. (canceled)

26. The solid form of claim 5, wherein the molar ratio of cannabinol to tetramethylpyrazine is about 1 to 1 and wherein said solid form is characterized by a Raman spectrum comprising at least one peak selected from the group consisting of 166, 229, 245, 264, 322, 343, 379, 409, 479, 507, 535, 583, 598, 626, 667, 703, 724, 862, 1027, 1101, 1158, 1191, 1225, 1240, 1284, 1300, 1342, 1385, 1440, 1506, 1549, 1573, 1584, 1607, 1618, 2856, 2925, 2969, 2987, and 3036 (each cm⁻¹).

27-31. (canceled)

32. The solid form of claim 1, wherein the cannabinoid is cannabinol, the coformer is L-proline, and the molar ratio of cannabinol to L-proline is about 1 to 1,
wherein said solid form is crystalline or a cocrystal, substantially free of solvent, and stable.

33. (canceled)

34. The solid form of claim 32, characterized by a powder X-ray diffraction pattern comprising at least one peak, or optionally at least two peaks, or optionally at least three peaks, selected from the group consisting of 4.72, 6.19, 8.21, 9.47, 10.98, 12.56, 13.75, 14.25, 16.03, 16.48, 17.15, 19.04, 19.85, 20.77, 21.50, 21.84, 23.09, 23.90, 24.80, 25.91, 26.62, 27.20, 37.69, and 38.26 (each degrees 2θ±0.20).

35-37. (canceled)

38. The solid form of claim 32, characterized by a DSC thermogram with a peak onset at about 120.7° C. and a peak maximum at about 124.3° C.

39. (canceled)

40. The solid form of claim 32, characterized by a Raman spectrum comprising at least one peak selected from the group consisting of 163, 240, 324, 362, 408, 486, 509, 530, 551, 580, 621, 671, 696, 711, 773, 841, 863, 915, 935, 995, 1027, 1110, 1157, 1194, 1236, 1297, 1330, 1400, 1444, 1496, 1509, 1583, 1615, 2885, 2923, 2972, and 3020 (each cm⁻¹).

41-45. (canceled)

46. The solid form of claim 1, wherein the cannabinoid is cannabinol and the coformer is D-proline, and the molar ratio of cannabinol to D-proline is about 1 to 1,
wherein said solid form is crystalline or a cocrystal, substantially free of solvent, and stable.

47. (canceled)

48. The solid form of claim 46, characterized by a powder X-ray diffraction pattern comprising at least one peak, or optionally at least two peaks, or optionally at least three peaks, selected from the group consisting of 4.70, 8.18, 9.44, 10.95, 12.53, 15.99, 16.44, 17.11, 19.01, 19.82, 20.74, 21.46, 21.81, 23.01, 23.93, 24.77, 26.55, 27.14, 37.64, and 38.24 (each degrees 2θ±0.20).

49-51. (canceled)

52. The solid form of claim 46, characterized by a DSC thermogram with a peak onset at about 119.5° C. and a peak maximum at about 125.3° C.

53. (canceled)

54. The solid form of claim 46, characterized by a Raman spectrum comprising at least one peak selected from the group consisting of 163, 239, 323, 362, 408, 486, 508, 531, 551, 580, 621, 671, 696, 711, 773, 841, 863, 915, 995, 1027, 1110, 1157, 1193, 1236, 1298, 1329, 1399, 1443, 1508, 1583, 1615, 2924, 2985, and 3021 (each cm⁻¹).

55-59. (canceled)

60. A pharmaceutical composition comprising the solid form of claim 1, and a pharmaceutically acceptable excipient.

61. (canceled)

62. A method of preparing a solid form comprising a coformer and a cannabinoid, starting from a cannabinoid oil selected from the group consisting of cannabinol and tetrahydrocannabinol, said method comprising:
contacting said cannabinoid with said coformer and a solvent to prepare a suspension, wherein said solvent is selected from the group consisting of isooctane, petroleum ether, methanol, isopropanol, acetonitrile, acetone, tetrahydrofuran, 1,4-dioxane, ethyl acetate, 4-methyl-2-pentanone, dichloromethane, methyl t-butyl ether, and toluene;
seeding said suspension with a seed cocrystal;
heating said suspension at a first temperature with stirring; and,
separating a solid material from said suspension, wherein said solid material is a cocrystal comprising said cannabinoid and said coformer.

63. The method of claim 62, wherein said cannabinoid is cannabinol, said solid form is crystalline or a cocrystal, and said coformer is selected from the group consisting of potassium, calcium, carnitine, aspartame, L-proline, D-proline, L-Arginine, L-Lysine, betaine, tetramethylpyrazine, 1H-Imidazole, nicotinic acid, saccharin, urea, and nicotinamide.

64-70. (canceled)
